(19) United States
(12) Patent Application Publication
(10) Pub. No.: US 2009/0111766 A1
(43) Pub. Date: Apr. 30, 2009

(54) RAAV VECTOR-BASED COMPOSITIONS AND METHODS FOR THE PREVENTION AND TREATMENT OF MAMMALIAN DISEASES

(75) Inventors: Mark A. Atkinson, Gainesville, FL (US); Terence R. Flotte, Alachua, FL (US); Sihong Song, Gainesville, FL (US); Scott A. Loiler, Gainesville, FL (US)

Correspondence Address: HAYNES AND BOONE, LLP;IP Section, 2323 Victory Avenue, Suite 700, Dallas, TX, 75219, US

(73) Assignee: UNIVERSITY OF FLORIDA RESEARCH FOUNDATION INC., Gainesville, FL (US)

(21) Appl. No.: 12/139,840

(22) Filed: Jun. 16, 2008

Related U.S. Application Data
(63) Continuation of application No. 10/512,008, filed on Aug. 11, 2005, now abandoned.
(60) Provisional application No. 60/374,083, filed on Apr. 19, 2002.

(51) Int. Cl. A61K 31/7088; C12N 15/00

(52) U.S. Cl. 514/44; 435/320.1

(57) ABSTRACT

Disclosed are recombinant adeno-associated viral (rAAV) vector compositions that are expressed in selected mammalian cells, such as pancreatic islets cells, and that encode one or more mammalian serpin or cytokine polypeptides having therapeutic efficacy in the amelioration, treatment and/or prevention of interleukin deficiencies, such as for example diabetes, and related diseases of the pancreas. Also disclosed are methods and compositions for preventing diabetes in a mammal, reducing the rate of disease progression, and ameliorating the symptoms of diabetes in humans at risk for developing such conditions.

The present application claims priority from provisional application Ser. No. 60/374,083 filed Apr. 19, 2002, the entire contents of which is specifically incorporated herein by reference in its entirety.

The United States government has certain rights in the present invention pursuant to grant numbers DK58327, HL59412, RR00082 and DK62652, all from the National Institutes of Health.

### 1. BACKGROUND OF THE INVENTION

1.1 Field of the Invention

The present invention relates generally to the fields of molecular biology and virology, and in particular, to methods for using recombinant adeno-associated virus (rAAV) compositions that express nucleic acid segments encoding therapeutic gene products in the treatment of complex human disorders. In certain embodiments, the invention concerns the use of rAAV in a variety of investigative, diagnostic and therapeutic regimens, including the treatment of diseases of the pancreas and diabetes. Methods and compositions are also provided for preparing rAAV-based vector constructs that comprise one or more therapeutic serpin- or cytokine-encoding gene(s), for use in a variety of viral-based gene therapies, and in particular, treatment and/or prevention of human diseases and disorders such as diabetes.

1.2 Description of Related Art

### 1.2.1 Islet Cells

Type I diabetes is an autoimmune disease characterized by destruction of insulin-producing β cells in the pancreas. Recent findings suggest that autoimmune diseases, including Type 1 diabetes, result not only from defects in recognition of self-antigens but dysregulation of self-reactive immune cells. The pancreatic islets of Langerhans are critical for glucose homeostasis and their loss in Type I diabetes mellitus results in a disease that greatly increases the morbidity and mortality of affected individuals (Atkinson and Eisenbarth, 2001). Islet cell transplantation has provided an approach to the long-term remediation of the condition (Kenyon et al., 1998; Carroll et al., 1995; Ranuncoli et al., 2000). However, the current paradigm of cadaveric donor-derived islet cell transplantation creates a scenario in which allograft immunity compounds pre-existing auto-immunity leading to islet cell destruction.

While certain newer immunosuppressive protocols appear to be better tolerated (Shapiro et al., 2000), it would be highly desirable to enhance islet cell engraftment while decreasing immunosuppressive therapy. This could potentially be accomplished by genetically manipulating the islets to express anti-inflammatory cytokines or other mediators that could act locally to decrease the immune response to the allograft and enhance cell viability (Tahara et al., 1992). Alternatively, insulin gene transfer into hepatocytes in vivo could provide an alternative source of glucose-sensitive insulin release in insulin-deficient type I diabetes.

### 1.2.2 Serpins

Serpin is an acronymic name given to a family of Serine Protease Inhibitors that share a complex, but well conserved, tertiary structure. Members of the serpin family are diversely present in eukaryotes, plants and viruses, and are evident in everyday life from the white of an egg—the non-inhibitory serpin ovalbumin, to the foam protein in beer—the barley Z protease inhibitor. Notably, the serpins are the principal protease inhibitors in human plasma: antithrombin controls the proteolytic coagulation cascade; C1-inhibitor controls complement activation; the plasminogen activator inhibitors, PAI-1 and PAI-2, control fibrinolysis; and α-1-antitrypsin, also called α-1-proteinase inhibitor, modulates connective tissue restructuring.

Altogether the inhibitory serpins make up some 10% in molar terms, of the proteins in human plasma. Also present, in plasma, though in smaller concentrations, are other serpins that have lost their inhibitory activity but have taken on other functions vital to life; examples are the vasopressor peptide source angiotensinogen, and the thyroxine and corticosteroid binding globulins, TBG and CBG.

The reason for the evolutionary success of the serpins is their possession, uniquely amongst the many families of serine protease inhibitors, of a mobile reactive site loop. It is the ability of this loop to profoundly change its conformation that enables the serpins to bind to their target proteases as a virtually irreversible complex.

1.3 Deficiencies in the Prior Art

Currently, there are limited gene-therapy approaches to treating diseases of the pancreas and diabetes in an affected animal using adeno-associated viral delivery vectors. Many such methods introduce undesirable side-effects, and do not overcome the problems associated with traditional modalities and treatment regimens for such conditions. Thus, the need exists for an effective treatment that circumvents the adverse effects and provides more desirable results, with longer acting effects, and improved patient compliance. In addition, methods for delivery of polynucleotides to a host cell that express a gene encoding a therapeutic polypeptide are desirable that are useful in the amelioration of such conditions, and in particular, administration of specific rAAV-based polynucleotide constructs encoding therapeutic cytokines for the treatment and prevention of certain types of diabetes, is particularly desirable.

### 2. SUMMARY OF THE INVENTION

The present invention overcomes these and other limitations inherent in the prior art by providing new rAAV-based genetic constructs specifically suited for transforming mammalian cells, such as human pancreatic islet cells that encode therapeutic and prophylactic polypeptides, and in particular, serpins and/or cytokines that are useful in the treatment and/or prevention of certain types of mammalian diseases and dysfunctions, including, for example, diabetes and other dysfunctions of the pancreas.

In one embodiment, the invention provides an adeno-associated viral vector comprising at least a first polynucleotide that comprises a promoter operably positioned upstream of an isolated nucleic acid segment encoding a biologically-active therapeutic mammalian serpin or cytokine polypeptide, wherein the promoter expresses the nucleic acid segment in a mammalian cell that comprises the vector to produce the encoded mammalian serpin or cytokine polypeptide. The therapeutic polypeptide is preferably selected from the group consisting of elafin, a growth factor, an interferon, an anti-apoptosis factor, and an interleukin. Exemplary therapeutic polypeptides include, but are not limited to, those selected from the group consisting of elafin, BDNF, CNTF, CSF, EGF, FGF, G-SCF, GM-CSF, gonadotropin, IFN, IFG-1, M-CSF, NGF, PDGF, PEDF, TGF, TGF-B2, TNF, VEGF, prolactin, somatotropin, XIAP1. IL-1, IL-2, IL-3, IL-4, IL-5, IL-6, IL-7, IL-8, IL-9, IL-10, viral IL-10, IL-11, IL-12, IL-13, IL-14, IL-15, IL-16, IL-17, and IL-18.

The adeno-associated viral vectors typically will comprise a promoter that is a heterologous, tissue-specific, constitutive or inducible promoter, and in certain embodiments, the preferred promoters include promoters that can express in a variety of human tissues. Exemplary such promoters, for example, in the treatment of diabetes, would include pancreatic-expressible, or an islet-cell-specific promoters. Exemplary heterologous promoters include, but are not limited to, those selected from the group consisting of a CMV promoter, a β-actin promoter, an insulin promoter, a hybrid CMV promoter, a hybrid β-actin promoter, an EF1 promoter, a U1a promoter, a U1b promoter, a Tet-inducible promoter and a VP16-LexA promoter.

The genetic constructs of the invention may also further optionally comprise one or more enhancer sequences operably linked to the nucleic acid segment to enhance expression of the encoded therapeuticum in certain cell types. Exemplary enhancer sequences, include, but are not limited to, a CMV enhancer, a synthetic enhancer, a liver-specific enhancer, a lung-specific enhancer, a muscle-specific enhancer, a kidney-specific enhancer, a pancreas-specific enhancer, or an islet cell-specific enhancer.

The rAAV vectors may also further optionally comprise one or more post-transcriptional regulatory sequences, such as the woodchuck hepatitis virus post-transcription regulatory element.

The invention also provides recombinant adeno-associated virus virions an pluralities of rAAV viral particles that comprise at least a first therapeutic AAV construct as disclosed herein. The rAAV particles may be of any of the known serotypes, such as for example, AAV serotype 1, AAV serotype 2, AAV serotype 3, AAV serotype 4, AAV serotype 5, and AAV serotype 6, while virions of the 2^{nd} serotype, AAV2 are particularly contemplated to be useful in the practice of the invention.

A further aspect of the invention concerns mammalian cells that comprise at least one of the rAAV vectors, virions, or viral particles disclosed herein. Although all mammalian cells are contemplated to be useful in the present invention, in certain embodiments, exemplary mammalian cells include, endothelial cells, islet cells, hepatocytes, pancreatic cells, renal cells, myocytes, splenic cells, biliary cells, cardiac cells, pulmonary cells, and neural cells. Preferably such cells are human cells.

As described hereinbelow, the invention also provides compositions and kits that comprise one or more of the disclosed vectors, virions, viral particles, or host cells of the invention. Typically such compositions will further comprise at least a first pharmaceutical excipient, buffer, or diluent, and may be formulated for administration to a human, or an animal under veterinary care. Such compositions may further optionally comprise one or more additional therapeutic compounds, compositions, or medicaments, and may be formulated for use in the prophylaxis or therapy of a variety of diseases, disorders, or dysfunctions, such as, for example, for use in cancer, diabetes, autoimmune disease, pancreatic disease, or liver disease therapy.

The compositions as disclosed herein may further comprise at least a first liposome, lipid, lipid complex, microsphere, microparticle, nanosphere, or nanoparticle, as may be desirable to facilitate or improve delivery of the therapeuticum to one or more cell types, tissues, or organs in the animal to be treated.

In addition to the vectors, compositions, host cells, and kits described above, the invention also pertains to the use of such compositions in the treatment and/or prophylaxis of a number of diseases. In a general sense, the methods of the invention concern means for preventing, treating or ameliorating the symptoms of a disease, dysfunction, or deficiency in a mammal. The methods generally involve providing to or administering to the mammal a composition that comprises the virions or the viral particles as disclosed herein in an amount and for a time sufficient to treat or ameliorate the symptoms of the disease, dysfunction, or deficiency in the mammal. In illustrative embodiments, the mammal has, is diagnosed with, or is at risk for developing, diabetes, an autoimmune disorder, a cytokine deficiency, a serpin deficiency, or an interleukin deficiency.

Although all mammals may find benefit of the present invention, in preferred embodiments, the animal is a human being that has, has been diagnosed with, or is at risk for developing one or more such disorders.

In the methods of the invention, the virions or plurality of viral particles, or one or more compositions comprising them are provided to, or administered to, the mammal by a suitable delivery means. Exemplary means for delivering rAAV particles to a mammal, include, for example, by intramuscular, intravenous, subcutaneous, intrathecal, intraperitoneal, or intracerebroventricular administration, or by direct injection into one or more tissues or organs, such as for example, by injection into the tissues or cells of the pancreas, the liver, the heart, the lungs, the brain or spinal cord, one or both kidneys, into the bones or joints, or, into the muscles or subcutaneous spaces.

The invention also provides a method for treating diabetes in a mammal suspected of having, or at risk for developing diabetes. The method generally involves providing to such a mammal one or more of the therapeutic rAAV compositions disclosed herein, in an amount and for a time sufficient to treat or ameliorate the symptoms of the diabetes in the mammal. Preferred animals include those under veterinary care, as well as human beings under the care of a physician, and particularly those with a familial history of diabetes, or those at risk for developing it.

The invention also provides a method for preventing Type I diabetes in a human suspected of having, or at risk for developing Type I diabetes. The method generally involves prophylactically administering to such a patient one or more of the therapeutic raAAV compositions disclosed herein, in an amount and for a time sufficient to prevent, delay the onset of, reduce the seriousness of, or lessen the severity of Type I diabetes in the patient. Similarly, the invention provides rAAV vectors and compositions for use in methods for reducing the rate of disease progression of Type I diabetes in a human diagnosed with Type I diabetes. Such methods generally involve at least the step of providing to or administering to the patient, an effective amount of one of the disclosed therapeutic AAV compositions for a time sufficient to reduce the rate of disease progression of Type I diabetes in the human. Such administration may involve a single administration, or as needed, may be divided over multiple administrations to achieve the desired therapeutic effects.

In illustrative embodiments, the improved rAAV constructs of the invention comprise at least a first polynucleotide that comprises a promoter and/or enhancer region positioned upstream of, and operably linked to, a nucleic acid segment that encodes one or more biologically-active mammalian serpin or cytokine polypeptides. These vectors also preferably comprise a β-actin promoter sequence operably linked to a gene encoding a mammalian serpin or cytokine polypeptide.

The invention also provides methods for treating or ameliorating such a polypeptide deficiency in a mammal, and particularly for preventing, treating or reducing the severity or extent of deficiency in a human manifesting one or more of the disorders linked to a deficiency in such polypeptides in cells and tissues of a human. In a general sense, the method involves administration of an rAAV-based genetic construct that specifically encodes one or more therapeutic serpin or cytokine polypeptides in a pharmaceutically-acceptable vehicle to the animal in an amount and for a period of time sufficient to prevent, treat or ameliorate the symptoms of certain types of diabetes in the animal suspected of suffering from such a disorder. In particular the invention contemplates the treatment and/or prevention of diabetes and related disorders by specifically providing to pancreatic islet cells prophylactically- and therapeutically-effective amounts of rAAV vectors that comprise polynucleotide segments that express therapeutically-effective amounts of mammalian serpins or cytokines, including, for example, AAT1, elafin, and IL-4 and IL-10.

In other embodiments, a polynucleotide encoding one or more therapeutic cytokine polypeptides, such as BDNF, CNTF, CSF, EGF, FGF, G-SCF, GM-CSF, gonadotropin, IFN-α, IFN-β, IFN-γg, IGF-I, IGF-II, M-CSF, NGF, PDGF, PEDF, TGF, TGF-β2, TNF, VEGF, prolactin, somatotropin, or XIAP1 is placed under the control of the suitabler promoter and used to produce therapeutically-effective levels of the biologically-active encoded therapeutic polypeptide when suitable mammalian cells comprise the rAAV genetic construct.

In other embodiments, a polynucleotide encoding one or more therapeutic serpin polypeptides, such as any one of those described in Section 5.5 hereinbelow, and as included in SEQ ID NOs:1-SEQ ID NO:50 is placed under the control of the suitabler promoter, and used to produce therapeutically-effective levels of the biologically-active encoded therapeutic polypeptide in suitable mammalian cell that comprise the rAAV genetic construct. Such constructs are useful not only in therapy, but may also be important in the prophylaxis or prevention of certain types of diseases in affected mammals.

The vector constructs of the present invention may also further optionally comprise one or more native, synthetic, or hybrid enhancer elements, for example, a CMV enhancer, a synthetic enhancer, or a tissue- or cell-specific enhancer, such as for example, a pancreatic cell, or an islet-cell-specific promoter, such as the human insulin promoter.

The vector constructs of the present invention may also further optionally comprise one or more native, synthetic, or hybrid post-transcriptional regulatory elements that may function to help stabilize the RNA and increase overall expression of the therapeutic polypeptide. An exemplary such element is the woodchuck hepatitis virus post-transcriptional regulatory element (WPRE) (see Paterna et al., 2000 and Loeb et al., 1999).

The vectors may also further optionally comprise one or more intron sequences to facilitate improved expression of the therapeutic genes placed under the control of the promoter and/or promoter/enhancer regulatory regions.

In illustrative embodiments, the invention concerns administration of one or more biologically active cytokine polypeptides that comprise an at least 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500 or more contiguous amino acid sequence from any one of the amino acid sequences encoding a biologically-active mammalian serpin or cytokine polypeptide as described herein.

Other aspects of the invention concern recombinant adeno-associated virus virions, viral particles, host cells, and compositions that comprise one or more of the vectors, virions, or viral particles disclosed herein, such as for example pharmaceutical formulations of the vectors intended for administration to a mammal through suitable means, such as, by intramuscular, intravenous, or direct injection to one or both cells, tissues, organs, or organ systems of a selected mammal. Typically, such compositions will be formulated with pharmaceutically-acceptable excipients as described hereinbelow, and may comprise one or more liposomes, lipids, lipid complexes, microspheres or nanoparticle formulations to facilitate administration to the selected organs, tissues, and cells for which therapy is desired.

Therapeutic and prophylactic kits for preventing, treating or ameliorating the symptoms of a mammalian disease, disorder, or dysfunction, such as for example, a cytokine, serpin, or n interleukin deficiency also form important aspects of the present invention. Such kits typically comprise one or more of the disclosed AAV vector constructs, virions, virus particles, host cells, or compositions described herein, and instructions for using the kit.

Another important aspect of the present invention concerns methods of use of the disclosed vectors, virions, compositions, and host cells described herein in the preparation of medicaments for treating or ameliorating the symptoms of such a disease or dysfunction, or other conditions resulting from an interleukin polypeptide deficiency condition in a mammal. Such methods generally involve administration to a mammal, or human in need thereof, one or more of the disclosed vectors, virions, host cells, or compositions, in an amount and for a time sufficient to treat or ameliorate the symptoms of such a deficiency in the affected mammal. The methods may also encompass prophylactic treatment of animals suspected of having such conditions, or administration of such compositions to those animals at risk for developing such conditions either following diagnosis, or prior to the onset of symptoms. Such symptoms may include, but are not limited to, diabetes, rheumatoid arthritis, lupus, hyperinsulinemia, hypoinsulinemia, liver dysfunction, and a variety of autoimmune disorders.

### 2.1 Therapeutic Polypeptides and Compositions Thereof

The present invention provides rAAV vectors that encode one or more therapeutic polypeptides that comprise, consist essentially of, or consist of, at least a first sequence region that preferably shares at least about 70%, about 71%, about 72%, about 73%, about 74%, about 75%, about 76%, about 77%, about 78%, about 79%, or about 80% or higher sequence identity with the amino acid sequence of any one of SEQ ID NO:1 to SEQ ID NO:50. Likewise, the present invention provides rAAV vectors that encode one or more therapeutic polypeptide that comprise, consist essentially of, or consist of, at least a first sequence region that preferably shares at least about 81%, about 82%, about 83%, about 84%, about 85%, about 86%, about 87%, about 88%, or about 90%, or higher sequence identity with the amino acid sequence of any one of SEQ ID NO:1 to SEQ ID NO:50. The invention also provides rAAV vectors that encode one or more therapeutic polypeptides that comprise, consist essentially of, or consist of, at least a first sequence region that preferably shares at least about 91%, about 92%, about 93%, about 94%, about 95%, about 96%, about 97%, about 98%, or about 99%, or higher sequence identity with the amino acid sequence of any one of SEQ ID NO:1 to SEQ ID NO:50.

Such polypeptides may be used in a variety of embodiments, methods, and uses, and particularly in those methods and uses as described herein. Highly preferred polypeptides and proteins of the invention include those peptides and polypeptides that comprise all, substantially all, or an antigenic fragment from, at least a first human therapeutic polypeptide. Highly preferred polypeptides are those that comprise, consist essentially of, or consist of, at least a first sequence region that comprises an at least about 27, an at least about 28, an at least about 29, an at least about 30, an at least about 31, or an at least about 32 or more contiguous amino acid sequence from any one of SEQ ID NO:1 to SEQ ID NO:50, and particularly those biologically-active polypeptides that provide therapeutic or prophylactic benefit when expressed in a suitable mammalian host cells under the appropriate conditions for such activity.

Likewise, rAAV vectors that encode one or more therapeutic polypeptides that comprise, consist essentially of, or consist of, at least a first sequence region that comprises an at least about 33, 34, 35, 36, 37, 38, 39, or 40 or more contiguous amino acid sequence from any one of SEQ ID NO:1 to SEQ ID NO:50, are also highly preferred in the practice of the present invention, as are those that comprise, consist essentially of, or consist of, at least a first sequence region that comprises an at least about 41, 42, 43, 44, 45, 46, 47, 48, 49, or 50 or more contiguous amino acid sequence from any one of SEQ ID NO:1 to SEQ ID NO:50, so long as the therapeutic or prophylactic biological activity of the protein or polypeptide is maintained.

As such, exemplary highly-preferred rAAV vectors are those that encode polypeptides that comprise, consist essentially of, or consist of, at least a first sequence region that comprises an at least about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more contiguous amino acid sequence from any one of SEQ ID NO:1 to SEQ ID NO:50 (and even up to and including the full-length or substantially-full length sequences of any one of SEQ ID NO:1 to SEQ ID NO:50, and that possess therapeutic or prophylactic biological activity when expressed in a suitable mammalian host cell under the appropriate conditions for such enzymatic activity.

### 2.2 Therapeutic Polypeptide-Encoding Polynucleotide Compositions

Highly preferred polynucleotides are those that comprise at least a first nucleic acid segment that comprises, consists essentially of, or consists of, a sequence that encodes a polypeptide that comprises an at least about 30 contiguous amino acid sequence from any one of SEQ ID NO:1 to SEQ ID NO:50 are contemplated to be particularly preferred in the methods of the present invention.

Naturally, all intermediate contiguous sequences are contemplated to fall within the scope of the present invention. For example, polynucleotides that comprise, consist essentially of, or consist of, a sequence that encodes a polypeptide that comprises at least at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 85, at least about 90, at least about 95, or at least about 100 or more contiguous amino acids from any one of SEQ ID NO:1 to SEQ ID NO:50 are contemplated to be particularly preferred in the methods of the present invention, and are contemplated to be particularly preferred polynucleotide compositions.

The invention provides rAAV vectors that comprise at least a first isolated nucleic acid segments that: encodes a therapeutic or prophylactic polypeptide that comprises an at least 15 contiguous amino acid sequence from any one of SEQ ID NO:1 to SEQ ID NO:50; or a polynucleotide that hybridizes to a sequence that encodes the polypeptide of any one of SEQ ID NO:1 to SEQ ID NO:50, or that hybridizes to the complement thereof, under stringent hybridization conditions.

The isolated polynucleotides of the invention preferably comprise at least a first sequence region that encodes at least a first peptide or polypeptide that has at least about 70%, 71%, 72%, 73%, 74%, 75%, 76%, 77%, 78%, 79% or 80% or greater sequence identity with the amino acid sequence of any one of SEQ ID NO:1 to SEQ ID NO:50. More preferably, the polynucleotides of the invention preferably comprise at least a first sequence region that encodes at least a first peptide or polypeptide that has at least about 81%, 82%, 83%, 84%, 85%, 86%, 87%, 88%, 89%, or 90% or greater sequence identity with the amino acid sequence of any one of SEQ ID NO:1 to SEQ ID NO:50. More preferably still, the polynucleotides of the invention preferably comprise at least a first sequence region that encodes at least a first peptide or polypeptide that has at least about 91%, 92%, 93%, 94%, 95%, 96%, 97%, 98%, or 99% or greater sequence identity with the amino acid sequence of any one of SEQ ID NO:1 to SEQ ID NO:50.

The disclosed polynucleotides may encode native or synthetically-modified peptides, proteins, antisense molecules, or ribozymes, or may encode one or more biologically-active, or therapeutically-effective variants thereof as described herein.

Such biologically-active variants, and the polynucleotides encoding them preferably contain nucleotide substitutions, deletions, insertions and/or additions that change no more than about 25%, more preferably at no more than about 20% or 15%, and more preferably still, at no more than about 10% or 5%, of the nucleotide positions relative to the corresponding polynucleotide sequence that encodes the native unmodified polypeptide sequence. Certain polynucleotide variants, of course, may be substantially homologous to, or substantially identical to the corresponding region of the nucleotide sequence encoding an unmodified peptide. Such polynucleotide variants are capable of hybridizing to a naturally occurring DNA sequence encoding the selected sequence under moderately stringent, to highly stringent, to very highly stringent conditions.

Suitable moderately stringent conditions include pre-washing in a solution containing about 5×SSC, 0.5% SDS, 1.0 mM. EDTA (pH 8.0); hybridizing at a temperature of from about 50° C. to about 60° C. in 5×SSC overnight; followed by washing twice at about 60 to 65° C. for 20 min. with each of 2×, 0.5× and 0.2×SSC containing 0.1% SDS). Suitable highly stringent conditions include pre-washing in a solution containing about 5×SSC, 0.5% SDS, 1.0 mM EDTA (pH 8.0); hybridizing at a temperature of from about 60° C. to about 70° C. in 5×SSC overnight; followed by washing twice at about 65 to 70° C. for 20 min. with each of 2×, 0.5× and 0.2×SSC containing 0.1% SDS). Representative examples of very highly stringent hybridization conditions may include, for example, pre-washing in a solution containing about 5×SSC, 0.5% SDS, 1.0 mM EDTA (pH 8.0); hybridizing at a temperature of from about 70° C. to about 75° C. in 5×SSC overnight; followed by washing twice at about 70° C. to about 75° C. for 20 min. with each of 2×, 0.5× and 0.2×SSC containing 0.1% SDS). Such hybridizing DNA sequences are also within the scope of this invention.

It will be appreciated by those of ordinary skill in the art that, as a result of the degeneracy of the genetic code, there are many nucleotide sequences that encode a given therapeutic polypeptide. Some of these polynucleotides bear minimal homology to the nucleotide sequence of any native gene. Nonetheless, polynucleotides that vary due to differences in codon usage are specifically contemplated by the present invention.

Polypeptide-encoding polynucleotides may also be synthesized by any method known in the art, including chemical synthesis (e.g., solid phase phosphoramidite chemical synthesis). Modifications in a polynucleotide sequence may also be introduced using standard mutagenesis techniques, such as oligonucleotide-directed site-specific mutagenesis (Adelman et al., 1983). Alternatively, RNA molecules may be generated by in vitro or in vivo transcription of DNA sequences encoding a therapeutic polypeptide, provided that the DNA is incorporated into a vector with a suitable RNA polymerase promoter (such as T7 or SP6). Certain portions may be used to prepare an encoded peptide, as described herein. In addition, or alternatively, a portion may be administered to a patient such that the encoded peptide is generated in vivo (e.g., by transfecting antigen-presenting cells such as dendritic cells with a cDNA construct encoding a therapeutic polypeptide, and administering the transfected cells to the patient).

Polynucleotides that encode a therapeutic polypeptide may generally be used for production of the polypeptide, in vitro or in vivo. Polynucleotides that are complementary to a coding sequence (i.e., antisense polynucleotides) may also be used as a probe or to inhibit the biological activity of a particular selected sequence. cDNA constructs that can be transcribed into antisense RNA may also be introduced into cells of tissues to facilitate the production of antisense RNA.

Any of the disclosed polynucleotides may be further modified to increase stability in vivo. The is particularly relevant when the therapeutic construct delivered by the disclosed AAV vectors is an antisense molecular or a ribozyme. In such cases, possible modifications include, but are not limited to, the addition of flanking sequences at the 5′ and/or 3′-ends; the use of phosphorothioate or 2′-o-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors, and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other poxvirus (e.g., avian poxvirus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

### 2.3 Pharmaceutical Compositions

The genetic constructs of the present invention may be prepared in a variety of compositions, and may also be formulated in appropriate pharmaceutical vehicles for administration to human or animal subjects. The AAV molecules of the present invention and compositions comprising them provide new and useful therapeutics for the treatment, control, and amelioration of symptoms of a variety of disorders. Moreover, pharmaceutical compositions comprising one or more of the nucleic acid compounds disclosed herein, provide significant advantages over existing conventional therapies—namely, (1) their reduced side effects, (2) their increased efficacy for prolonged periods of time, (3) their ability to increase patient compliance due to their ability to provide therapeutic effects following as little as a single administration of the selected therapeutic AAV composition to affected individuals. Exemplary pharmaceutical compostions and methods for their administration are discussed in significant detail hereinbelow.

The invention also provides compositions comprising one or more of the disclosed vectors, expression systems, virions, viral particles; or mammalian cells. As described hereinbelow, such compositions may further comprise a pharmaceutical excipient, buffer, or diluent, and may be formulated for administration to an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a mammal in need thereof. Such compositions may be formulated for use in therapy, such as for example, in the amelioration, prevention, or treatment of conditions such as peptide deficiency, polypeptide deficiency, cancer, diabetes, autoimmune disease, pancreatic disease, or liver disease or dysfunction.

Use of one or more of the disclosed compositions in the manufacture of medicaments for treating a variety of diseases is also an important aspect of the invention. Such diseases include, for example, cancer, diabetes, cardiovascular diseases including coronary heart disease, angina, myocardial infarction, ischemias, restenosis, and strokes, atherosclerosis, pulmonary and circulatory diseases, including cystic fibrosis, hyperinsulinemia, hypoinsulinemia, adiposity, autoimmune diseases, lupus, inflammatory bowel disease, pancreatic dysfunction, hepatic dysfunction, biliary dysfunction and diseases, as well as neurological diseases including for example, Parkinson's, Alzheimer's, memory loss, and the like, as well as musculoskeletal diseases including, for example, arthritis, ALS, MLS, MD, and such like, to name only a few.

### 3. BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1 shows endogenous expression of α-1 antitrypsin (AAT) in human islet cells. Human islet cells cultured in 24-well plate (100 islets/well, n=3) with 1 ml of RPMI medium containing 10% FBS. Human AAT in the medium was measured by ELISA every 2 days.

FIG. 2A and FIG. 2B show AAV2-CMV-IL-4 and IL-10 constructs and expression from these constructs after transfection into intact human islet cells. FIG. 2A shows vector cassette map where ITR=AAV inverted terminal repeat, CMVp=CMV immediate early promoter. The box following the promoter is the CMV 1^{st} intron, and the box following the gene is the SV40 polyA signal. FIG. 2B shows the concentrations of IL-4 and IL-10 48 hr after transduction of 0.2×10³ islets in a 35-mm well measured by antigen capture ELISA are shown (mean of three experiments, performed in duplicate).

FIG. 3 shows the effect of rAAV transduction on glucose stimulated insulin release. Insulin concentrations in culture medium of islets transduced with the rAAV vectors and/or adenovirus.

FIG. 4 shows transductions of AAV 1 to 5 in murine islet cells. Isolated islet cells from C57bl/6j mice were transduced with AAV vectors expressing hAAT (1×10⁹ particles/80 islets) and cultured in 24-well plate (80 islets/well, n=3) with 1 ml of RPMI medium containing 10% FBS. Human AAT in the medium was measured by ELISA 6 days after transduction.

FIG. 5 shows long-term transgene expression in murine skeletal muscle transduced with rAAV. Eight-week-old male C57BL/6 and C57BL/6-SCID mice were injected with 1.4×10¹³ particles of rAAV-hAAT vector (C-AT). Serum levels of hAAT were measured by ELISA.

FIG. 6 shows IL-10 shows Type I diabetes in NOD mice undergoing various treatment modalities. These life table analyses demonstrate the percentage of mice (n=10 for each group) remaining normoglycemic after injection with saline (dashed/open triangle line); rAAV-IL-4 (dashed/closed square); rAAV-IL-10 (solid/closed triangle); or the combination of rAAV-IL-4 and rAAV-IL-10 (solid/open square). *, P<0.005 vs. the control group and the rAAV-IL-4 treated group.

FIG. 7A and FIG. 7B shows rAAV cytokine gene delivery and the natural history of insulin autoantibodies (IAA) in NOD mice. Longitudinal analysis of animals followed from 4 wk until 16 wk or later: saline (FIG. 7A); rAAV-IL-10 (FIG. 8B) (developed diabetes, closed circle; no diabetes, open circle). The dashed line represents the definition for positive IAA responses. P<0.03 for IL-10 vs. saline controls based on the frequency of IAA positive animals at 12 or 16 wk.

FIG. 8 shows hAAT gene transfer prevents Type I diabetes in NOD mice. These life table analyses demonstrate the percentage of mice (n=10 for each group) remaining normoglycemic after injection with rAAV2-CB-AT vector (1×10¹⁰ i.u./mouse) or saline.

FIG. 9 shows hAAT gene transfer reduces insulitis. Histogram depicts percentage of normal islets (stage 1, unfilled bar), peri-insulitis (stage 2, light gray bar), insulitis involving <50% of the islet in cross section (stage 3, dark gray bar), or insulitis involving >50% of the islet (stage 4, black bar).

FIG. 10 shows the natural history of insulin auto-antibodies in NOD mice transduced with rAAV2-CB-AT. Longitudinal analysis of animals followed from 4 wk until 16 wk or later. Developed diabetes, closed circle; no diabetes, open circle. The dashed line represents the definition for positive IAA responses.

FIG. 11 shows comparison of muscle cell transductions with five serotypes of rAAV-CB-AT vector.

FIG. 12 shows recombinant AAV vector cassettes. An, polyA signal; ITR, AAV2-inverted terminal repeat sequences; CMV-β-actin promoter, CMV enhancer and chicken β-actin promoter with a hybrid chicken β-globin intron.

FIG. 13 shows strategies for vector administration and islet transplantation.

FIG. 14A, FIG. 14B, FIG. 14C and FIG. 14D show rAAV-CMV-IL-4, rAAV-CMV-IL-10, and rAAV-CB-IL-10 constructs and expression in mouse myoblasts. FIG. 14A shows vector cassette map where ITR, rAAV inverted terminal repeat, CMVp, CMV immediate early promoter, and CBp, CMV enhancer and chicken β-actin promoter with a hybrid chicken-rabbit β-globin intron. The circle after the gene is the simian virus 40 poly(A) signal. FIG. 14A also shows several illustrative embodiments that utilize a mutated form of IL-10, an isoleucine to alanine mutation at amino acid 87 [IL-10 (I87A)]. FIG. 14B shows the concentrations of IL-4 and IL-10 48 hr after plasmid (pCMV-green fluorescent protein, pCMV-IL-4, pCMV-IL-10) transfection of C2C12 cells (performed in triplicate). FIG. 14C and FIG. 14D show the concentrations of IL-4 (FIG. 14C) and IL-10 (FIG. 14D) 0-3 days after viral (rAAV-CMV-IL-4, rAAV-CB-IL-10) transduction of C2C12 cells (performed in triplicate). Transductions with rAAV alone (multiplicity of infection 2,000) or under coinfection with rAAV (multiplicity of infection 2,000) and Ad5 (multiplicity of infection 5).

FIG. 15A, FIG. 15B, FIG. 15C, FIG. 15D, FIG. 15E and FIG. 15F show rAAV cytokine gene delivery and the natural history of insulin autoantibodies in NOD mice. Longitudinal analysis of animals followed from 4 until 16 weeks or later. Saline (FIG. 15A); rAAV-IL-10 (FIG. 15B); rAAV-IL-4 (FIG. 15C) (developed diabetes, ; no diabetes, ◯). The dashed line represents the definition for positive IAA responses. Life-table presentation of animals as a function of treatment group: saline (FIG. 15D); rAAV-IL-10 (FIG. 15E); rAAV-IL-4 (FIG. 15F) (ever IAA positive, ; never IAA positive, ◯). P<0.03 for IL-10 vs. saline controls based on the frequency of IAA-positive animals at 12 or 16 weeks.

FIG. 16A, FIG. 16B, FIG. 16C, FIG. 16D, FIG. 16E, FIG. 16F, FIG. 16G, FIG. 16H, FIG. 16I, FIG. 16J, FIG. 16K, FIG. 16L and FIG. 16M show the effect of rAAV cytokine gene delivery in skeletal muscle on splenocyte function. Splenocyte responses in the absence of Con A (U; untreated) or at two different Con A concentrations (1 and 10 μg/ml) at 24 (clear bar) and 48 hr (solid bar) after stimulation are shown. FIG. 16A, FIG. 16B and FIG. 16C show IL-2 production in saline-treated (FIG. 16A), rAAV-IL-4-treated (FIG. 16B), and rAAV-IL-10-treated (FIG. 16C) mice. FIG. 16D, FIG. 16E and FIG. 16F show IL-4 production in saline-treated (FIG. 16D), rAAV-IL-4-treated (FIG. 16E), and rAAV-IL-10-treated (FIG. 16F) mice. FIG. 16G, FIG. 16H and FIG. 16I show IL-10 production in saline-treated (FIG. 16G), rAAV-IL-4-treated (FIG. 16H), and rAAV-IL-10-treated (FIG. 16I) mice. FIG. 16J, FIG. 16K and FIG. 16L show IFN-γ production in saline-treated (FIG. 16J), rAAV-IL-4-treated (FIG. 16K), and rAAV-IL-10-treated (FIG. 16L) mice. *, P=0.01 vs. control group. Note that statistical comparisons were made with the use of “peak” concentrations (1 or 10 μg/ml) at 24 and 48 hr only. FIG. 16M shows life-table analysis of incidence of hyperglycemia in irradiated male NOD mice adoptively transferred with splenocytes from NOD mice recently diagnosed with Type I diabetes () or 30-week-old rAAV-IL-10-treated NOD mice.

FIG. 17 shows introduction of recombinant transgenes via rAAV demonstrate sustained expression in vivo. FIG. 17 shows the mean total serum levels of AAT observed in groups of either SCID (squares) or C57Bl/6 (circles) mice receiving either low dose (open symbols) (5×10¹¹ DNase resistant particles per mouse) or high dose (1.4×10¹³ DNase resistant particles per mouse) (filled symbols) single injections of the CMV-AT vector measured at time points in weeks post-injection. For each strain, the high-dose curve is significantly different from the low-dose curve (P=0.009 for SCID, P=0.02 for C57Bl/6), but the strains do not differ significantly from each other.

FIG. 18 shows NOD into diabetic NOD female islet transplants. Islets were obtained from young (6-8 weeks) NOD males. Islets were implanted under the kidney capsule of recipients (approximately 700 islet equivalents). Treatment with anti-CD154 was started the day before transplant and continued indefinitely (or until diabetes occurrence) at weekly intervals. Graft survival was significantly improved in anti-CD154 treated group versus controls (p=0.016 vs. Ha 4/8; p=0.0007 vs. saline).

FIG. 19A, FIG. 19B and FIG. 19C are dose and time study when female NOD mice were injected with CB-IL-10 (indicated doses) at 12 weeks of age (right before onset of diabetes) (FIG. 19A) at 8 weeks of age (FIG. 19B) and at 4 weeks of age (FIG. 19C).

### 4. DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### 4.1 Type I Diabetes

Type I diabetes is an autoimmune disease characterized by destruction of insulin-producing β cells in the pancreas. Recent findings suggest that autoimmune diseases, including Type I diabetes, result not only from defects in recognition of self-antigens but dysregulation of self-reactive immune cells. In fact, using rAAV vector mediated gene therapy, others have confirmed that the anti-inflammatory cytokine IL-10 prevents the development of Type I diabetes in NOD mice (Goudy et al., 2001), as well as recurrence of Type I diabetes after syngeneic islet transplantation in NOD mice.

### 4.2 NOD Mice as a Model for Type I Diabetes

The NOD mouse provides a well-accepted model system to investigate disease pathology and intervention strategies to prevent human Type I diabetes (Atkinson and Maclaren, 1994). Beginning at approximately five weeks of age, a mononuclear cell infiltrate of the pancreatic ducts and venules initiates with eventual progression to the pancreatic islets (i.e., insulitis). Whereas these early insulitis stages appear “non-destructive,” intra-islet invasion occurs at 12-16 weeks of age with this latter infiltrate associated with selective destruction of the insulin-secreting β cells. The cellular infiltrate is heterogeneous, with a predominance of T cells followed by various percentages of macrophages, dendritic cells and B-lymphocytes. Multiple lines of evidence suggest that both CD4+T-helper and CD8+T-cytotoxic lymphocytes play a role in the disorder (Bendelac et al., 1987; Miller et al., 1988; Wang et al., 1987; Like et al., 1986; Sibley and Sutherland, 1987; Haskins et al., 1988). Evidence for spontaneous β cell regeneration is limited, and allogenic islets transplanted into diabetic recipients undergo a repeated episode of islet cell destruction.

### 4.3 AAT and its Anti-Inflammatory Property

Alpha-1 antitrypsin (AAT) is the first and main member of the serpin (serine proteinase inhibitor) superfamily, in which there are over 60 members occurring widely in higher organisms, viruses, insects and plants. Overall, the serpins have strong conservation of their internal residues and their tertiary structure. The irreversibility of proteinase inhibition achieved by the serpins has made them the principal inhibitors controlling both intra- and extracellular proteolytic pathways. Serpins regulate such diverse physiological processes as coagulation, fibrinolysis, inflammation and neoplasia. Serpin dysfunction has been implicated in thrombosis, emphysema, cirrhosis, immunohypersensitivity, mental disorders and in diseases characterized by connective and other tissue self-destruction (Stein and Carrell, 1995; Janciauskiene, 2001). Some serpins, such as ovine uterine serpin, inhibit a wide variety of immune responses, including mixed lymphocyte reaction, mitogen-stimulated lymphocyte proliferation, T cell-dependent antibody production and immunological rejection of the fetal allograft (Peltier and Hansen, 2001). It is becoming clear that serpins have immunosuppressive activity in addition to their role as proteinase inhibitors (Janciauskiene, 2001).

AAT is a 52-kDa glycoprotein. AAT can inhibit neutrophil elastase and proteinase 3 with high efficiency, and cathepsin G, thrombin, trypsin and chymotrypsin with lower efficiency (Macen et al., 1993). It is primarily synthesized in the liver, but can also be produced by extrahepatic cells including neutrophils, monocytes, macrophages, alveolar macrophages, intestinal epithelial cells, carcinoma cells and the cornea (Ray et al., 1977; Geboes et al., 1982; Keppler et al., 1996; Boskovic and Twining, 1998). The normal serum level of AAT in humans is 2-3 mg/ml. During inflammation, infection and malignant diseases, AAT levels, as an acute phase reactant, can rise by 3- to 4-fold. It has been shown that in human neutrophils, monocytes, and alveolar macrophages, AAT expression increases in response to inflammatory mediators such as IL-6, lipopolysaccharide and itself when complexed with neutrophil elastase (Perlmutter and Punsal, 1988; Knoell et al., 1998). Under the inflammatory conditions, AAT also can be chemically modified by nitric oxide (NO) and exhibit antibacterial and cystein protease inhibitor activities (Miyamoto et al., 2000). It has been observed that AAT completely abolished the acute inflammatory infiltration and connective tissue breakdown (Dhami et al., 2000). Churg et al. (2001) recently demonstrated that human AAT completely suppressed silica-induced PMN influx into the lung and macrophage inflammatory protein-2 (MIP-2)/monocyte chemotactic protein-1 (MCP-1) gene expression and partially suppressed nuclear transcription factor κB (NF-κB) translocation and increased inhibitor of NF-κB (I-κB) levels in mouse model. It has also been demonstrated that adenovirus mediated AAT gene transfer significantly decreases neointima formation after mechanical dilation, and reversed the local inflammation that characterized viral controls (Waugh et al., 2001). Increasing evidence indicates that anti-inflammatory properties of AAT may render it as a therapeutic drug for altering immune system and protecting tissue transplantation.

### 4.4 Elafin and its Anti-Inflammatory Property

Elafin (neutrophil elastase inhibitor) was originally isolated from the scales of patients with psoriasis (Wiedow et al., 1990) and in lung secretions (Sallenave and Ryle, 1991; Tremblay et al., 1996), but it is also present at mucosal sites in many tissues. It presents in sputum, in tracheal biopsies and bronchoalveolar lavage from both normal subjects and patients, and its synthesis by Clara cells and type II cells in lung. It has recently been observed that macrophages also express elafin. Elafin is a 6-kDa peptide. The sequence of the gene showed that it is approximately 2.3-kb long, and is composed of three exons and two introns. The 5′ regulatory sequences contain activator protein-1 and nuclear factor-B sites. A positive regulatory cis-element present in the region between −505 and −368 bp is responsible for the upregulation of the elafin gene in normal breast epithelial cells. The peptide is composed of 117 amino acid residues including a hydrophobic signal peptide of 22 residues. Elafin can be divided into two domains, the carboxy-terminal domain containing the antiproteinase active site and the amino-terminal domain containing characteristic VKGQ sequences. These sequences allow the elafin molecule to glue itself into polymers and bind other interstitial molecules through transglutamination. This feature could make elafin maximally effective as a tissue-bound inhibitor as opposed to AAT, which is present in large amounts in the circulation. Elafin has also been suggested to have a locally protective role against neutrophilic damage, presumably because of its small size and negative charge. Elafin has been shown to be more specific in its spectrum. It inhibits pancreatic elastase, neutrophil elastase and proteinase-3.

In addition to its proteinase inhibitory properties and given its biochemical characteristics, elafin also has immunoregulatory properties. In vivo, elafin is active against Pseudomonas aeruginosa. Over expression of elafin in transgenic mice reduces myocardial inflammatory damage and mortality following viral myocarditis (Zaidi et al., 1999). Administration of elafin to rabbits after heart transplantation limits neointimal formation in coronary arteries by preventing both the migration and proliferation of vascular smooth muscle cells (Cowan et al., 1996). It has also been shown that inflammatory cell infiltration is associated with serine elastase activity in rabbit vein grafts. Gene transfer of elafin in vein grafts is effective in reducing the early inflammatory response and against atherosclerotic degeneration (O'Blenes et al., 2000). This protective effect may be employed in islet transplantation.

4.5 AAV Biology and Recombinant AAV (rAAV) Vectors for Gene Transfer

Adeno-associated virus (AAV) is a single-stranded DNA parvovirus with a 4.7 kb genome and a particle diameter of approximately 20 nm. The AAV genome is flanked by two identical inverted terminal repeat (ITR) sequences (Lusby et al., 1980). These ITRs provide all the cis-acting sequence required for replication, packaging and integration (Samulski et al., 1989). There are two large open reading frames (Srivastava et al., 1983). The open reading frame in the right half of the genome (cap) encodes 3 overlapping coat proteins (VP1, VP2 and VP3). The open reading frame in the left half (rep gene) encodes 4 regulatory proteins with overlapping sequences which are known as Rep proteins (Rep78, Rep68, Rep52 and Rep40), because frame shift mutations at most locations within the open reading frame inhibit viral DNA replication (Hermonat et al., 1984). The Rep proteins are multi-functional DNA binding proteins. The functions of the Rep proteins in viral DNA replication include helicase activity and a site-specific, strand-specific endonuclease (nicking) activity (Ni et al., 1994).

AAV infects a broad spectrum of vertebrates from birds to humans, although in nature specific types are species specific (Berns, 1996). In humans AAV can infect a large variety of cells derived from different tissues. The infection of AAV is ubiquitous within the population with about 90% of adults being seropositive (Cukor et al., 1983). In spite of its omnipresence, AAV has never been associated with any human disease. In this sense, rAAV is the safest of the currently used gene therapy vectors.

Because of its propensity to establish latency and because it has not been implicated as a pathogen, AAV has been of considerable interest as a potential vector for human gene therapy (Flotte and Ferkol, 1997; Flotte and Carter, 1995). In general, rAAV vectors are produced by deleting the viral coding sequences and substituting the transgene of interest under control of a non-AAV promoter between the two AAV inverted terminal repeats (ITRs). When the rep and cap proteins are expressed in trans in Ad-infected cells, rAAV genomes can be efficiently packaged. Considerations in the development of AAV as a vector have included difficulties in attaining high vector titers and the limited insertional capacity (>5 kb). Although these issues can still be improved, recently developed packaging techniques for high titer and Ad-contamination free vectors, and strategies to overcome the packaging limitation, have dramatically impacted the applications of rAAV (Zolotukhin et al., 1999; Duan et al., 2000; Yan et al., 2000). Unlike adenovirus vectors, rAAV vectors are remarkably nonimmunogenic with little host response (Jooss et al., 1998; Song et al., 1998). In addition to the above unique features, rAAV have mediated long-term transgene expression in a wide variety of tissues, including muscle (Song et al., 1998; Kessler et al., 1996; Xiao et al., 1996; Clark et al., 1997; Snyder et al., 1997a), lung (Flotte et al., 1993), liver (Snyder et al., 1997b; Xiao et al., 1998; Song et al., 2001a; Xu et al., 2001), brain (Kaplitt et al., 1994) and eye (Flannery et al., 1997). Thus rAAV vectors appear to have significant advantages over other commonly used viral vectors.

Six serotypes of AAV have been cloned and sequenced. Of the six AAV serotypes, serotype 2 (AAV2) is the best-characterized and has been predominantly used in gene transfer studies. Membrane-associated heparan sulfate proteoglycan is the primary receptor for AAV type 2 (Summerford and Samulski, 1998). Human fibroblast growth factor receptor 1 and αᵥβ₅ integrin are co-receptors for AAV2 (Qing et al., 1999; Summerford et al., 1999). Serotypes 1 and 6 share >99% amino acid homology in their capsid proteins. Sequence analysis supports a recombination event between seroType I and 2. Comparison of the serotype capsid amino acid sequences suggests that serotypes, 1, 2, and 3 share homology across the three capsids in accord with heparan sulfate binding (Summerford and Samulski, 1998). In contrast, AAV type 4 and 5 are the most divergent of the six AAV serotypes, exhibiting only 60% homology to AAV2 or to each other. AAV4 and AAV5 require different sialic acid-containing glycoproteins for binding and transduction of target cells. The different tropisms of AAV serotypes provide opportunities to optimize the transduction efficiency in different target cells. Data showed that of the serotypes, AAV1 mediated the highest transgene expression in skeletal muscle and murine islets (Chao et al., 2000).

### 4.6 Promoters and Enhancers

Recombinant AAV vectors form important aspects of the present invention. The term “expression vector or construct” means any type of genetic construct containing a nucleic acid in which part or all of the nucleic acid encoding sequence is capable of being transcribed. In preferred embodiments, expression only includes transcription of the nucleic acid, for example, to generate a biologically-active serpin or cytokine polypeptide product from a transcribed gene.

Particularly useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

In preferred embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with an cytokine or serpin-encoding gene in its natural environment. Such promoters may include promoters normally associated with other genes, and/or promoters isolated from any bacterial, viral, eukaryotic, or mammalian cell.

Naturally, it will be important to employ a promoter that effectively directs the expression of the serpin or cytokine-encoding DNA segment in the cell type, organism, or even animal, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high-level expression of the introduced DNA segment, or the promoters may direct tissue- or cell-specific expression of the therapeutic constructs, such as, for example, an islet cell- or pancreas-specific promoter such as the insulin promoter.

At least one module in a promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the serpin or cytokine-polypeptide encoding nucleic acid segment in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter, such as a CMV or an HSV promoter. In certain aspects of the invention, β-actin, and in particular, chicken β-actin promoters have been shown to be particularly preferred for certain embodiments of the invention.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters that are well known in the art to achieve expression of a transgene is contemplated as well, provided that the levels of expression are sufficient for a given purpose. A variety of promoter elements have been described in Tables 1 and 2 that may be employed, in the context of the present invention, to regulate the expression of the present serpin or cytokine-encoding nucleic acid segments comprised within the recombinant AAV vectors of the present invention.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
ILLUSTRATIVE PROMOTER AND ENHANCER ELEMENTS

| PROMOTER/ENHANCER | REFERENCES |
|---|---|
| Immunoglobulin Heavy Chain | Banerji et al., 1983; Gilles et al., 1983; Grosschedl and |
| | Baltimore, 1985; Atchinson and Perry, 1986, 1987; Imler |
| | et al., 1987; Weinberger et al., 1984; Kiledjian et al., 1988; |
| | Porton et al.; 1990 |
| Immunoglobulin Light Chain | Queen and Baltimore, 1983; Picard and Schaffner, 1984 |
| T-Cell Receptor | Luria et al., 1987; Winoto and Baltimore, 1989; Redondo |
| | et al.; 1990 |
| HLA DQ a and DQ β | Sullivan and Peterlin, 1987 |
| β-Interferon | Goodbourn et al., 1986; Fujita et al., 1987; Goodbourn and |
| | Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick and Benfield, 1989; Johnson |
| | et al., 1989 |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987; Tronche et al., 1989, 1990 |
| α-Fetoprotein | Godbout et al., 1988; Campere and Tilghman, 1989 |
| t-Globin | Bodine and Ley, 1987; Perez-Stable and Constantini, 1990 |
| β-Globin | Trudel and Constantini, 1987 |
| e-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins (GRP94 | Chang et al., 1989 |
| and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh and Lockett, |
| | 1985; Firak and Subramanian, 1986; Herr and Clarke, 1986; |
| | Imbra and Karin, 1986; Kadesch and Berg, 1986; Wang and |
| | Calame, 1986; Ondek et al., 1987; Kuhl et al., 1987; |
| | Schaffner et al., 1988 |
| Polyoma | Swartzendruber and Lehman, 1975; Vasseur et al., 1980; |
| | Katinka et al., 1980, 1981; Tyndell et al., 1981; Dandolo |
| | et al., 1983; de Villiers et al., 1984; Hen et al., 1986; Satake |
| | et al., 1988; Campbell and Villarreal, 1988 |
| Retroviruses | Kriegler and Botchan, 1982, 1983; Levinson et al., 1982; |
| | Kriegler et al., 1983, 1984a, b, 1988; Bosze et al., 1986; |
| | Miksicek et al., 1986; Celander and Haseltine, 1987; |
| | Thiesen et al., 1988; Celander et al., 1988; Chol et al., 1988; |
| | Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; Hirochika et al., |
| | 1987; Stephens and Hentschel, 1987 |
| Hepatitis B Virus | Bulla and Siddiqui, 1986; Jameel and Siddiqui, 1986; Shaul |
| | and Ben-Levy, 1987; Spandau and Lee, 1988; Vannice and |
| | Levinson, 1988 |
| Human Immunodeficiency Virus | Muesing et al., 1987; Hauber and Cullan, 1988; Jakobovits |
| | et al., 1988; Feng and Holland, 1988; Takebe et al., 1988; |
| | Rosen et al., 1988; Berkhout et al., 1989; Laspia et al., |
| | 1989; Sharp and Marciniak, 1989; Braddock et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

TABLE 2
INDUCIBLE ELEMENTS

| ELEMENT | INDUCER | REFERENCES |
|---|---|---|
| MT II | Phorbol Ester (TFA) | Palmiter et al., 1982; Haslinger |
| | Heavy metals | and Karin, 1985; Searle et al., |
| | | 1985; Stuart et al., 1985; |
| | | Imagawa et al., 1987, Karin et al., |
| | | 1987; Angel et al., 1987b; |
| | | McNeall et al., 1989 |
| MMTV (mouse mammary | Glucocorticoids | Huang et al., 1981; Lee et al., |
| tumor virus) | | 1981; Majors and Varmus, 1983; |
| | | Chandler et al., 1983; Lee et al., |
| | | 1984; Ponta et al., 1985; Sakai |
| | | et al., 1988 |
| β-Interferon | poly(rI) × | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle Disease | |
| | Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | Ela, SV40 Large T Antigen | Taylor et al., 1989; Taylor and |
| | | Kingston, 1990a, b |
| Proliferin | Phorbol Ester-TPA | Mordacq and Linzer, 1989 |
| Tumor Necrosis Factor | FMA | Hensel et al., 1989 |
| Thyroid Stimulating Hormone | Thyroid Hormone | Chatterjee et al., 1989 |
| a Gene | | |

As used herein, the terms “engineered” and “recombinant” cells are intended to refer to a cell into which an exogenous DNA segment, such as DNA segment that leads to the transcription of a biologically-active serpin or cytokine polypeptide or a ribozyme specific for such a biologically-active serpin or cytokine polypeptide product, has been introduced. Therefore, engineered cells are distinguishable from naturally occurring cells, which do not contain a recombinantly introduced exogenous DNA segment. Engineered cells are thus cells having DNA segment introduced through the hand of man.

To express a biologically-active serpin or cytokine encoding gene in accordance with the present invention one would prepare an rAAV expression vector that comprises a biologically-active serpin or cytokine polypeptide-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. Particularly preferred recombinant vector constructs are those that comprise an rAAV vector. Such vectors are described in detail herein.

### 4.7 Pharmaceutical Compositions

In certain embodiments, the present invention concerns formulation of one or more of the rAAV compositions disclosed herein in pharmaceutically acceptable solutions for administration to a cell or an animal, either alone or in combination with one or more other modalities of therapy, and in particular, for therapy of the mammalian pancreas and the tissues and cells thereof, such as for example, pancreatic islet cells.

It will also be understood that, if desired, nucleic acid segments, RNA, DNA or PNA compositions that express one or more of the therapeutic gene products as disclosed herein may be administered in combination with other agents as well, such as, e.g., proteins or polypeptides or various pharmaceutically-active agents, including one or more systemic or localized administrations of serpin or cytokine polypeptides, biologically active fragments, or variants thereof. In fact, there is virtually no limit to other components that may also be included, given that the additional agents do not cause a significant adverse effect upon contact with the target cells or host tissues. The rAAV compositions may thus be delivered along with various other agents as required in the particular instance. Such compositions may be purified from host cells or other biological sources, or alternatively may be chemically synthesized as described herein. Likewise, such compositions may further comprise substituted or derivatized RNA, DNA, or PNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the AAV vector-based therapeutic constructs in suitably formulated pharmaceutical compositions disclosed herein either subcutaneously, intraocularly, intravitreally, parenterally, intravenously, intramuscularly, intrathecally, or even orally, intraperitoneally, or by nasal inhalation, including those modalities as described in U.S. Pat. No. 5,543,158; U.S. Pat. No. 5,641,515 and U.S. Pat. No. 5,399,363 (each specifically incorporated herein by reference in its entirety). Solutions of the active compounds as freebase or pharmacologically acceptable salts may be prepared in sterile water and may also suitably mixed with one or more surfactants, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active AAV vector-delivered serpin or cytokine-encoding polynucleotides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The AAV vector compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human, and in particular, when administered to the human pancreas, or cells or tissues thereof. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

### 4.8 Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the rAAV vector delivered gene therapy compositions of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically acceptable nanocapsule formulations of the AAV vector-based polynucleotide compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; Couvreur, 1988; zur Muhlen et al., 1998; Zambaux et al. 1998; Pinto-Alphandry et al., 1995 and U.S. Pat. No. 5,145,684, specifically incorporated herein by reference in its entirety).

### 4.9 Mutagenesis and Preparation of Modified Nucleotide Compositions

In certain embodiments, it may be desirable to prepared modified nucleotide compositions, such as, for example, in the generation of the nucleic acid segments that encode either parts of the AAV vector itself, or the promoter, or even the therapeutic gene delivered by such rAAV vectors. Various means exist in the art, and are routinely employed by the artisan to generate modified nucleotide compositions.

Site-specific mutagenesis is a technique useful in the preparation and testing of sequence variants by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector that includes within its sequence a DNA sequence encoding the desired ribozyme or other nucleic acid construct. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected nucleic acid sequences using site-directed mutagenesis is provided as a means of producing potentially useful species and is not meant to be limiting, as there are other ways in which sequence variants may be obtained. For example, recombinant vectors encoding the desired gene may be treated with mutagenic agents, such as hydroxylamine, to obtain sequence variants.

### 4.10 Nucleic Acid Amplification

In certain embodiments, it may be necessary to employ one or more nucleic acid amplification techniques to produce the nucleic acid segments of the present invention. Various methods are well-known to artisans in the field, including for example, those techniques described herein:

Nucleic acid, used as a template for amplification, may be isolated from cells contained in the biological sample according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to the ribozymes or conserved flanking regions are contacted with the isolated nucleic acid under conditions that permit selective hybridization. The term “primer”, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (e.g., Affymax technology).

A number of template dependent processes are available to amplify the marker sequences present in a given template sample. One of the best-known amplification methods is the polymerase chain reaction (referred to as PCR™), which is described in detail in U.S. Pat. No. 4,683,195, U.S. Pat. No. 4,683,202 and U.S. Pat. No. 4,800,159 (each of which is incorporated herein by reference in its entirety).

Briefly, in PCR™, two primer sequences are prepared that are complementary to regions on opposite complementary strands of the marker sequence. An excess of deoxynucleoside triphosphates is added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in Int. Pat. Appl. Publ. No. WO 90/07641 (specifically incorporated herein by reference). Polymerase chain reaction methodologies are well known in the art.

Another method for amplification is the ligase chain reaction (“LCR”), disclosed in EPA No. 320 308, and incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qβ Replicase (QβR), described in Int. Pat. Appl. No. PCT/US87/00880, incorporated herein by reference, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]-triphosphates in one strand of a restriction site may also be useful in the amplification of nucleic acids in the present invention.

Strand Displacement Amplification (SDA), described in U.S. Pat. Nos. 5,455,166, 5,648,211, 5,712,124 and 5,744,311, each incorporated herein by reference, is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in Int. Pat. Appl. No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR Gingeras et al., Int. Pat. Appl. Publ. No. WO 88/10315, incorporated herein by reference. In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPA No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., Int. Pat. Appl. Publ. No. WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990, specifically incorporated herein by reference).

Methods based on ligation of two (or more) oligonucleotides in the presence of nucleic acid having the sequence of the resulting “di-oligonucleotide,” thereby amplifying the di-oligonucleotide, may also be used in the amplification step of the present invention.

Following any amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods (see e.g., Sambrook et al., 1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography.

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by Southern blotting and hybridization with a labeled probe. The techniques involved in Southern blotting are well known to those of skill in the art and can be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

### 4.11 Methods of Nucleic Acid Delivery and DNA Transfection

In certain embodiments, it is contemplated that one or more RNA, DNA, PNAs and/or substituted polynucleotide compositions disclosed herein will be used to transfect an appropriate host cell. Technology for introduction of PNAs, RNAs, and DNAs into cells is well known to those of skill in the art.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Wong and Neumann, 1982; Fromm et al., 1985; Tur-Kaspa et al., 1986; Potter et al., 1984; Suzuki et al., 1998; Vanbever et al., 1998), direct microinjection (Capecchi, 1980; Harland and Weintraub, 1985), DNA-loaded liposomes (Nicolau and Sene, 1982; Fraley et al., 1979; Takakura, 1998) and lipofectamine-DNA complexes, cell sonication (Fechheimer et al., 1987), gene bombardment using high velocity microprojectiles (Yang et al., 1990; Klein et al., 1992), and receptor-mediated transfection (Curiel et al., 1991; Wagner et al., 1992; Wu and Wu, 1987; Wu and Wu, 1988). Some of these techniques may be successfully adapted for in vivo or ex vivo use.

### 4.12 Expression Vectors

The present invention contemplates a variety of AAV-based expression systems, and vectors. In one embodiment the preferred AAV expression vectors comprise at least a first nucleic acid segment that encodes a therapeutic antisense molecule. In another embodiment, a promoter is operatively linked to a sequence region that encodes a functional mRNA, a tRNA, a ribozyme or an antisense RNA.

As used herein, the term “operatively linked” means that a promoter is connected to a functional RNA in such a way that the transcription of that functional RNA is controlled and regulated by that promoter. Means for operatively linking a promoter to a functional RNA are well known in the art.

The choice of which expression vector and ultimately to which promoter a polypeptide coding region is operatively linked depend directly on the functional properties desired, e.g., the location and timing of protein expression, and the host cell to be transformed. These are well known limitations inherent in the art of constructing recombinant DNA molecules. However, a vector useful in practicing the present invention is capable of directing the expression of the functional RNA to which it is operatively linked.

RNA polymerase transcribes a coding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA).

A variety of methods have been developed to operatively link DNA to vectors via complementary cohesive termini or blunt ends. For instance, complementary homopolymer tracts can be added to the DNA segment to be inserted and to the vector DNA. The vector and DNA segment are then joined by hydrogen bonding between the complementary homopolymeric tails to form recombinant DNA molecules.

### 4.13 Biological Functional Equivalents

Modification and changes to the structure of the polynucleotides and polypeptides of wild-type rAAV vectors to provide the improved rAAV virions as described in the present invention to obtain functional viral vectors that possess desirable characteristics, particularly with respect to improved delivery of therapeutic gene constructs to selected mammalian cell, tissues, and organs for the treatment, prevention, and prophylaxis of various diseases and disorders, as well as means for the amelioration of symptoms of such diseases, and to facilitate the expression of exogenous therapeutic and/or prophylactic polypeptides of interest via rAAV vector-mediated gene therapy. As mentioned above, one of the key aspects of the present invention is the creation of one or more mutations into specific polynucleotide sequences that encode one or more of the therapeutic agents encoded by the disclosed rAAV constructs. In certain circumstances, the resulting polypeptide sequence is altered by these mutations, or in other cases, the sequence of the polypeptide is unchanged by one or more mutations in the encoding polynucleotide to produce modified vectors with improved properties for effecting gene therapy in mammalian systems.

When it is desirable to alter the amino acid sequence of a polypeptide to create an equivalent, or even an improved, second-generation molecule, the amino acid changes may be achieved by changing one or more of the codons of the encoding DNA sequence, according to Table 3.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the polynucleotide sequences disclosed herein, without appreciable loss of their biological utility or activity.

TABLE 3

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4), threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those that are within ±1 are particularly preferred, and those within ±0.5 are even more particularly preferred. It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take several of the foregoing characteristics into consideration are well known to those of skill in the art and include: arginine and lysine; glutamate and aspartate; serine and threonine; glutamine and asparagine; and valine, leucine and isoleucine.

### 4.14 Therapeutic and Diagnostic Kits

The invention also encompasses one or more disclosed rAAV compositions together with one or more pharmaceutically-acceptable excipients, carriers, diluents, adjuvants, and/or other components, as may be employed in the formulation of particular rAAV-polynucleotide delivery formulations, and in the preparation of therapeutic agents for administration to a mammal, and in particularly, to a human, for one or more of the conditions described herein. In particular, such kits may comprise one or more of the disclosed rAAV compositions in combination with instructions for using the viral vector in the treatment of such disorders in a mammal, and may typically further include containers prepared for convenient commercial packaging.

As such, preferred animals for administration of the pharmaceutical compositions disclosed herein include mammals, and particularly humans. Other preferred animals include primates, simians, murines, bovines, ovines, lupines, vulpines, equines, porcines, canines, and felines as well as any other mammalian species commonly considered pets, livestock, or commercially relevant animal species. The composition may include partially or significantly purified rAAV compositions, either alone, or in combination with one or more additional active ingredients, which may be obtained from natural or recombinant sources, or which may be obtainable naturally or either chemically synthesized, or alternatively produced in vitro from recombinant host cells expressing DNA segments encoding such additional active ingredients.

Therapeutic kits may also be prepared that comprise at least one of the compositions disclosed herein and instructions for using the composition as a therapeutic agent. The container means for such kits may typically comprise at least one vial, test tube, flask, bottle, syringe or other container means, into which the disclosed rAAV composition(s) may be placed, and preferably suitably aliquoted. Where a second therapeutic composition is also provided, the kit may also contain a second distinct container means into which this second composition may be placed. Alternatively, the plurality of biologically-active therapeutic compositions may be prepared in a single pharmaceutical composition, and may be packaged in a single container means, such as a vial, flask, syringe, bottle, or other suitable single container means. The kits of the present invention will also typically include a means for containing the vial(s) in close confinement for commercial sale, such as, e.g., injection or blow-molded plastic containers into which the desired vial(s) are retained.

### 4.15 Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from native sources, chemically synthesized, modified, or otherwise prepared in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

A, an: In accordance with long standing patent law convention, the words “a” and “an” when used in this application, including the claims, denotes “one or more”.

Expression: The combination of intracellular processes, including transcription and translation undergone by a polynucleotide such as a structural gene to synthesize the encoded peptide or polypeptide.

Promoter: a term used to generally describe the region or regions of a nucleic acid sequence that regulates transcription.

Regulatory Element: a term used to generally describe the region or regions of a nucleic acid sequence that regulates transcription.

Structural gene: A gene or sequence region that is expressed to produce an encoded peptide or polypeptide.

Transformation: A process of introducing an exogenous polynucleotide sequence (e.g., a vector, a recombinant DNA or RNA molecule) into a host cell or protoplast in which that exogenous nucleic acid segment is incorporated into at least a first chromosome or is capable of autonomous replication within the transformed host cell. Transfection, electroporation, and naked nucleic acid uptake all represent examples of techniques used to transform a host cell with one or more polynucleotides.

Transformed cell: A host cell whose nucleic acid complement has been altered by the introduction of one or more exogenous polynucleotides into that cell.

Transgenic cell: Any cell derived or regenerated from a transformed cell or derived from a transgenic cell, or from the progeny or offspring of any generation of such a transformed host cell.

Vector: A nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

The terms “substantially corresponds to”, “substantially homologous”, or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s) which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted on the basis of known consensus sequence motifs, or by other methods known to those of skill in the art.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than 1, 2, 3, 4, 5, 6, 7, 8, 9, or 10 base mismatches throughout the complementary portion of the sequence. In many instances, it may be desirable for the sequences to be exact matches, i.e. be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary oligonucleotide sequences will be greater than about 80 percent complementary (or ‘% exact-match’) to the corresponding mRNA target sequence to which the oligonucleotide specifically binds, and will, more preferably be greater than about 85 percent complementary to the corresponding mRNA target sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 90 percent complementary to the corresponding mRNA target sequence to which the oligonucleotide specifically binds, and may in certain embodiments be greater than about 95 percent complementary to the corresponding mRNA target sequence to which the oligonucleotide specifically binds, and even up to and including 96%, 97%, 98%, 99%, and even 100% exact match complementary to all or a portion of the target mRNA to which the designed oligonucleotide specifically binds.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

### 5. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

### Ex Vivo Transduction of Murine Islets with rAAV Vectors Expressing Elafin

To test the local effects of elafin on protecting transplanted islets, donor islets are ex vivo transduced with rAAV-CB-Elafin vector, followed by a series of in vitro and in vivo assessments pertaining to metabolic, immunologic and pathologic function. Islets from various donor strains are then transplanted under the kidney capsule in specific groups of animals. Islet produced elafin will prevent the islets from recurrent autoimmunity and alloimmune rejection

The physiological replacement of insulin producing cells afforded by islet cell transplantation represents an exciting alternative to exogenous insulin administration as it offers excellent metabolic control (Hering et al., 1993; Kenyon et al., 1996; Rosenberg, 1998; Cretin et al., 1998). Previous clinical trials have established that long-term (i.e., >7 years) function of transplanted islets can be observed in selected recipients (Alejandro et al., 1997; Warnock et al., 1991; Scharp et al., 1991; Socci et al., 1991). However, for a vast majority of individuals, islet transplantation remains unsuccessful, with a substantial percentage of recipients losing graft function a short time after transplantation. Among the likely reasons for this relative lack of clinical success is the action of several concurrent mechanisms, including rejection, recurrence of anti-islet cell autoimmunity and non-specific islet loss immediately after transplantation due to perturbation of the graft microenvironment (inflammation, ischemia/reperfusion) (Kaufman et al., 1990; Weir et al., 1990; Stevens et al., 1994; Nussler et al., 1992; Bottino et al., 1998). For islet transplantation to become a clinical reality, a need exists to devise strategies of immunosuppression/immunomodulation that are substantially different from those presently utilized. The reasons for this need include: the apparent inadequacy of currently available immunosuppressive agents to reproducibly promote long-term islet graft survival; the direct toxic effects of these drugs on islet function; and serious unwanted side effects linked to chronic immunosuppression (Hering et al., 1993; Kenyon et al., 1996; Rosenberg, 1998; Cretin et al., 1998; Penn, 1989; Dunn, 1990; Jindal, 1994; Hahn et al., 1986; Hirano et al., 1992; Venkatesan et al., 1987; Guo et al., 1997). Indeed, conventional immunosuppressive agents routinely used in islet transplant patients (e.g., cyclosporine, FK506, and steroids) are characterized by intrinsic diabetogenic effects imposing a two- to three-fold increase in the metabolic demand of islet cells (Jindal, 1994; Hahn et al., 1986; Hirano et al., 1992; Venkatesan et al., 1987; Guo et al., 1997).

5.1.1 Diabetic Recipient Animals Intramuscularly Injected with rAAV Vectors Before Islets Transplantation

To test the systemic effects of these serpins on protecting transplanted islets, untransduced islets from various donor strains are transplanted to diabetic recipients, which are intramuscularly injected with rAAV-CB-Elafin vector. These results demonstrate that over-expression of elafin in the circulation alters the immune response and protects transplanted islets.

### 5.1.2 Human Islets Secrete AAT

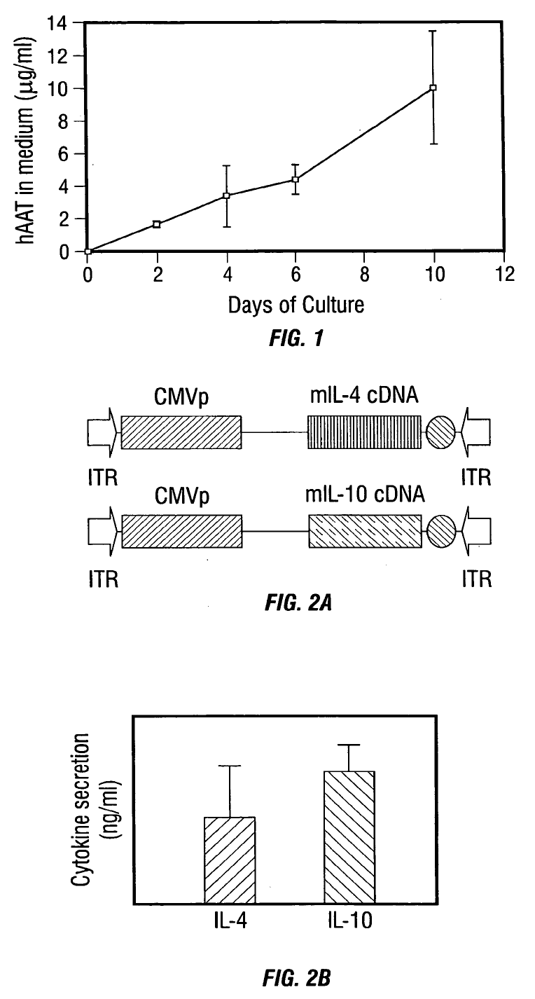

Although AAT is primarily secreted from hepatocytes, other cells including neutrophils, monocytes, macrophages, alveolar macrophages, intestinal epithelial cells, carcinoma cells and the cornea also express AAT (Ray et al., 1977; Geboes et al., 1982; Keppler et al., 1996; Boskovic and Twining, 1998). It has been shown that human neutrophils, monocytes, alveolar macrophages can increase expression of AAT in response to inflammatory mediators, such as IL-6, bacterial lipopolysaccharide, and in response to AAT itself when complexed with neutrophil elastase (Perlmutter and Punsal, 1988; Knoell et al., 1998). Local expression of AAT may play an important role in anti-inflammatory effects. In order to observe whether AAT is expressed in islet cells, human islets were cultured. The medium was sampled for detection of AAT. High levels of AAT (in comparison to other cell types tested) were detected by ELISA (FIG. 1). The expression of AAT in islets provides evidence that AAT may play a role in protection of islets.

5.1.3 Efficient Transduction of rAAV Vector to Islet Cells

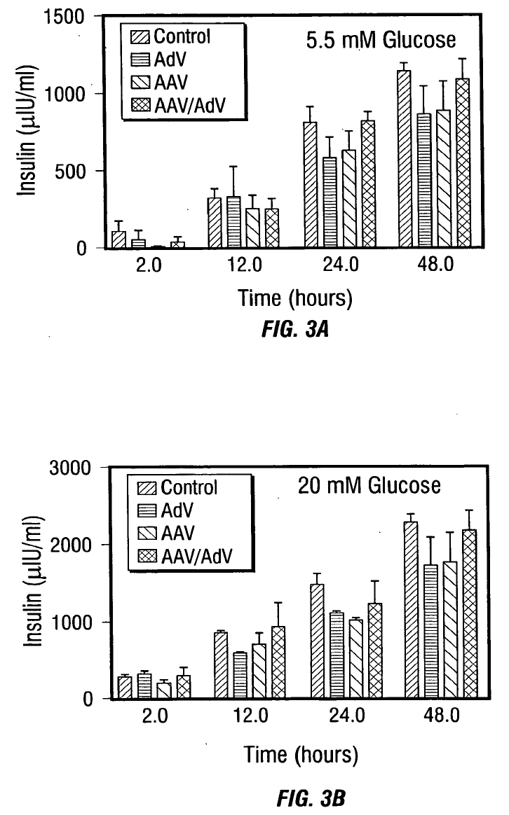

The ability to transfer immunoregulatory, cytoprotective, or anti-apoptotic genes into pancreatic islet cells may allow enhanced post-transplantation survival of islet allografts and inhibition of recurrent autoimmune destruction of these cells in Type I diabetes. However, transient transgene expression and the tendency to induce host inflammatory responses have limited previous gene delivery studies using viral transfer vectors. It has been demonstrated that rAAV2 vector that can overcome these limitations, effectively transduces both human and murine pancreatic islet cells with reporter genes as well as potentially important immunoregulatory cytokine genes (interleukin-4, interleukin-10), although a very high multiplicity of infection was required (FIG. 2). The rAAV-delivered transgenes did not interfere with islet cell insulin production and were expressed in both β- and non-β-cells (FIG. 3). These results indicate that rAAV is a useful tool to deliver therapeutic genes for modulating immune responses against islet cells and markedly enhancing long-term graft survival (Flotte et al., 2001).

5.1.4 AAV Serotype I Mediates Highest Level of hAAT Expression in Mouse Islets

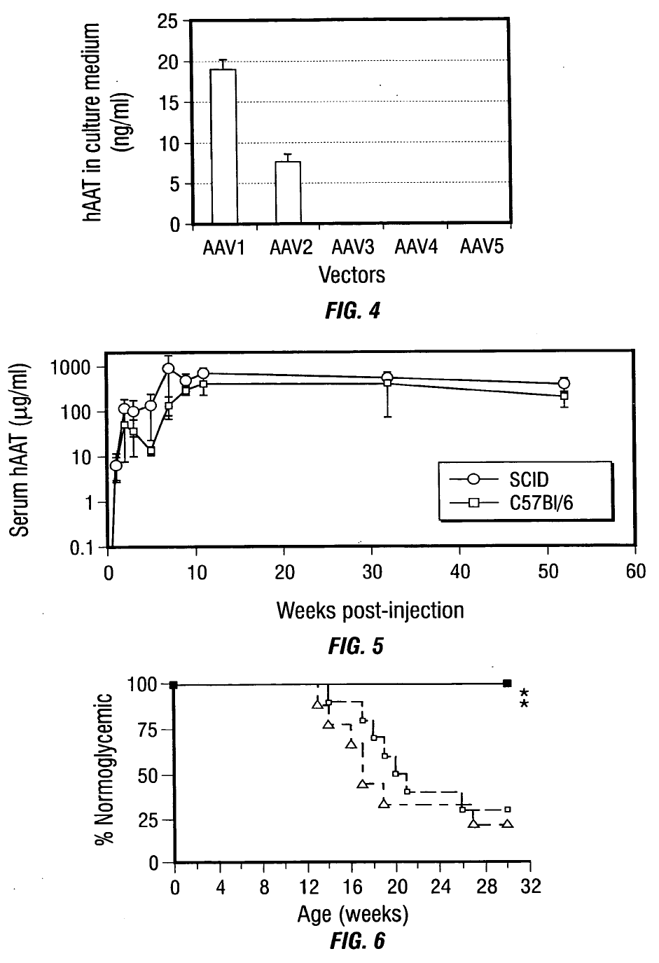

In order to increase the transduction efficiency of rAAV vector to mouse islets, rAAV-CB-hAAT vector into seroType I, 2, 3, 4 and 5 have been recently packaged. The same dose of these vectors was used to infect mouse islets (1×10⁹ particles/80 islets). FIG. 3 shows that rAAV1-CB-hAAT mediated highest secretion of hAAT from mouse islets (FIG. 4).

5.1.5 AAV Vector Mediated Long Term and High Level of hAAT Secretion from Muscle

To test the feasibility of using skeletal muscle as platform for rAAV mediated hAAT gene transfer, cohorts of C57Bl/6 (n=3) and C57Bl/6-SCID (SCID) mice (n=3) were injected intramuscularly with 1.4×10¹³ DNase-resistant particles (4×10¹⁰ infectious units) of an rAAV vector (C-AT) expressing human α-1-antitrypsin (hAAT) from the CMV immediate early promoter. These mice expressed and secreted high levels of hAAT into the serum (400 to 800 μg/ml). Transgene expression in both strains has remained at levels over 200 to 400 μg/ml for 52 weeks post-injection (FIG. 5) (Song et al., 1998; Song et al., 2001b).

5.1.6 Experience of rAAV Mediated Gene Therapy for Type I Diabetes

The development of spontaneous autoimmune diabetes in NOD mice provides for their use as a model of human Type I diabetes. To test the feasibility of muscle directed gene therapy to prevent Type I diabetes, recombinant adeno-associated virus (rAAV) vectors containing murine cDNAs for the immunomodulatory cytokines IL-4 or IL-10 were developed (Goudy et al., 2001). Female NOD mice at 4 wk of age were intramuscularly injected with purified vector preparations of rAAV-IL-4 and/or rAAV-IL-10. rAAV-IL-10 transduction completely abrogated the development of diabetes (0/10; 0% incidence at 30 wk) (FIG. 6). In addition, mice receiving the combination therapy of both rAAV-IL-10 and rAAV-IL-4 were also protected from the disease (0/10; 0%). This protection was associated with rAAV-IL-10 only as rAAV-IL-4 treated animals did not display a significant delay in the kinetics of disease development (FIG. 6) nor did they demonstrate long-term differences in disease frequency (7/10; 70%) when compared to control animals (8/10; 80%).

A key feature of Type I diabetes in NOD mice is the infiltration of the endocrine pancreas with inflammatory cells. In contrast to a normal islet (stage 0 insulitis), the mildest form of inflammation is infiltration with inflammatory cells (dendritic cells, macrophages, T and B cells) around the perivascular duct and peri-islet regions of the islets of Langerhans (“peri-insulitis,” stage 1 insulitis). This peri-insulitis process in NOD mice normally begins when the animals are 4 to 6 wk of age, and is followed by an increase in the number of affected islets, a progressive increase in the quantity of intra-islet inflammatory cell accumulation (stage 2 and 3), and the selective destruction of insulin-producing islet β cells (loss of insulin content). To examine the immunomodulatory effect of rAAV-cytokine gene therapy on the insulitis lesion before the period of developing overt clinical disease, insulitis was monitored on a separate series of animals injected with rAAV vectors and sacrificed at 10 to 14 wk of age. Cytokine transduction of NOD mice with rAAV-IL-10 and to a lesser extent with rAAV-IL-4 effectively reduced the quantitative parameters of insulitis in recipient animals. Specifically, pancreatic sections from all mice contained islets free of inflammation as well as islets which demonstrated moderate to severe insulitis. However, the percentage of islets affected by severe insulitis was far less in the pancreata from rAAV-IL-10 NOD mice than in control animals, with rAAV-IL-4 treated mice forming an intermediate group. Furthermore, insulin content appeared retained and at higher levels in islets of rAAV-IL-10 transduced mice compared to those from the rAAV-IL-4 or control group mice.

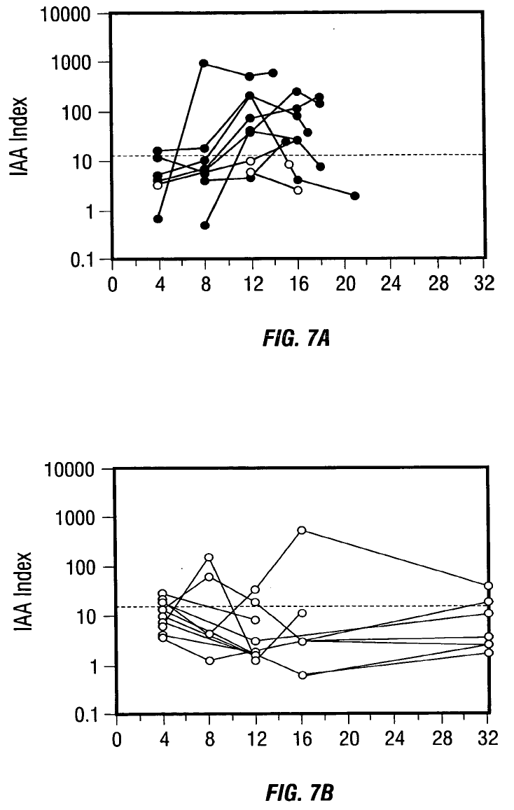

A recent report suggested that insulin autoantibodies (IAA) provide an excellent predictor of future development of Type I diabetes in NOD mice since a majority of animals developing disease possessed this marker by 12 wk of age (Yu et al., 2000). Hence, longitudinal analysis of IAA in rAAV-IL-10, rAAV-IL-4, and control animals was performed. As expected, serum from a minority of animals at 4 wk of age were IAA positive (FIG. 7A and FIG. 7B). In saline controls, both longitudinal analysis (FIG. 7A) and life table analysis (FIG. 6) of individual animals suggested a strong association between IAA development and the formation of Type I diabetes. Indeed, all saline treated animals developing Type I diabetes developed IAA by 16 wk of age. Interestingly, the effect of rAAV-IL-10 treatment appeared to involve a reduction in IAA index (FIG. 7B) in the period beyond 8 wk of age in nearly all animals. Hence, it is possible that the protection from Type I diabetes observed in rAAV-IL-10 treated mice resulted to some degree from the attenuation of islet autoantigen specific immunity in vivo.

These results indicate the utility for rAAV, a vector with advantages for therapeutic gene delivery, to transfer immunoregulatory cytokines capable of preventing Type I diabetes. In addition, these studies provide evidence for using immunoregulatory agents delivered by rAAV to modulate a variety of disorders associated with deleterious immune responses including allergic reactions, transplantation rejection, immunodeficiencies, and autoimmune disorders.

### 5.1.7 AAT Gene Transfer Prevents Type I Diabetes

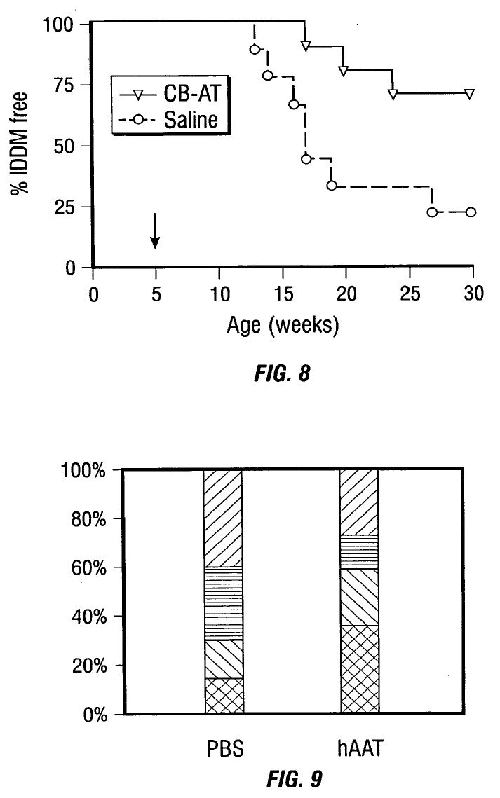

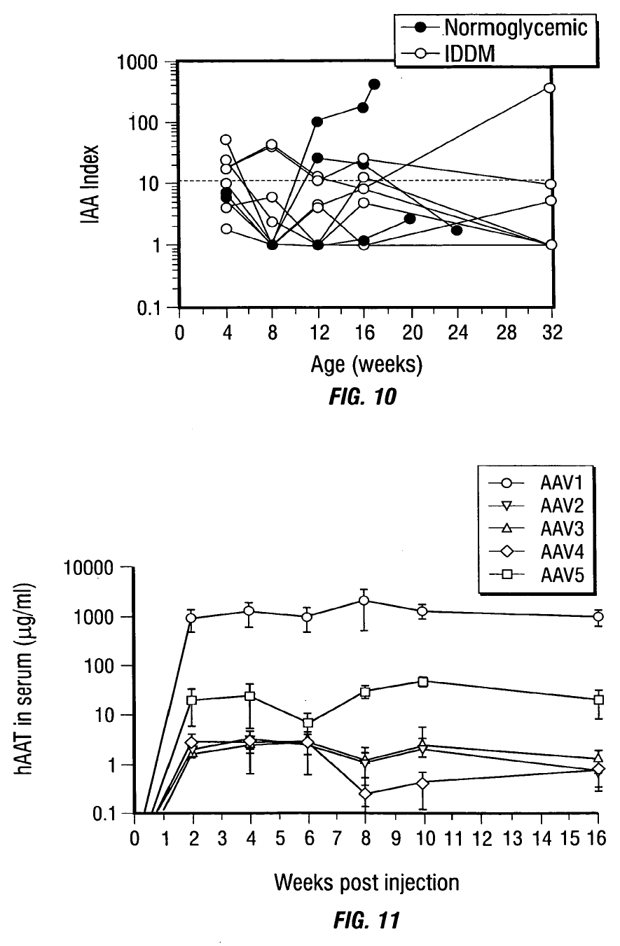

To test the effect of hAAT in preventing Type I diabetes, female NOD mice were intra-muscularly injected with rAAV2-CB-AT vector (1×10¹⁰ i.u./mouse, n=10) at 4 wk of age. FIG. 8 shows that muscle expressed hAAT prevents diabetes development (70% animals are Type I diabetes free at 30 wk of age). Insulitis was also monitored on a separate series of animals (n=10) injected with rAAV2-CB-AT vector and sacrificed at 10-16 wk of age. FIG. 9 shows that gene delivery of hAAT markedly reduced insulitis. Similar to IL-10 gene transfer, hAAT gene transfer also lowers the serum levels of insulin autoantibodies (FIG. 10) compare to that of control animals (FIG. 7A). Transgene expression of hAAT was observed at the injection site by immunostaining, while no infiltrations were observed at injection sites.

### 5.1.8 AAV Serotype I Mediated 1000-Fold Higher Transduction Efficiency in Skeletal Muscle

In order to optimize the transduction efficiency of rAAV vectors to muscle, five serotypes of rAAV-CB-hAAT vector (Type I, 2, 3, 4 and 5) were separately injected into groups of C57bl/6 mice (1×10¹¹ particles/mouse). FIG. 11 shows that AAV1 mediates 1000-fold higher hAAT expression than AAV2. These results were consistent with that previously observed, and made it possible to achieve therapeutic levels of human AAT expression in large animal model or humans, in which more transgene product is required due to the body weight. More importantly, AAV1 vector with high transduction efficiency may enable us to scale up our experiments (more animals per treatment group, or more treatment groups), or to further increase the transgene expressions. All of these advantages may be helpful for this proposed study.

5.1.9 Ex Vivo Transduction of Islets with rAAV Vectors Expressing Elafin, Prior to Transplantation

Unfortunately, pilot clinical trials of allogenic islet transplantation in Type I diabetic patients have resulted in a low rate of graft function. A multiplicity of factors can contribute to this poor outcome including rejection, recurrence of anti-islet cell autoimmunity, and non-specific islet loss immediately after transplantation due to perturbation of the graft microenvironment (inflammation, ischemia/reperfusion) (Kaufman et al., 1990; Weir et al., 1990; Stevens et al., 1994; Nussler et al., 1992; Bottino et al., 1998). One potential approach to enhance islet transplantation is to engineer islet cells before transplantation to be more resistant to immune destruction and inflammation. Serpins, AAT and elafin have been shown to have anti-inflammatory and immunoregulatory properties. Given the efficient transduction of islet by rAAV1 vector, gene transfer of these serpins to islets may provide high potential for preventing islet rejection.

### 5.1.10 Experimental Design

These experiments are designed to investigate whether local (islet) production of AAT or elafin will impart protection from recurrent Type I diabetes. Freshly isolated mouse islets from different strains will be transduced (400 islets per animal, 1×10⁷ particles/islet equivalent) with rAAV1-CB-AAT or rAAV1-CB-Elafin. The transduced islets will be transplanted to groups of diabetic mice (n=10). The rAAV1-mutant AAT, and PBS will serve as controls. Selected donor/recipient pairs are outlined in Table 1. Graft survival will be calculated as the number of days before diabetes recurrence.

TABLE 1

| | Type of | |
|---|---|---|
| Donor to recipient | transplantation | Objectives |
| C57BL/6 into Nude | Allogenic | To monitor islet cell function |
| (strep) | | in diabetic immuno-deficient |
| | | environment. |
| C57BL/6 into NOD | Allo/Autoimm. | To test the protection of |
| (female) | | transduced islets from recurrence |
| | | of Type I diabetes after allogenic |
| | | transplantation. |
| NOD (male) into | Syng./Autoimm. | To test the protection of |
| | | transduced islets |
| NOD (female) | | from recurrence of Type I |
| | | diabetes after syngeneic |
| | | transplantation. |
| C57BL/6 into NOD | Allogenic | To test the protection of |
| (male/strep) | | transduced islets from |
| | | allogenic rejection. |

Four separate transplant combinations will be used. In the first set of experiments, islet from C57bl/6 donor will be transplanted to diabetic nude (T cell immunodeficient) mice. Diabetes will be induced by the administration of streptozotocin (220 mg/kg I.V. once). Analysis of islet function is obtained by assessing blood glucose profiles. This series of experiments will be of value in ascertaining that delivery of genes coding for selected immunomodulatory serpin does not negatively influence the functional performance of the islets.

In the second experimental combination, spontaneously diabetic female NOD mice will receive a fully H-2-mismatched islet graft from C57BL/6 donors under the kidney capsule. This combination will allow the study of the efficacy of gene delivery in preventing/delaying islet graft loss due to the simultaneous occurrence of allogenic graft rejection and recurrence of autoimmunity. This donor recipient combination is most relevant to the situation encountered in the human setting of islet transplantation, where a patient with an underlying autoimmune disorder receives islets from a largely incompatible donor.

In the third set of studies, spontaneously diabetic female NOD mice will be transplanted with syngeneic islets obtained from young (6-7-wk old) male NOD. This donor-recipient combination allows to pinpoint the role of recurrent autoimmunity, in the absence of confounding allorecognition phenomena, on islet graft loss, and the efficacy of gene delivery in preventing it.

Lastly, a complementary set of experiments will be performed using chemically induced diabetic male NOD mice as recipients, and C57BL/6 mice as donors. This donor-recipient combination is most relevant to complement experimental data obtained in the former group, since it provides information on allorejection in the NOD strain, with little influence exerted by autoimmunity.

### 5.1.11 Vector Production

Data has shown that both human and murine islets could be transduced by rAAV vectors. Of the five serotypes, rAAV1-CB-AAT mediated the highest expression of hAAT in murine islets and in skeletal muscle. Therefore, seroType I (pseudo-Type I using AAV2-ITRs and AAV1 capsid proteins) AAV vectors are used for both islet transduction and intramuscular injection.

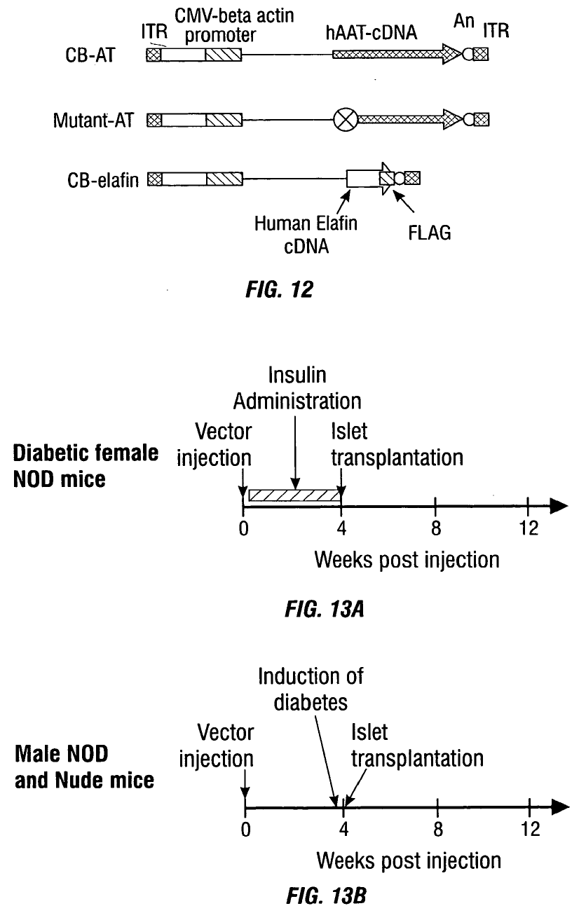

Plasmid CB-AT (FIG. 12), in which hAAT cDNA driven by CMV enhancer and chicken β-actin promoter is between full-length AAV2 ITRs, has been previously described (Song et al., 2001a; Xu et al., 2001). Plasmid CB-Elafin has been generated from pCB-AT by replacing hAAT cDNA (at 5′-XbaI and 3′-NotI sites) with a modified elafin cDNA fragment. The modified human elafin cDNA fragment contains entire elafin coding sequences with a insert of 30 bp sequences for a FLAG tag (10 AA) right before the stop codon. This fragment was amplified by PCR™ from a plasmid (pHZ7) with primers contain XbaI or NotI site, respectively. Both in vitro and in vivo studies confirmed that this C-terminus FLAG-tagged elafin construct produced a stable, and functional gene product (Hermonat and Muzyczka, 1984). The control vector (mutant-AAT) plasmid has been generated from C-AT (similar to PCB-AT) by deletion of the signal sequences (between BamHI sites). This deletion also creates a frame shift. In vitro transfection showed no hAAT was detected either in cell lysate or in culture medium.

To package rAAV1 vector, vector plasmid and the helper plasmid (pXYZ1), which contains AAV1 capsid and rep genes and adenovirus helper genes, are co-transfected into 293 cells. Cells are harvested and disrupted by freeze-thaw lysis to release virions that are purified by iodixanol gradient ultracentrifugation (Zolotukhin et al., 1999). The physical titers of vector preparations are assessed by quantitative dot-blot analysis. All vector preparations lack any detectable wtAAV by either physical particle or infectious unit measurement.

### 5.1.12 Islet Isolation

Pancreatic islet cells from various donor strains are isolated as previously described (Linetsky et al., 1997; Linetsky et al., 1998). Briefly, after intraductal injection of a solution containing Liberase®, multiple donor pancreata (in rodents) are loaded into a 50 ml digestion chamber. The digestion apparatus allows the islets to be progressively released during a continuous digestion process that involves a moderate mechanical component exerted by the movement of intrachamber glass beads (Ricordi et al., 1988). Purification of the final islet preparation is obtained by centrifugation on discontinuous Eurocollins-Ficoll gradients. Islets are maintained in RPMI-1640 with 10% fetal bovine serum; 5% CO₂, 24° C., until used (within 48 h).

### 5.1.13 Ex Vivo Transduction and Detection of Transgene Expression

Fresh intact islets are transduced with rAAV1-CB-AAT, rAAV1-CB-Elafin, or both vectors (1×10⁷ particle/islet for each vector) and cultured in RPMI-1640 with 10% fetal bovine serum; 5% CO₂, 24° C. for 12 hr prior transplantation. To assess transgene expression and the effect of the rAAV1 vector transduction, sample islets (50 islets/treatment) are cultured for 4 days. Human AAT and elafin in culture media are detected by ELISA or Western blot respectively. Insulin secretion is measured using commercial kits (Mercodia, Minneapolis, Minn.).

### 5.1.14 Islet Transplantation

NOD mice were purchased from Taconic Farms (Germantown, N.Y.). C57BL/6 and nude mice were purchased from the Jackson Laboratories (Bar Harbor, Me.). Female NOD mice were obtained at 8-10 weeks of age and monitored for blood glucose until they became diabetic. They are then utilized as islet recipients after at least two consecutive non-fasting blood glucose readings above 250 mg/dl. Islets of Langerhans are obtained from either 12-week-old C57BL/6 males or from 6-8 week old NOD males. Young NOD males are also used in selected studies, as recipients of allogenic B6 islet transplants.

Nude mice and male NOD mice are rendered diabetic via a single intravenous injection of 220 mg/kg streptozotocin (Like and Rossini, 1976; Rossini et al., 1977) (Sigma, St Louis, Mo.) freshly dissolved in citrate buffer. Diabetes occurrence is defined as two consecutive non-fasting blood glucose readings above 250 mg/dl. Only animals with blood glucose levels over 350 mg/dl at the time of the transplant are used as recipients.

Immediately prior to transplantation, islets are divided in aliquots of 700 IEQ per recipient. Under general anesthesia induced by methoxyflurane (Metofane, Schering-Plough Animal Health, Atlanta, Ga.), a left lobotomy is performed and the left kidney exteriorized and exposed. A breach is made in the kidney capsule and a polyethylene catheter will be introduced through the breach and advanced in the subcapsular space to the opposite pole of the kidney. Islets are at this time slowly and gently injected and allowed to spread at the pole. The catheter is then retrieved with care to avoid leaking of the transplanted islets. The opening is cauterized, and the kidney repositioned. Suture of muscle and skin follows.

### 5.1.15 Immunosuppression

Transient immunosuppression are administered to the recipient animals (with the exception of nude mice) to allow the survival of transplanted islet during the first three weeks following transplant, and permit expression of the delivered genes. A control group receives identical islet grafts in the absence of immunosuppression. Blockade of the T-lymphocyte costimulatory molecule CD154 (also known as CD 40 Ligand) may also be employed. First, an induction dose of 20 mg/kg of anti-CD154 ab (MR1) is administered intraperitoneally on days −1, 0 and 3, day 0 being the day of transplantation. An identical maintenance dose (20 mg/kg) is then administered on day 7, and every 7 days thereafter until day 21.

### 5.1.16 Graft Survival Analysis

Blood glucose levels are measured daily after transplantation on whole blood samples collected from the tail vein using a strip glucometer (Elite, Bayer). Graft survival is calculated as the number of days before diabetes recurrence. The day of diabetes recurrence is defined as the first of 2 consecutive days of non-fasting blood glucose above 250 mg/ml. Animals are sacrificed after confirmation of diabetes recurrence and the transplanted kidneys are harvested for histology. Long term survival of the graft is defined as good blood glucose control persisting for more than 120 days. Long term surviving grafts are explanted (nephrectomy) to confirm prompt return to hyperglycemia, and for in vitro analysis of the explanted tissue.

5.1.17 rAAV Injection of Diabetic Recipient Animals Before Islets Transplantation

It is clear that autoimmune diseases, including Type I diabetes, result from dysregulation of the autoimmunity. Therefore, using immunoregulatory agents to modulate disorders associated with immune responses including allergic reactions, transplantation rejection, immunodeficiencies and autoimmune disorders provides an approach for the prevention and treatment of these diseases. In addition, data have shown that rAAV mediated AAT gene transfer prevented Type I diabetes (70% survival in CB-AT treatment group vs. 20% survival in control group), and decreased insulitis and IAA levels. It is possible that the systemic delivery of the genes of anti-inflammatory serpins (AAT and elafin) may also result in protective effects in islet transplantation.

### 5.1.18 Experimental Design

To test the systemic effects of AAT and elafin on protecting islet transplantation, untransduced islets from various donor strains are transplanted to diabetic recipients, which are intramuscularly injected with rAAV1-CB-AAT or rAAV1-CB-Elafin (Table 2). The donor/recipient pairs in these studies are outlined in Table 1. Islet isolation, islet transplantation and graft survival analysis follow the procedures described previously hereinabove.

TABLE 2

| | Vectors | Dose (particles) | N = | |
|---|---|---|---|---|
| 1 | RAAV-CB- | 1 × 10¹¹ | 10 | Monitoring blood glucose |
| | mAAT | | | (weekly), hAAT, elafin |
| | | | | and IAA (every 4 weeks) |
| | | | | levels. |
| 2 | RAAV-CB- | 1 × 10¹¹ | 10 | |
| | Elafin | | | |
| 3 | Control-vector | 1 × 10¹¹ | 10 | |
| 4 | Saline | | 10 | |

### 5.1.19 Vector Administration

Diabetic female NOD mice are injected intramuscularly into the caudal muscle of the pelvic limbs. The total injection volume is 100 μl. Since transgene expression from skeletal muscle takes 4 weeks to reach 50% of the peek levels (at 7 weeks), islet cell transplantation is performed 3 weeks after vector injection. To keep the animal alive, an insulin pellet (sustained-release bovine insulin 0.1 unit/day/pellet) is implanted under dorsal skin, at the time of vector injection. This pellet is then removed at the time of transplantation. For nude and NOD male recipient mice, induction of diabetes by streptozotocin is performed 4 weeks after rAAV vector injection. The islet transplantation then follows after hypoglycemia occurs.

### 5.1.20 Detection of Transgene Expression

Blood samples (50 ml/mouse) are collected via tail vein under sedation of isoflurane inhalation at 8 weeks after injection and at end of the experiment or the time that animals are sacrificed due to diabetes. Serum levels of hAAT and elafin in the mice transduced with rAAV vectors, or PBS are detected by ELISA and Western Blot, respectively.

ELISA for detecting hAAT is performed routinely (Song et al., 1998; Song et al., 2001a; Song et al., 2001b). Briefly, microtiter plates (Immoulon 4, Dynex Technologies, Chantilly, Va.) are coated with 100 μl of goat anti-hAAT (1:200 diluted, Sigma Immunochemical, St. Louis, Mo.) in Voller's buffer overnight at 4° C. Duplicated standard curves (hAAT, Sigma Immunochemical, St. Louis, Mich., USA) and serially diluted unknown samples are incubated in the plate at 37° C. for 1 hr. After blocking with 3% bovine serum albumin (BSA), a second antibody, rabbit anti-hAAT (1:1000 diluted, Roche Molecular Biochemicals, Indianapolis, Ind., USA) is reacted with the captured antigen at 37° C. for 1 hr. A third antibody, goat anti-rabbit IgG conjugated with peroxidase (1:800 diluted, Roche Molecular Biochemicals, Indianapolis, Ind.) is incubated at 37° C. for 1 hr. The plate is washed with PBS-Tween 20® between reactions. After reaction with the substrate (o-phenylenediamine, Sigma Immunochemical, St. Louis, Mo.) plates were read at 490 nm on a MRX microplate reader (Dynex Technologies). It is notable that no or very little cross-reaction to murine AAT has been observed using this ELISA. Serum levels of elafin are then detected by Western Blot using a monoclonal antibody against FLAG epitope tag (Zymed, Laboratory, San Francisco, Calif.).

Animals are anesthetized and sacrificed by cervical dislocation. The muscle at the injection site is harvested immediately and placed in appropriate fixatives. The following tissues are then collected for routine histopathological analysis: liver, heart, lungs, kidney, small intestine, pancreas, spleen, brain and gonad.

For detections of hAAT or FLAG-tagged elafin, all tissues from each animal are fixed in 4% paraformaldehyde, embedded in paraffin, or frozen in OCT, and sectioned. Tissue sections are then deparaffinized and rehydrated with water. Following a peroxide blocking step, tissue is then blocked with goat serum (Kirkegaard & Perry Laboratories, Gaithersburg, Md.). Tissue sections are incubated in antibody solution at 37° C. for 20 min. For hAAT, rabbit anti hAAT (Roche Molecular Biochemicals) is diluted to 1:100. For elafin, antibody against the FLAG tag (Zymed) is diluted to 1:200. Detection will be carried out using the True Blue™ peroxidase kit (Kirkegaard & Perry Laboratories). Tissue is counterstained with orcein (Kirkegaard & Perry Laboratories) prior to dehydration. All sections are permanently mounted with permount. Semiquantitative assessment of the percentage of parenchymal involvement is based on rigorous review of the randomly selected fields as previously described (Brass et al., 1993).

### 5.1.21 Vertebrate Animals

Freshly isolated mouse islets from different strains (100 islet/mouse) are transduced (400 per animal, 1×10⁷ particle/islet equivalent) with rAAV vectors. The transduced islets are transplanted to groups of diabetic mice (n=10). The rAAV1-mutant AAT and PBS serve as controls. The selected donor/recipient pairs are listed in Table 1. Graft survival is calculated as the number of days before diabetes recurrence.

The following number of animals were typically used for the study: C57BL/6 Donors: 360=40 mice for 10 recipients×9 groups of recipients; Female NOD recipients: 160=10 mice per group×16 groups; Male NOD donors: 160=40×4; male NOD recipients: 40=10×4. Total male NOD=200; and Nude mice: 40 recipients in control group.

In this study, untransduced islets from various donor strains are transplanted to diabetic recipients, which are intramuscularly injected with rAAV1-CB-AAT or rAAV1-CB-Elafin, or both vectors (Table 2). The donor/recipient pairs in the study are the same as outlined in Table 1.

C57BL/6 mice are purchased from the Mouse Colony Core (University of Florida, Gainesville, Fla.); female, male NOD and nude mice from Jackson Labs (Bar Harbor, Me.).

NOD male and nude recipients are rendered diabetic by a single intraperitoneal injection of 220 mg/kg streptozotocin (STZ, Sigma, St Louis, Mo.) freshly dissolved in citrate buffer. The mouse is grasped, and held in dorsal recumbency in a head-down position. The injection is made in the lateral aspect of the left lower quadrant. A short bevel, 0.5 in., 27 G needle is inserted through the skin and musculature and immediately lifted against the abdominal wall, which will aid in avoiding puncture of the abdominal viscera. Immobilizing the left leg is also essential in reducing this risk. STZ treated animals will develop diabetes within 48 hr. On these animals, blood glucose levels are checked daily post-STZ injection and once a week after transplantation.

Diabetes occurrence is defined as two consecutive non-fasting blood glucose levels of above 250 mg/dl. For spontaneous diabetes models, the development of Type I diabetes is highly variable; with onsets from 13 to 30 weeks commonplace. About 50% of female NOD mice from Jackson Labs develop diabetes by 15 weeks of age. The female NOD mice are ordered at 10 weeks of age so this would amount to an expected average of 5 weeks of pre-transplant glucose monitoring. Once a week monitoring is sufficient for both the pre- and post-transplant period.

In the allograft models (female and male NOD recipients), the rejection and recurrence of diabetes takes place usually within 2 weeks, so only 2 to 3 measurements are necessary. The expected survival of genetically modified islets is probably less than 2 months (based on data from similar studies in other organ systems), which amounts to 8 to 10 weeks of monitoring. The islet transplant survival in the double gene transfer group is not known, however the literature suggests 100 days as a “long-term survival” and the study will be terminated at this point which equals 14 weeks of post-transplant glucose monitoring (14 blood samples).

Blood glucose on whole blood samples is collected from the tail vein using a strip glucometer (Elite, Bayer). The blood samples are taken once a week from the tail vein by initial tail clipping followed by scab removal or needle puncture for the subsequent collections. A blood droplet is collected with a heparinized capillary tube. The tail is then cauterized with silver nitrate sticks to seal the wound. For this procedure, the mice are immobilized in a commercial plastic restraining cage. Blood glucose measurements are also performed once a week after islet transplantation. For the purpose of islet isolation, mice are euthanized by means of cervical displacement following a general anesthesia in a carbon dioxide chamber. For islet transplantation, the general anesthesia is induced. Under aseptic conditions, a right lumbar incision is performed and the right kidney exposed. A small incision is made in the kidney capsule in the superior pole area through which polyethylene tubing (PE-50) is gently introduced into the subcapsular space and advanced towards the opposite pole of the kidney. At that time, islets are slowly injected using an attached micrometric Hamilton syringe and allowed to spread at the pole. The catheter is then removed and the capsule opening cauterized. The kidney is then repositioned, muscle sutured, and skin stapled. After the surgery, animals are kept on a heating pad and monitored until they have recovered. The animals are observed daily thereafter. Animals are closely monitored for symptoms of pain such as guarding the painful area, vocalizing, licking, biting, self-mutilation, restlessness, lack of mobility, failure to groom, abnormal posture, failure to show normal patterns of inquisitiveness and failure to eat or drink. For the post-surgical pain management, buprenorphine is used at a dose of 0.1-0.5 mg/kg subcutaneously. Should they show any signs of wound dehiscence or wound infection (swelling, redness), the animals are removed from the group and euthanized. The skin staples are removed 10-12 days post surgery. The other transplants recipients are euthanized at the point of diabetes recurrence. In this case, also the graft-bearing kidney is removed for histological studies.

### 5.2 Example 2

### rAAV Mediated IL-10 Gene Delivery Prevents Type I Diabetes in NOD Mice

The development of spontaneous autoimmune diabetes in nonobese diabetic (NOD) mice provides for their use as a model of human Type I diabetes. To test the feasibility of muscle-directed gene therapy to prevent Type I diabetes, recombinant adeno-associated virus (rAAV) vectors containing murine cDNAs for immunomodulatory cytokines IL-4 or IL-10 were developed. Skeletal muscle transduction of female NOD mice with IL-10, but not IL-4, completely abrogated diabetes. rAAV-IL-10 transduction attenuated the production of insulin autoantibodies, quantitatively reduced pancreatic insulitis, maintained islet insulin content, and altered splenocyte cytokine responses to mitogenic stimulation. The beneficial effects were host specific, as adoptive transfer of splenocytes from rAAV-IL-10-treated animals rapidly imparted diabetes in naive hosts, and the cells contained no protective immunomodulatory capacity, as defined through adoptive cotransfer analyses. These results indicate the utility for rAAV, a vector with advantages for therapeutic gene delivery, to transfer immunoregulatory cytokines capable of preventing Type I diabetes. In addition, these studies provide foundational support for the concept of using immunoregulatory agents delivered by rAAV to modulate a variety of disorders associated with deleterious immune responses, including allergic reactions, transplantation rejection, immunodeficiencies and autoimmune disorders.

The etiology of Type I diabetes in NOD mice is both complex and multifactorial (Bach, 1994; Atkinson and Leiter, 1999). Both CD4⁺ and CD8⁺ T cells comprise the effector arm, with underlying functional defects in bone marrow-derived antigen-presenting cells (macrophages, dendritic cells, B lymphocytes) shown to be essential components in the selection and activation of the autoimmune repertoire (Serreze, 1993; Wong and Janeway, 1999). The destruction of β cells apparently entails both necrotic and apoptotic events in response to invasion of the islets by leukocytes (Yoon et al., 1998; Trudeau et al., 2000). Autoreactive T cells are targeted against multiple autoantigens, including insulin and glutamic acid decarboxylase (Tian et al., 1998; Wegmann and Eisenbarth, 2000).

Previous studies indicate that the pathogenic facet of the β cell destructive immune response in nonobese diabetic (NOD) mice is biased toward T helper 1-like immunities (Wong and Janeway, 1999; Tian et al., 1998). Depending on the time and mode of administration (early vs. late, systemic vs. local), treatment with the immunoregulatory cytokines IL-4 or IL-10 can inhibit the development of Type I diabetes in NOD mice as well as prevent the recurrence of disease, either alloimmune and/or autoimmune, in mice receiving islet transplants (Rapoport et al., 1993; Wogensen et al., 1994; Pennline et al., 1994; Rabinovitch et al., 1995; Cameron et al., 2000). However, given their relatively short half-lives, the practicality of using these cytokines for initiation of immune deviation would be currently limited because of the need for repeated administration. It has been demonstrated that sustained and stable production of secreted proteins can be achieved in vivo through recombinant adeno-associated virus (rAAV)-mediated gene delivery into skeletal muscle (Kessler et al., 1996; Song et al., 1998). rAAV vectors have become increasingly recognized as having some superiority to other viral and nonviral gene delivery systems with regard to their safety, efficiency, lack of need for repeated viral administration, duration of action without known pathology, and only the occasional induction of modest immune responses (Muzyczka, 1992; Rabinowitz and Samulski, 1998). A cellular modification toward the in vivo production of cytokines, achievable by rAAV gene transfer, could be exploited for developing novel intervention protocols for Type I diabetes and other immune system-based disorders (Kapturczak et al., 2001).

### 5.2.1 Materials and Methods

5.2.1.1 Plasmid Construction, Viral Packaging, and Production, Cellular Transfection, and Transduction

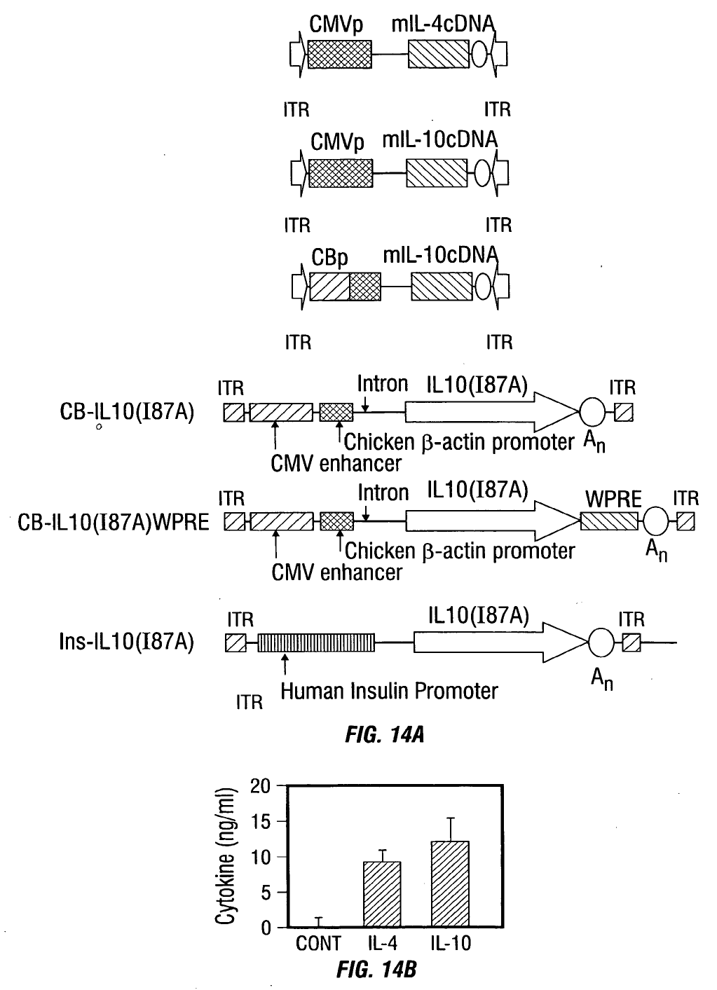

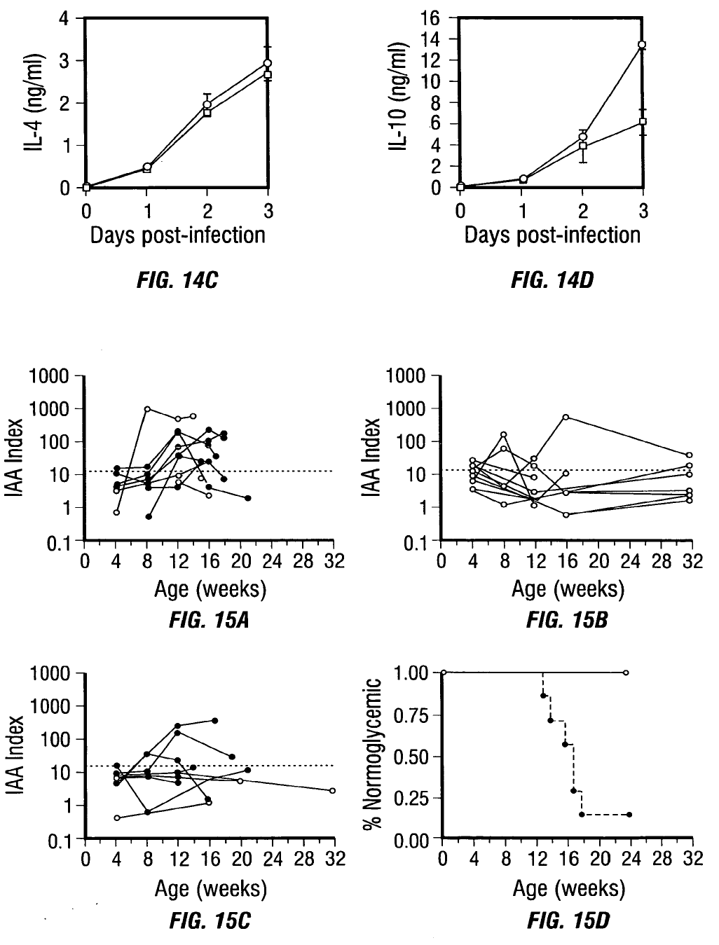

Various illustrative rAAV vector constructs are depicted diagrammatically in FIG. 14A. Because cellular IL-10 (cIL-10) has been shown to act as a bifunctional molecule, it posses strong immunosuppressive activity demonstrated by its ability to turn off cytokine production by T cells (Fiorentino et al., 1989). It also posses immunostimulatory activity under certain circumstances (Fei et al., 1990, Thompson-Snipes et al., 1991, Rousset et al., 1992). It can act as a stimulatory factor for immature and mature thymocytes, mast cells, and B cells. Ding et al., (2000) identified a single amino acid at position 87 of murine IL-10 as important for the immunostimulatory activity. By replacing an isoleucine with an alanine at position 87 Ding et al were able to abrogate the immunostimulatory activity of cIL-10.

To exploit this modification, several rAAV expression vectors have also been created that express this altered form of cIL-10 that contains the isoleucine to alanine change at position 87 [cIL-10(I87A)]. The cIL-10(I87A) has been placed behind the CMV enhancer/and under the control of the chicken β-actin promoter (CB). A woodchuck hepatitis virus post-transcriptional regulatory element was also added to help stabilize the RNA and hence increase protein expression (FIG. 14A). This constructs provides for a high level of protein expression in a wide variety of cell types and tissues. The cIL-10(I87A) gene has also been placed under the control of the human insulin promoter for high level regulated expression in pancreatic islet cells. The rAAV-CB-cIL-10(I87A) vector has been exploited for in vivo use in the NOD-scid mouse model of Type I diabetes to demonstrate the efficacy of these constructs in mammalian systems.

Murine cDNAs for the cytokines IL-4 and IL-10 were cloned into the p43.2 plasmid. rAAV2 production, titer determination, and infectivity were performed as described (Hauswirth et al., 2000). Transfection (5 μg DNA, Superfect; Qiagen, Chatsworth, Calif.) and transduction of myoblast C2C12 cells were performed as described (Song et al., 1998). For studies using adenovirus as a helper virus, myoblasts were treated with adenovirus type 5 at a multiplicity of infection of 5 for 2 hr (37° C., 5% CO₂) before coinfection with rAAV.

### 5.2.1.2 Mice

Specific pathogen-free NOD.MarTac mice (Taconic Farms) were housed in a BSL-2 barrier facility. Blood glucose levels were determined weekly/biweekly, with animals considered Type I diabetic when levels exceeded 240 mg/dl on two consecutive occasions, greater than 24 hr apart.

5.2.1.3 rAAV Vector Administration

Four-week-old female NOD mice were injected intramuscularly into the caudal muscle of the pelvic limb. These injections used 100 μl of saline, saline containing 1×10¹⁰ units of either rAAV-IL4 or rAAV-IL-10, or saline containing the latter two in combination (1×10¹⁰ units of rAAV-IL4 and 1×10¹⁰ units of rAAV-IL-10) per mouse.

### 5.2.1.4 Cytokine, Serum IgE, and Insulin Autoantibody Analysis

Supernatant cytokine levels for IL-2, -4, -10, and IFN-γ as well as serum IgE were measured with the use of OPTEIA kits (PharMingen) (She et al., 1999), with serum IL-10 assessed by microbead cytokine assay (Upstate Biotechnology, Lake Placid, N.Y.). Insulin autoantibodies (IAAs) were measured by RIA with radiolabeled insulin (Amersham Pharmacia) and protein A Sepharose (Sigma) (She et al., 1999). An index was calculated as [(unknown cpm−negative control cpm)/(positive control cpm−negative control cpm)]×100. The cutoff of 12.2 was chosen on the basis of the mean index+3 SD of 30C57/BL6 mice.

### 5.2.1.5 Histological Analysis

Skeletal muscle samples were paraformaldehyde (4%) fixed, paraffin embedded, and hematoxylin/eosin stained. Insulitis was evaluated on hematoxylin/eosin-stained frozen sections of pancreas and scored on a blind basis with a standardized scoring system described by others (Arreaza et al., 1997). Pancreata were also stained for insulin with the use of antiporcine insulin (Dako) and intercellular adhesion molecule-1 (PharMingen) on frozen and paraffin sections, respectively.

### 5.2.1.6 Reverse Transcription-PCR™ for Detecting Transgene Expression

Total RNA from the injection site or cells transduced with rAAV vector was purified and treated with RNase-free DNase (RNAqueous-4PCR™; Ambion, Austin, Tex.). First-strand cDNA synthesis was performed with Maloney murine leukemia virus reverse transcriptase and random cecamer primers (RETROscript; Ambion). The cDNA was amplified by nested PCR™. For detection of transcript from rAAV-IL4, the first PCR™ reaction was performed with primers, P1, 5′-CAGTCTCGAACTTAAGCTGC-3′ (SEQ ID NO:52), and P2, 5′-GGACTTGGACTCATTCATGG-3′ (SEQ ID NO:53), for 35 cycles. Two percent of the reaction was used for the second PCR™ with primers, P3, 5′-CAGAAGTTGGTCGTGAGGCA-3′ (SEQ ID NO:54), and P4,5′-GCAGCTCCATGAGAACACTA-3′ (SEQ ID NO:55), for 35 cycles. The final PCR™ product was cloned into a TA-cloning vector and sequenced to confirm that the transcript was from rAAV cytomegalovirus (CMV)-IL-4.

### 5.2.1.7 Splenocyte Studies

Splenocytes were cultured at 5×105 cells per well in 200 μl of RPMI 1640 medium (10% FBS) in 96-well round-bottom plates. Supernatants were collected at 24 and 48 hr for cytokine analysis in response to Con A. For studies of in vivo activity, 8-week-old male NOD mice were irradiated (700 rads) and injected via the tail vein with splenic lymphocytes (2×10⁷) obtained from 20-week-old newly diagnosed diabetic NOD mice or 32-week-old rAAV-IL-10-treated NOD mice under conditions of either adoptive transfer or at a 1:1 combination (adoptive cotransfer) (Bowman et al., 1996).

### 5.2.1.8 Statistical Analysis

Data are presented as the mean±SEM. Student's t tests and ANOVA testing were used for analyses comparing the different groups, with statistical significance considered if P<0.05.

### 5.2.2 Results

5.2.2.1 Effect of rAAV-Delivered Immunomodulatory Cytokines on Type I Diabetes

To validate function, mouse myoblasts were either transfected with plasmids or transduced with packaged rAAV virions expressing L-4 and -10 (FIG. 14A). Specifically, C2C12 myoblast cells were transfected with CMV-IL-4 or CMV-IL-10 plasmids or virally transduced with rAAV-CMV-IL-4 or rAAVCB-IL-10. The transduction studies were performed in the presence and absence of adenovirus, a helper virus that aids in the conversion of rAAV from single-stranded to double-stranded DNA (Muzyczka, 1992; Rabinowitz and Samulski, 1998). At 48 hr, plasmid-transfected cells readily expressed either IL-4 or -10 (FIG. 14B), whereas control cells transfected with control green fluorescent protein failed to produce these cytokines. Similarly, within 24 hr, production of L-4 and -10 was observed in supernatants from rAAV-CMVIL-4- and rAAV-CB-IL-10-transduced cells (FIG. 14C and FIG. 14D) and did not depend on coinfection with adenovirus.

To observe the effects of skeletal muscle production of these cytokines on the development of Type I diabetes in vivo, female NOD mice at 4 weeks of age were intramuscularly injected with purified vector preparations of rAAV-IL-4 and/or rAAVIL-10. rAAV-IL-10 transduction completely abrogated the development of diabetes (0/10; 0% incidence at 30 weeks). Additionally, mice receiving the combination therapy of both rAAV-IL-10 and rAAV-IL-4 were also protected from the disease (0/10; 0%). This protection was associated with rAAVIL-10, only as rAAV-IL-4-treated animals did not display a significant delay in the kinetics of disease development nor did they demonstrate long-term differences in disease frequency (8/10; 80%) when compared with control animals (7/10; 70%).

5.2.2.2 Confirmation of Functional rAAV Transduction of Skeletal Muscle

To assess transgene function, serum cytokine levels were determined in a separate series of similarly treated animals injected with rAAV vectors and killed at 10-12 weeks of age; studies identified elevated IL-10 levels in rAAV-IL-10-transduced animals (67.5±14.9 pg/ml; n=4) that were not detectable in saline controls (below assay detection limits of 15.6 pg/ml; n=4). In contrast, serum IL-4 levels were not elevated/undetectable in rAAV-IL-4-transduced animals in comparison with controls. However, as an indirect indicator of biological activity in transduced animals, total serum IgE was elevated in rAAV-IL4-treated animals, consistent with the known actions of IL-4 on IgE production and the difficulty of measuring serum cytokines (Fellowes et al., 2000; Chang and Prud'homme, 1999; Shelburne and Ryan, 2001). The site of injection was examined to observe the local effects of transgene expression. Whereas normal muscle histology was observed in saline-injected animals, the introduction of rAAV-IL-10 into muscle induced a mild degree of lymphocytic accumulation, and rAAV-IL-4 induced a mild to severe degree of lymphocytic accumulation, observations consistent with the action of these cytokines on immunological recruitment and proliferation (Rabinovitch, 1998). Furthermore, this lymphocytic accumulation appeared transgene-specific, as injections of NOD mice with rAAV-α-1-antitrypsin failed to induce abnormal muscle pathology and was similar to that of saline controls, a finding consistent with the “nonimmunogenic” property often ascribed to rAAV. Additional evidence of skeletal muscle transduction was obtained by reverse transcription-PCR™ analysis of muscle, taken from injection sites of 16- to 30-week-old animals, with the use of cytokine-specific primers and subsequent sequencing of products. These studies confirmed the specific presence of IL-4 in rAAV-IL-4-injected and IL-10 in rAAV IL-10-treated animals as well as the lack of these two cytokine genes in saline-treated animals. Furthermore, transgene retention was suggested by the presence of reverse transcription-PCR™ products in animals at 30 weeks of age. Longitudinal analysis of control, rAAV-IL-4-transduced, and rAAV-IL-10-transduced animals in the period before the onset of diabetes would be expected (4-12 weeks) revealed no differences in blood glucose values, suggesting that the systemic introduction of rAAV-expressed transgenes also did not interfere with β cell function.

5.2.2.3 Mechanisms by which rAAV-IL-10 Confers Protection

A key feature of Type I diabetes in NOD mice is the infiltration of the endocrine pancreas with inflammatory cells. In contrast to a normal islet (stage 0 insulitis), the mildest form of inflammation is infiltration with inflammatory cells (dendritic cells, macrophages, T and B cells) around the perivascular duct and peri-islet regions of the islets of Langerhans (“peri-insulitis,” stage 1 insulitis). This peri-insulitis process in NOD mice normally begins when the animals are 4-6 weeks of age and is followed by an increase in the number of affected islets, a progressive increase in the quantity of intra-islet inflammatory cell accumulation (stages 2 and 3), and the selective destruction of insulin-producing islet β cells (loss of insulin content). To examine the immunomodulatory effect of rAAV-cytokine gene therapy on the insulitis lesion before the period of developing overt clinical disease, insulitis was monitored in a separate series of animals injected with rAAV vectors and killed at 10-14 weeks of age. Cytokine transduction of NOD mice with rAAV-IL-10 and, to a lesser extent, with rAAV-IL-4 effectively reduced the quantitative parameters of insulitis in recipient animals. Specifically, pancreatic sections from all mice contained islets free of inflammation as well as islets that demonstrated moderate to severe insulitis. However, the percentage of islets affected by severe insulitis was far less in the pancreata from rAAV-IL-10 NOD mice than in control animals, with rAAV-IL-4-treated mice forming an intermediate group. Furthermore, insulin content appeared to be retained and at higher levels in islets of rAAV-IL-10-transduced mice compared with those from the rAAV-IL-4 or control group mice. Finally, previous studies have suggested that the expression of intercellular adhesion molecule-1 in islets, as influenced by the systemic or localized production of IL-10, is associated with diabetogenesis (Balasa et al., 2000a). However, studies analyzing four pancreatic structural components representing extra- and intra-islet vasculature did not reveal substantial differences between intercellular adhesion molecule-1 staining among animals from the three subject groups. These findings indicate that rAAV-IL-10 gene therapy in part inhibits diabetes by reducing the severity of insulitis.

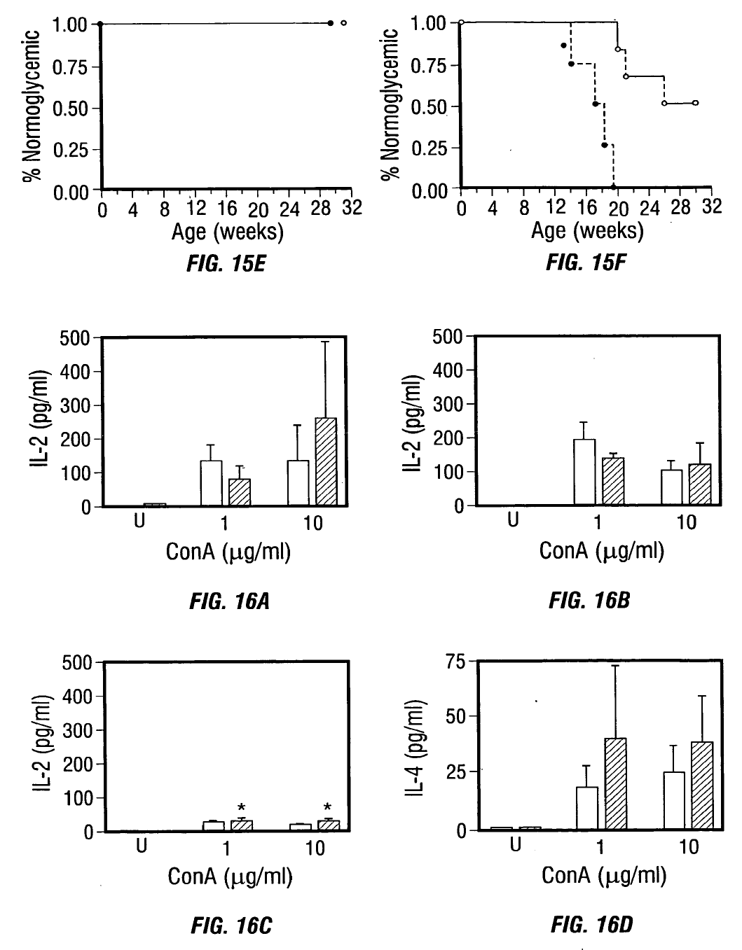

A recent report suggested that IAAs provide an excellent predictor of future development of Type I diabetes in NOD mice, inasmuch as a majority of animals developing disease possessed this marker by 12 weeks of age (Yu et al., 2000). Hence, longitudinal analysis of IAA was performed in rAAV-IL-10, rAAV-IL-4, and control animals. As expected, serums from a minority of animals at 4 weeks of age were IAA positive (FIG. 15A, FIG. 15B and FIG. 15C). In saline controls, both longitudinal (FIG. 15A) and life table analysis (FIG. 15D) of individual animals suggested a strong association between IAA development and formation of Type I diabetes. Indeed, all saline-treated animals developing Type I diabetes developed IAA by 16 weeks of age. Interestingly, the effect of rAAV-IL-10 treatment appeared to involve a reduction in IAA index (FIG. 15B) in the period beyond 8 weeks of age in nearly all animals. The observed effect of rAAV-IL-4 was less clear, with no specific pattern associated with protection from disease (FIG. 15C and FIG. 15F). Hence, it is possible that the protection from Type I diabetes observed in rAAV-IL-10-treated mice resulted to some degree from the attenuation of islet autoantigen specific immunity in vivo.

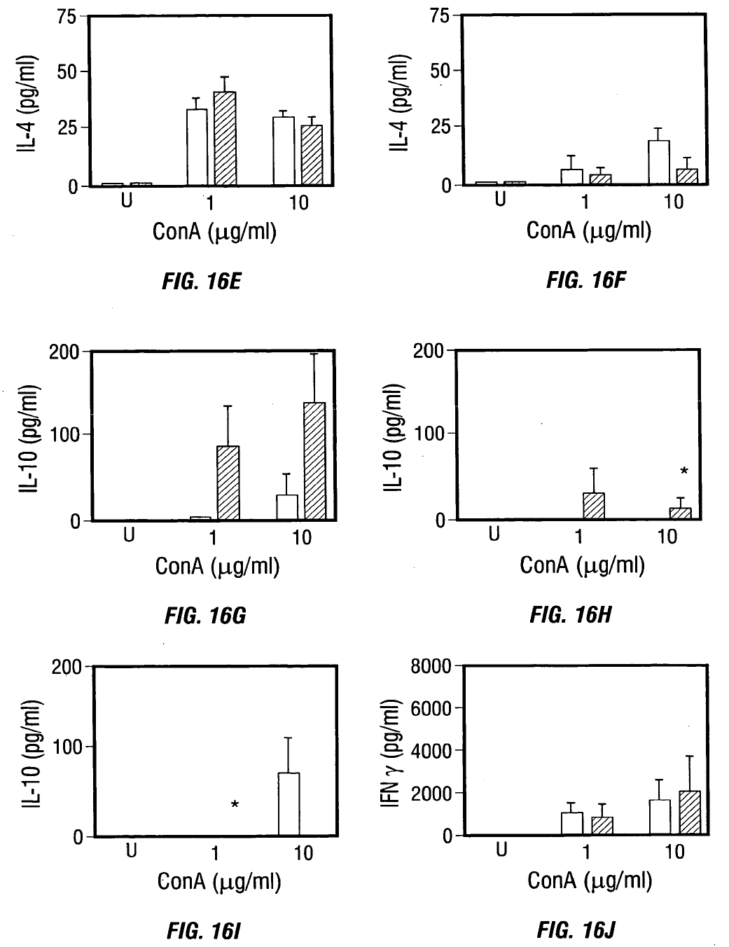

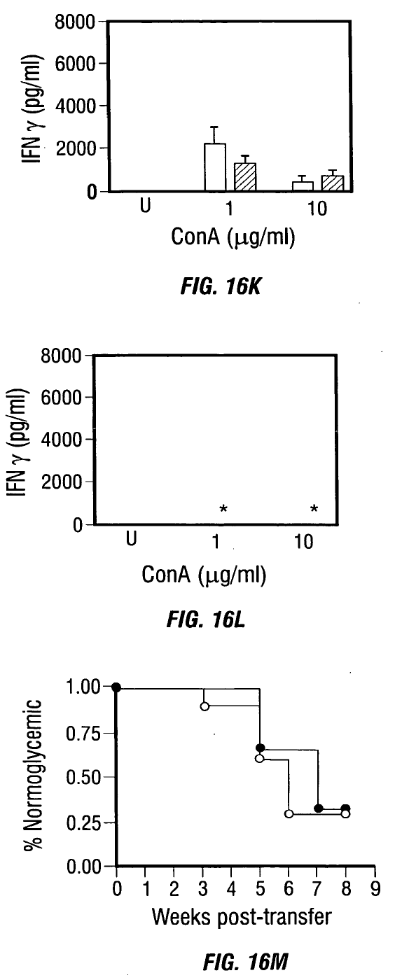

To learn whether protection from Type I diabetes afforded by rAAV-IL-10 could have resulted, in part, from the induction of a shift in systemic cytokine production, the levels of IL-2, -4, -10, and IFN-γ produced by splenocytes were analyzed in response to mitogenic stimulation with Con A. In comparison with saline-treated animals (FIG. 16A, FIG. 16D, FIG. 16G and FIG. 16J), rAAV-IL-4-transduced mice (FIG. 16B, FIG. 16E, FIG. 16H and FIG. 16K) produced equivalent levels of IL-2, -4, and IFN-γ, whereas IL-10 production was markedly diminished. Quite strikingly, the introduction of rAAV-IL-10 into skeletal muscle resulted in markedly diminished production of IL-2, -4, -10, and IFN-γ (FIG. 16C, FIG. 16F, FIG. 16I and FIG. 16L). These results suggest that of the cytokines tested, a reduced production of splenocyte-derived IL-2 and IFN-γ may have been more closely associated with protection than IL-10 and possibly IL4, as only the rAAV-IL-10-treated animals displayed diminished disease.

Finally, to learn whether rAAV-IL-10 transduction modulates Type I diabetes by altering the β cell destructive capacity and/or inducing immunoregulatory cells in vivo, both adoptive transfer and adoptive cotransfer studies were performed. Specifically, young (nondiabetic) irradiated male NOD mice were injected via the tail vein with splenocytes from either rAAV-IL-10-treated mice or from newly diabetic NOD mice. In addition, a third group of recipients was injected with a 1:1 mixture of splenocytes from rAAV-IL-10-treated animals and from newly diabetic NOD mice. Interestingly, Type I diabetes developed in 50% of the rAAV-IL-10 transferred animals by 4 weeks post-transfer, in a time frame similar to that of newly diagnosed animals (FIG. 16M). Furthermore, recipient mice injected with equal mixtures of splenocytes from the rAAV-IL-10-protected animals and newly diagnosed NOD mice developed diabetes in an accelerated time frame (50% by 3 weeks after transfer), whereas control time frame (50% by 3 weeks after transfer), whereas control irradiated males not subject to splenocyte transfer failed to develop diabetes within 8 weeks after transfer. These studies suggest that rAAV-IL-10 transduction did not induce immunoregulatory cells in vivo and that the mechanism of prevention is host specific. This conclusion further implies that the beneficial effects require the continuous expression of the IL-10 transgene, an important feature of rAAV vectors.

### 5.2.3 Discussion

These studies demonstrate the utility of rAAV-mediated gene delivery, specifically that involving IL-10, as a method of preventing Type I diabetes. In addition to primary disease prevention, the ability of rAAV to transduce islet cells has been demonstrated (Prasad et al., 2000; Flotte et al., 2001). The delivery to islets of anti-inflammatory cytokines, cytoprotective antioxidant, and anti-inflammatory enzymes, and/or anti-apoptotic molecules by rAAV delay/prevent the recurrence of Type I diabetes in islet transplantation and offer a new form of immunotherapy for this disease.

Previous studies of IL-10 in NOD mice have been described as “paradoxical” (Balaji and Sarvetnick, 1996). Transgenic BALB/c mice expressing IL-10 in the pancreas exhibited peri-insulitis but not insulitis or diabetes (Wogensen et al., 1993). However, backcrossing of these transgenic mice onto the NOD background, rather than leading to protection, leads to disease acceleration, suggesting a potential pathogenic role for IL-10 in Type I diabetes development (Moritani et al., 1994). In contrast, administration of IL-10 to adult NOD mice attenuated Type I diabetes, a finding consistent with disease prevention (Nitta et al., 1998). One potential means for this variance may be the contrasting effects of local (islet) vs. systemic production (Balasa et al., 2000b).

### 5.3 Example 3

### rAAV Transfection of Islet Cells Results in Therapeutic Levels of Interleukin Expression

Islet transplantation can be used to treat Type I diabetes, yet persisting alloimmune and autoimmune responses represent major obstacles to clinical success for this procedure. Studies from animal models suggest in a delivery specific-fashion (systemic administration and/or local cellular expression), anti-inflammatory cytokines, e.g., interleukin-4 (IL-4), IL-10, can delay/prevent recurrent Type I diabetes in islet transplantation. Hence, the selective administration of immunosuppressive cytokines to islet cells or skeletal muscle by targeted gene delivery would appear to offer a promising form of immunotherapy. However, most viral gene delivery systems (e.g., adenovirus) utilized to date have demonstrated significant limitations in practicality due to the level and duration of recombinant transgene expression as well as their induction of host immunogenicity to vector proteins. A series of recombinant adeno-associated virus (rAAV) vectors have been developed which, when transfected into islet cells or mouse myoblasts, demonstrate stable high-level expression of recombinant cytokine transgenes. The objective is to establish a method affording the prevention of Type I diabetes in cases of islet cell transplantation for the reversal of the disease. The hypothesis that Type I diabetes can be prevented in NOD mice through the use of rAAV based gene transfer of IL-4 and IL-10 will be tested. The experiments determine the effect(s) of local and systemic cytokine transgene expression on anti-islet cell immunity, islet cell metabolism and therapeutic efficacy in combination with rAAV delivered anti-oxidant transgenes (manganese superoxide dismutase, heme oxygenase-1). In addition to testing a novel model for reversing Type I diabetes, these studies will provide information vital to understanding the immunoregulatory mechanisms critical to the development of both alloimmune and autoimmune islet cell rejection mechanisms and recurrent Type I diabetes.

### 5.3.1 Islet Cell Transplantation

The physiological replacement of insulin producing cells afforded by islet cell transplantation represents an exciting alternative to exogenous insulin administration as, when technically successful; it offers excellent metabolic control (Hering et al., 1993; Kenyon et al., 1996; Rosenberg, 1998; Cretin et al., 1998). Previous clinical trials have established that long-term, i.e., more than 7 years, function of transplanted islets can be observed in selected recipients (Alejandro et al., 1997; Warnock et al., 1991; Scharp et al., 1991; Socci et al., 1991). However, for a vast majority of individuals, islet transplantation remains unsuccessful, with a substantial percentage of recipients losing graft function a short time after transplant. Among the likely reasons for this relative lack of clinical success is the action of several concurrent mechanisms including rejection, recurrence of anti-islet cell autoimmunity, and non-specific islet loss immediately after transplantation due to perturbation of the graft microenvironment (inflammation, ischemia/reperfusion) (Kaufman et al. 1990, Weir et al., 1990; Stevens et al., 1994; Nussler et al., 1992; Bottino et al., 1998). For islet transplantation to become a clinical reality, a need exists to devise strategies of immunosuppression/immunomodulation that are substantially different from those presently utilized. The reasons for this need include: the apparent inadequacy of currently available immunosuppressive agents to reproducibly promote long-term islet graft survival; the direct toxic effects of these drugs on islet function; and serious unwanted side effects linked to chronic immunosuppression (Hering et al., 1993; Kenyon et al., 1996; Rosenberg, 1998; Cretin et al., 1998; Massetti et al., 1997; Penn, 1989; Dunn, 1990; Jindal, 1994; Hahn et al., 1986; Hirano et al., 1992; Venkatesan et al., 1987; Guo et al., 1997). Indeed, conventional immunosuppressive agents routinely used in islet transplant patients, e.g., cyclosporine, FK506, and steroids, are characterized by intrinsic diabetogenic effects imposing a two- to three-fold increase in the metabolic demand of islet cells (Jindal, 1994; Hahn et al., 1986; Hirano et al., 1992; Venkatesan et al., 1987; Guo et al., 1997).

### 5.3.2 NOD Mice as a Model for Type I Diabetes

The NOD mouse provides an excellent model system to investigate disease pathology and intervention strategies to prevent human Type I diabetes (Atkinson and Maclaren, 1994). At three to five weeks of age, a mononuclear cell infiltrate of the pancreatic ducts and venules initiates with eventual progression to the pancreatic islets, i.e., insulitis. Whereas these early insulitis stages appear “non-destructive,” intra-islet invasion occurs at 12-16 weeks of age with this latter infiltrate associated with selective destruction of the insulin-secreting β cells (Fujita et al., 1982). The cellular infiltrate is heterogeneous, with a predominance of T cells followed by various percentages of macrophages, dendritic cells, and B-lymphocytes. Multiple lines of evidence suggest that both CD4+T-helper and CD8+T-cytotoxic lymphocytes play a role in the disorder (Bendelac et al., 1987; Miller et al., 1988; Wang et al., 1987; Like et al., 1986; Sibley and Sutherland, 1987; Haskins et al., 1988). Evidence for spontaneous β cell regeneration is limited, and allogeneic islets transplanted into diabetic recipients undergo a repeated episode of islet cell destruction.

### 5.3.3 Immunoregulaton of Cellular Immune Responses

While autoimmune β cell destruction in NOD mice appears mediated by T-cells (Bendelac et al., 1987; Miller et al., 1988), the development and activation of these effectors appears to be due in large part to an intrinsic inability to induce various immunotolerogenic functions (Oldstone, 1988; Shehadeh et al., 1994; Sandelain et al., 1990). In NOD mice, the autoimmune tissue destruction appears to be promoted when self-peptide reactive CD4⁺ T-cells produce a Th1 pattern of cytokines including IL-2 and γIFN which support macrophage activation, delayed type hypersensitivity responses, and immunoglobulin (Ig) isotype switching to IgG2a. In contrast, autoimmune tissue destruction appears to be blocked when self-peptide reactive CD4⁺ T-cells produce a Th2 pattern of cytokines (IL-4, IL-5, IL-6, IL-10, and IL-13) which provide help for the activation of B lymphocyte mediated humoral immunity and Ig isotype switching to IgG1 and IgE. Of the aforementioned cytokines, IL-4 appears to be most important in switching CD4⁺ T-cells from a Th1 to Th2 response profile. However, IL-10 also serves an important role by decreasing Th1, NK T cell, and macrophage functions as well as increasing B1 B-cell and macrophage activities. While a majority of studies on the Th1/Th2 model to date have focused on murine immune response and disease, an extensive body of literature supports (in part) the applicability of this model to humans (McAuthor and Raulet, 1993; Taylor-Robinson and Phillips, 1994; Parish et al., 1993).

### 5.3.4 Cytokine Therapy for Preventing Type I Diabetes and Disease Recurrence in Mice

A switch from Th2 to Th1 subsets appears to be a late event in pre-diabetes of NOD mice; converting the non-destructive lymphocytic infiltration of predominately Th2 activity into an aggressive destructive and pathogenic Th1 response (Liblau et al., 1995; Rabinovitch, 1994; Bach, 1995; Kroemer et al., 1996). Cytokines can be experimentally used to induce an immune deviation towards the Th2 phenotype and alter diabetes frequency; examples from a large body of literature show that systemic administration of IL-4 and IL-10 prevents disease in NOD mice (Rapoport et al., 1993; Pennline et al., 1994). These studies complement those indicating that detection of IL-4 in islets at the onset of inflammation identify non-destructive insulitis (Arreaza et al., 1997), and that NOD mice with pancreatic (insulin promoter) expression of IL-4 are protected from autoimmunity (Mueller et al., 1996). In addition, islet cell expression (transgenic) of IL-4 induces islet antigen specific Th2 cells that block the action of diabetogenic T cells in the pancreas (Gallichan et al., 1999), and may correct the aforementioned inherited defect in NOD mice of forming Th2 responses (Cameron et al., 1997). While “the picture” for Th2 immunity in spontaneous disease is promising yet unproven, the concept when applied to islet cell transplantation is far less clear. The dominant host response to an islet allograft does appear (through a variety of measures) as Th1, and diminished IFNγ/increased IL-4 and diminished IL-12/increased IL-10 are observed in long-term surviving grafts (Nickerson et al., 1994). However, a number of studies attempting to recreate the benefits of this pattern through targeted islet cell cytokine expression have failed to reveal effectiveness including IL-10 transgenic allografts (Wogensen et al., 1994), IL-4 & IL-10 (adenovirus) syngeneic grafts (Smith et al., 1997), and IL-4 transgenic allografts in vitro (Davies et al., 1999). In cases of xenogeneic transplantation, local IL-10 expression can even accelerate graft rejection (Deng et al., 1997). In contrast, other reports argue for therapeutic effectiveness including systemic-therapy with IL4 and IL-10 inhibiting diabetes recurrence in NOD mice transplanted with syngeneic islets (Rabinovitch et al., 1995), IL-4 transgenic islets resistant to disease when challenged with diabetogenic splenocytes (Mueller et al., 1996), and decreased alloreactivity in vitro to human islets secreting IL-10 (Benhamou et al., 1996). Perhaps most promising is the recent report of Gallichan et al. (1998) demonstrating that syngeneic islet grafts expressing lentiviral mediated IL-4 are protected from insulitis in an adoptive transfer model. While sometimes at conflict, this collective body would suggest that the site of therapeutic administration, cytokine action and concentration each play an important role in the success of therapeutic outcomes and may provide an explanation for the reportedly paradoxical effects. This concept finds support in other autoimmune models where local IL-10 administration reduces endotoxin-induced oscular inflammation whereas systemic delivery exacerbates disease pathology, and TGF-β provided locally induces arthritis yet systemic administration can attenuate inflammation (Balasa and Sarvetnick, 1996). Cytokine gene therapy strategies predominantly but not exclusively involve three modes of delivery (Schmidt-Wolf and Schmidt-Wolf, 1995; Robbins and Evans, 1996; Giannoukakis et al., 1999). Cells targeted for autoimmune attack may be genetically modified to express cytokines that protect them from immune-mediated destruction, i.e., target tissue gene therapy. Another strategy allows for autoreactive T cells to be genetically altered to deliver anti-inflammatory cytokines to autoimmune lesions, i.e., T cell mediated gene therapy. Finally, new advances in muscle delivery offer the hope of systemic cytokine production.

5.3.5 rAAV-Mediated Gene Therapy for Prevention of Diabetes

rAAV vectors are capable of stable in vivo expression (Flotte et al., 1993; Kaplitt et al., 1994; Xiao et al., 1996; Kessler et al., 1996; Fisher et al., 1997; Clark et al., 1997) with low immunogenicity (Jooss et al., 1998). AAV is a non-pathogenic human parvovirus whose life cycle includes a mechanism for long-term latency (Carter et al., 1990). In the case of wild-type AAV (wtAAV), this is due to site-specific integration on human chromosome 19 (AAVS1) (Kotin et al., 1992; Kotin et al., 1990) while with rAAV vectors, persistence occurs via a combination of episomal persistence and integration into non-chromosome 19 locations (Afione et al., 1996; Kearns et al., 1996; Ponnazhagan et al., 1997). rAAV latency also differs from that of wtAAV in that wtAAV is rapidly converted to double-stranded DNA in the absence of helper virus (e.g., adenovirus) infection, while rAAV leading strand synthesis is delayed in the absence of helper virus (Fisher et al., 1996; Ferrari et al., 1996). Recent evidence further supporting the concept that rAAV vector expression is robust and long-lived. Examples include the demonstrations that murine skeletal myofibers transduced by rAAV vector were capable of sustained secretion of human erythropoietin, apparently without eliciting an immune response against hEpo (Kessler et al., 1996); leptin in ob/ob mice (Murphy et al., 1997); and our own study demonstrating in vivo long-term, stable systemic-expression of α-1-antitrypsin (AAT) using rAAV-skeletal muscle transduction, with minimal immunogenicity (Song et al., 1998).

5.3.6 rAAV-Mediated Delivery of the Cytokine IL-4 into Murine Islet Cells

As previously described, investigations in NOD (or other recipient mice) have demonstrated that low doses, i.e., 50 ng/ml, of IL-4 protect against insulitis, spontaneous Type I diabetes, and recurrent disease in islet-transplanted recipients. This protocol involves continual systemic therapeutic administration at a rate of three times per week. While effective, the short half-life of IL-4 in vivo, i.e., approximately 20 minutes, provides a practical complication in terms of the need for repeated in vivo administration. IL4 production afforded by gene therapy could offer an improved alternative method by providing similar beneficial results in vivo through administration of vector-transgene into transplanted islet cells. In addition to the aforementioned evidence, this concept finds strong support through studies demonstrating therapeutic effectiveness utilizing lentiviral delivery of IL-4 into islets prior to transplantation (Fisher et al., 1996). We have demonstrated that rAAV can serve as a superior vector for delivery of such molecules in that high quantities, i.e., up to 800 μg/ml, of recombinant transgene can be produced for an extended period of time, i.e., >1 year in mice. Furthermore, we have established the ability of rAAV to both functionally transduce islet cells as well as impart IL-4 expression from these cells.

### 5.3.7 Experimental Methods

These in vivo studies investigate whether constitutive local (islet) IL-4 production imparts protection from recurrent Type I diabetes. rAAV-IL4 transduced islets (400 per animal) will be provided to groups of mice (n=8/group) as outlined in Table 3. Control animals will receive rAAV-AAT transduced islets. AAT will provide a control transgene with no extraordinary immune altering capacity. In terms of our selection of donor/recipient pairs, strep-treated Nude mice will be transplanted as a monitor of islet cell function in immunological absence. For studies of immunological rejection, C57BL/6 mice are H-2 incompatible with NOD and demonstrate prompt rejection of C57BL/6 islets transplanted in both diabetic (female) and non-diabetic (male) untreated NOD mice. NOD will be used as recipients of syngeneic (NOD) or allogeneic (C57BL/6) islets. Such a design is necessary to address the question of whether protection afforded by transgene expression is sufficient in situations of syngeneic versus allogencic transplants as well as in autoimmune (i.e., NOD) vs. strep-induced mice.

TABLE 3

| | Donor to | | | |
|---|---|---|---|---|
| | Recip | Donor to Recip | Donor to Recip | Donor to Recip |
| I. Function | C57BL/6 into | | | |
| transplant type | Nude (strep) | | | |
| | Allogeneic | | | |
| | Group A | Group B | Group C | Group D |
| II.Protection | C57BL/6 into | NOD (male) into | C57BL/6 into | C57Bl/6 into |
| Transplant type | NOD (femal) | (NOD) female | NOD | BalbC (strep) |
| | | | (male/strep) | |
| | Allo/Autoimm. | Syng/Autoimm. | Allogeneic | Allogeneic |

Because of the temporal aspects of rAAV transgene expression (i.e., approximately 3 weeks are necessary for transgene expression), as well as questions related to the ability of cytokines to prevent allograft (vs. autoimmune) rejection, both immunosuppressed and non-immunosuppressed arms have been added to these studies. This addition will provide information as to the importance and necessity of allowing for protective levels of transgene expression to avoid immune rejection within the early transplant period (i.e., 21 days). Alternatively, if short prolongation of islet grafts in diabetic NOD mice is observed in our experiments as previously reported by others (Markees et al., 1999), we will use an adoptive transfer system to overcome the delayed expression of cytokines by rAAV. To do this, transfected islets will be transplanted into chemically diabetic NOD-scid mice. Three to four weeks later, spleen cells will be adoptively transferred from diabetic NOD mice. Following baseline evaluation, serum samples are collected from animals (pooled when necessary) of these animals on a weekly basis and assayed serologically. Animals are monitored 3 times a week for hyperglycemia; with life-table analysis of the rate to recurrent Type I diabetes determined. At the onset of disease or at 120 days (in the case of non-diabetic animals), mice are sacrificed and examined. For studies in vitro (i.e., defined below), adenovirus co-infection may be used in order to amplify transgene expression.

5.3.8 Production of rAAV/Dose/Assessment of Transgene Production

An exemplary promoter used for constitutive expression of cytokines is the CMV immediate early (CMVp) promoter, the insulin promoter, or a CMV enhancer/β-actin promoter (CBAp); the latter showing recent evidence of markedly enhanced duration of transgene expression. Islet cells are transduced with multiplicities of infection (moi) ranging from 4×10⁵ to 4×10⁶ particles per cell. Secretion of the relevant cytokine into medium or serum is assessed by antigen-capture ELISA (Murphy et al., 1997).

### 5.3.9 Islet Transplantation

Islets from donor mice are transduced ex vivo with rAAV-IL-4 or rAAV-AAT and, 24 hr later, transplanted to animals. Monitoring of graft function and diabetes recurrence is obtained by measurement of blood glucose levels, with diabetes occurrence defined as at least two consecutive readings higher than 240 mg/dl.

### 5.3.10 Immunosuppression

An induction dose of 20 mg/kg (MR1) of anti-CD154 ab is administered intraperitoneally on days −1, 0 and 3, day 0 being the day of transplantation. An identical maintenance dose (20 mg/kg) is administered on day 7, and every week thereafter until day 21.

### 5.3.11 Analysis of Renal Subcapsular Grafts

Analysis of graft-bearing kidneys is performed by conventional histology and immunohistochemistry. H&E staining is used for routine morphological analysis. Staining with hormone-specific (insulin, glucagon, and somatostatin) and leukocyte lineage-specific antibodies (CD4, CD8, CD3, CD16, and Mac-3) defines the specificity and subset participation to rejection/survival of grafts.

### 5.3.12 In Vitro Analysis of Lymphocyte Proliferation and Cytokine Production

Splenic lymphocytes are obtained by animals at the time of sacrifice and utilized in standard mixed leukocyte reactions and mitogen stimulation assays.

### 5.3.13 Analysis of Intragraft and Serum Cytokine Expression

Graft bearing kidneys are harvested at the time of sacrifice of the animals, diabetes recurrence, or at 120 days. mRNA is extracted (avoiding parenchyma) and quantitative analysis of cytokine steady state levels performed utilizing our TaqMan (Perkin Elmer) system with primers for murine IL1, IL2, IL4, IFNγ, TNFα and IL10. Serum cytokine (IL-4) are also measured.

### 5.3.14 Analysis of Apoptosis

The occurrence of apoptosis in transplanted tissue is assessed by TUNEL assay for the detection of fragmented DNA. Double fluorescence analysis with hormone specific antibodies allows the definition of cell subsets undergoing apoptosis in the graft (i.e., β, α, δ).

### 5.3.15 In Vitro Assessment of Islet Resistance to T Cell Destruction

NOD islets are exposed in vitro to sort purified CD8+ T cells obtained from NOD.AI4αβ Tg) mice (Gallichan et al., 1998) to evaluate resistance to destruction in vitro. CTL activity (w/wo 100 ng/ml of IFN-γ) is assessed by chromium release, apoptosis of islet cells by FACS analysis of Annexing V binding, and inhibition of AI4 cell proliferation (H³-thymidine assays; IL-2 and IFN-γ production).

### 5.3.16 Total Ig Subclass, Isotype, and Insulin Autoantibody Levels

Total mouse Ig subclass and isotype are quantitated with kits purchased from The Binding Site (San Diego, Calif.). Autoantibodies to insulin, as well as isotype/subclass, are measured by micro RIA as previously described (Rendahl et al., 1998).

5.3.17 rAAV Transduction of Human and Mouse Islet Cells

Human and mouse islets were isolated using collagenase, purified by gradient selection (human) or centrifugation/hand picking (mouse), pretreated with Ad 5 (5 infectious units (IU) per cell; used to accelerate transgene expression in short term in vitro analyses), and transduced at multiplicities of infection (moi) of 0 to 10,000 IU/cell with rAAV-GFP constructs utilizing either the cytomegalovirus (CMV) promoter enhancer or the CMV enhancer-chicken beta actin (CB) hybrid promoter (CB). Transduction efficiencies ranged from 1% at an moi of 400 iu/cell to at least 35% at an moi of 4000 iu/cell, with similar efficiencies with either CMV or CB vectors.

5.3.18 Secretion of Cytokines by Human Islet Cells Following rAAV Transduction

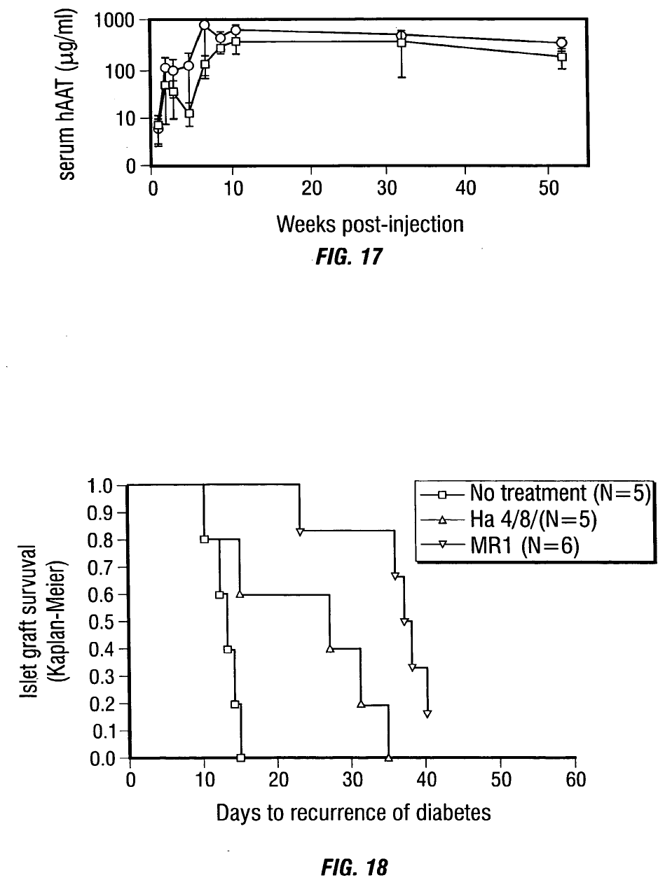

Studies were performed with rAAV-CMV-IL-4 and rAAV-CMV-IL-10 vectors under similar conditions (in triplicate). IL-4 was secreted from treated islets at a concentration of 2.23±0.74 ng/ml while IL-10 levels were 1.62±0.11 ng/ml. These data demonstrate efficient rAAV-mediated islet cell transduction using either of these cytokines (FIG. 17).

5.3.19 Transduction of Murine Islet Cells with Bicistronic AAV Vector Containing GFP and RFP Demonstrate Simultaneous Expression of Two Transgenes within β Cells

A bicistronic AAV vector that encompasses a viral IRES allowing for translation of two cDNAs (e.g., GFP and RFP) from a single mRNA transcript has been developed and tested for efficacy in the present system. Confocal microscopy utilizing separate excitation wavelengths for both proteins demonstrated co-expression and co-localization of both reporter proteins.

### 5.3.20 Natural History of Recurrent Autoimmunity and Type I Diabetes in Immunosuppressed and Non-Immunosuppressed Recipients

One aim of this study was to determine the effects of immunosuppression afforded by costimulatory blockade on the recurrence of autoimmunity. The model utilized was based on syngeneic islet transplantation in overtly diabetic female NOD mice. As shown in FIG. 18, a significant delay in autoimmunity recurrence was obtained in the treated group. However anti-CD154 treatment did not provide permanent protection from diabetes recurrence in this model system.

### 5.4 Example 4

### IM Injection of rAAV-Cytokine Constructs Completely Prevent Type I Diabetes in Mammals

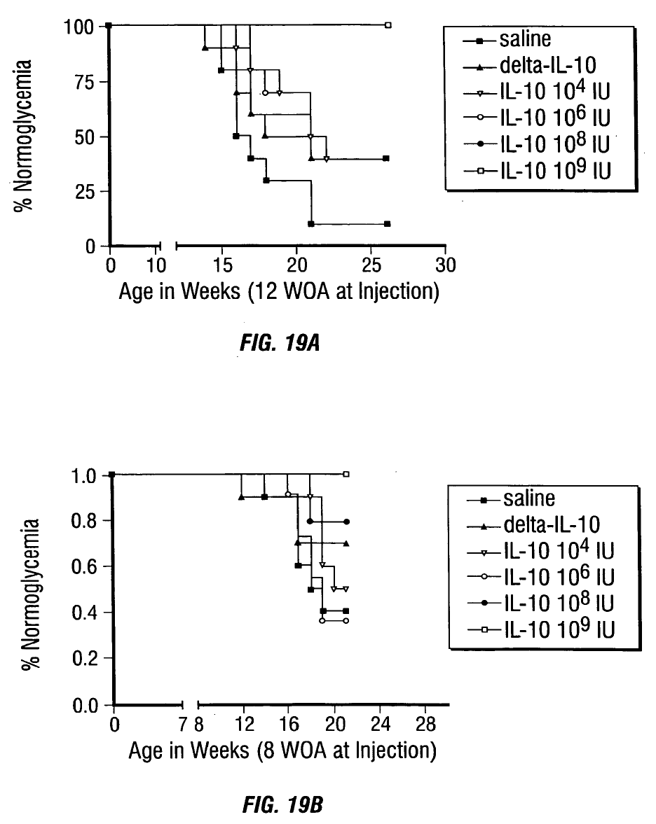

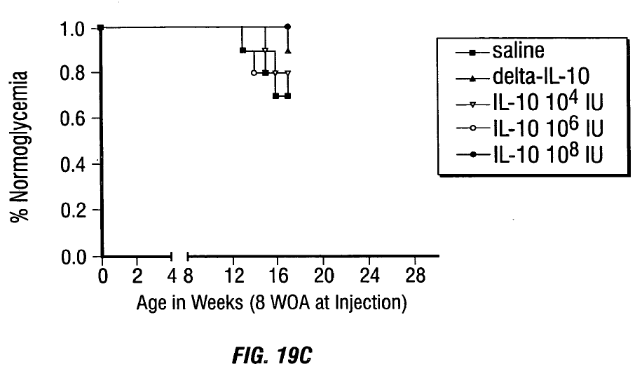

Female NOD mice were injected with CB-IL-10 (indicated doses) at 12 weeks of age (right before onset of diabetes) (FIG. 19A). In this study, rAAV-CMV-delta IL-10 (delta-IL-10), which mediates no IL-10 expression, served as control. Surprisingly, intramuscular injection of 10×9 i.u. of CB-IL-10 at 12 week of age completely prevents Type I diabetes. FIG. 19B and FIG. 19C show data from studies in which female NOD mice were injected the same vectors at 8 (FIG. 19B) or 4 (FIG. 19C) weeks of age.

### 5.5 Illustrative Therapeutic Polypeptide Sequences Useful in the Practice of the Present Invention

| Human IL-10 Protein (GenBank # A38580) | | |
|---|---|---|
| (SEQ ID NO:1) | | |
| MHSSALLCCLVLLTGVRASPGQGTQSENSCTHFPGNLPNMLRDLRDAFSRVKTFFQMKDQLD | | |
| NLLLKESLLEDFKGYLGCQALSEMIQFYLEEVMPQAENQDPDIKAHVNSLGENLKTLRLRLR | | |
| RCHRFLPCENKSKAVEQVKNAFNKLQEKGIYKAMSEFDIFINYIEAYMTMKIRN | | |
| Human IL-6 Protein (GenBank # IVHUB2) | | |
| (SEQ ID NO:2) | | |
| MNSFSTSAFGPVAFSLGLLLVLPAAFPAPVPPGEDSKDVAAPHRQPLTSSERIDKQIRYILD | | |
| GISALRKETCNKSNMCESSKEALAENNLNLPKMAEKDGCFQSGFNEETCLVKIITGLLEFEV | | |
| YLEYLQNRFESSEEQARAVQMSTKVLIQFLQKKAKNLDAITTPDPTTNASLLTKLQAQNQWL | | |
| QDMTTHLILRSFKEFLQSSLRALRQM | | |
| Human IL-4 Protein (GenBank # A25946) | | |
| (SEQ ID NO:3) | | |
| MGLTSQLLPPLFFLLACAGNFVHGHKCDITLQEIIKTLNSLTEQKTLCTELTVTDIFAASKN | | |
| TTEKETFCRAATVLRQFYSHHEKDTRCLGATAQQFHRHKQLIRFLKRLDRNLWGLAGLNSCP | | |
| VKEANQSTLENFLERLKTIMREKYSKCSS | | |
| Human Elafin Protein (GenBank # AAB26371) | | |
| (SEQ ID NO:4) | | |
| MRASSFLIVVVFLIAGTLVLEAAVTGVPVKGQDTVKGRVPFNGQDPVKGQVSVKGQDKVKAQ | | |
| EPVKGPVSTKPGSCPIILIRCAMLNPPNRCLKDTDCPGIKKCCEGSCGMACFVPQ | | |
| Human Elafin-like Protein | | |
| (SEQ ID NO:5) | | |
| MTQPGVLRSAAARKPGYCPEFDLDCPFTLLPMRWRDKSCRGSRSVATTTVGISVWSPGGLWI | | |
| EVRSYPLCKSFEERSYPFCESFKDQQTSEHPACREEPPSPGPPLC | | |
| Macaque sTrappin-2 (GenBank # CAA11183) | | |
| (SEQ ID NO:6) | | |
| VVVFLIAGMLVVEAAVTGVPVKGQDTVKGRVPFNGQDPVKGQVSVKGQDRVKGRGPVKGPVS | | |
| TKPGSCPNILIRCAMLNPPNRCLKDTD | | |
| Pig Elafin-like Protein (GenBank # BAA08857) | | |
| (SEQ ID NO:7) | | |
| MRSRSFLVLVVVFLICGTLVVQAAGRIRRPKGKGTKKTLALVKGQGPVRGKDQVKGQGPVKG | | |
| QDLGKSQDPVKAQLPDKGQDPVKAQPAIKRLILLTKPGSCPRILIRCLMVNPPNRCLSDAQC | | |
| PGVKKCCEGFCGKDCMDPK | | |
| Pig Elafin Precursor Protein (WAP-1) (GenBank #Q29125) | | |
| (SEQ ID NO:8) | | |
| MRSRSFLVLVVVFLICGTLVAQAAGRIRRPKGKGTKKILALVKGQGPVRGKDQVKGQGPVKG | | |
| QDLGKSQDPVKAQLPDKGQDLGKGEDSVKGQDPFKAQLPDKLQDPVKAQPAIKRLILLTKPG | | |
| SCPRILIRCLMVNPPNRCLSDAQCPGLKKCCEGFCGKACMDPK | | |
| Bovine Trappin-6 Protein (GenBank # JE0252) | | |
| (SEQ ID NO:9) | | |
| SPKGQGNVVFNGKGPVNGQSPDKGQDPVKGQDPVKGQDVVVAQDRAGLPFKRGLCPRVRIHC | | |
| NLWNPPNQCWRDAHCPGAKKCCEGFCGKTCMNPR | | |
| Rat SLPI Protein (GenBank # AAD51758) | | |
| (SEQ ID NO:10) | | |
| MKSCGLFPLMVLLALGVLAPWSVEGGKNDAIKIGACPARKPAQCLKLEKPECGTDWECPGKQ | | |
| RCCQDTCGFKCLNPVPIRGPVKKKPGRCVKFQGKCLMLNPPNKCQNDGQCDGKYKCCEGMCG | | |
| KVCLPPV | | |
| Mouse SLPI Protein (GenBank #AAC53047) | | |
| (SEQ ID NO:11) | | |
| MKSCGLLPFTVLLALGILAPWTVEGGKNDAIKIGACPAKKPAQCLKLEKPQCRTDWECPGKQ | | |
| RCCQDACGSKCVNPVPIRKPVWRKPGRCVKTQARCMMLNPPNVCQRDGQCDGKYKCCEGICG | | |
| KVCLPPM | | |
| Protein | Rat glia-derived nexin I alpha Precursor | |
| PIR | Name: A27496 | |
| NCBI | seq ID: 87514 | |
| Citation | J. Sommer, S. M. Gloor, G. F. Rovelli, J. Hofsteenge, H. | |
| | Nick, R. Meier & D. Monard (1987). cDNA sequence coding | |
| | for a rat glia-derived nexin and its homology to members | |
| | of the serpin superfamily. Biochemistry 26, 6407-6410. | |
| | MEDLINE identifier: 88107544 | |
| domain | signal sequence | 87514: 1 . . . 19 |
| product | glia-derived nexin I alpha | 87514: 20 . . . 397 |
| Sequence | 397 aa | |
| (SEQ ID NO:12) | | |
| 1 | MNWHLPLFLLASVTLPSICSHFNPLSLEELGSNTGIQVFNQIVKSRPHDN | |
| 51 | IVISPHGIASVLGMLQLGADGRTKKQLAMVMRYGVNGVGKILKKINKAIV | |
| 101 | SKKNKDIVTVANAVFVKNASEIEVPFVTRNKDVFQCEVRNVNFEDPASAC | |
| 151 | DSINAWVKNETRDMIDNLLSPDLIDGVLTRLVLVNAVYFKGLWKSRFQPE | |
| 201 | NTKKRTFVAADGKSYQVPMLAQLSVFRCGSTSAPNDLWYNFIELPYHGES | |
| 251 | ISMLIALPTESSTPLSAIIPHISTKTIDSWMSIMVPKRVQVILPKFTAVA | |
| 301 | QTDLKEPLKVLGITDMFDSSKANFAKITRSENLHVSHILQKAKIEVSEDG | |
| 351 | TKASAATTAILIARSSPPWFIVDRPFLFFIRHNPTGAVLFMGQINKP | |
| Protein | serine proteinase inhibitor | |
| NCBI | Seq ID: 2104735 | |
| Citation | J Sun, L Ooms, C Bird, V Sutton, J Trapani & P Bird | |
| | (1997). A new family of ten murine ovalbumin serpins | |
| | includes Two homologs of proteinase inhibitor 8 and two | |
| | homologs of the granzyme B inhibitor (proteinase | |
| | inhibitor 9). J. Biol. Chem. | |
| Sequence | 374 aa | |
| (SEQ ID NO:13) | | |
| 1 | MNTLSEGNGTFAIHLLKMLCQSNPSKNVCYSPASISSALAMVLLGAKGQT | |
| 51 | AVQISQALGLNKEEGIHQGFQLLLRKLNKPDRKYSLRVANRLFADKTCEV | |
| 101 | LQTFKESSLHFYDSEMEQLSFAEEAEVSRQHINTWVSKQTEGKIPELLSG | |
| 151 | GSVDSETRLVLINALYFKGKWHQPFNKEYTMDMPFKINKDEKRPVQMMCR | |
| 201 | EDTYNLAYVKEVQAQVLVMPYEGMELSLVVLLPDEGVDLSKVENNLTFEK | |
| 251 | LTAWMEADFMKSTDVEVFLPKFKLQEDYDMESLFQRLGVVDVFQEDKADL | |
| 301 | SGMSPERNLCVSKFVHQSVVEINEEGTEAAAASAIIEFCCASSVPTFCAD | |
| 351 | HPFLFFIRHNKANSILFCGRFSSP | |
| Protein | serine proteinase inhibitor Common Carp Serpin | |
| PIR | Name: I50494 | |
| NCBI | Seq ID: 2133935 | |
| Citation | C. J. Huang, M. S. Lee, F. L. Huang & G. D. Chang (1995). | |
| | A protease inhibitor of the serpin family is a major | |
| | protein in carp perimeningeal fluid: II. cDNA cloning, | |
| | sequence analysis, and Escherichia coli expression. J. | |
| | Neurochem. 64, 1721-1727. MEDLINE identifier: 95198028 | |
| Sequence | 410 aa | |
| (SEQ ID NO:14) | | |
| 1 | MAWAAPHEGHDHDGHPADHYHHLHHGKDEAHPSHSGEDACHLLSPHNADF | |
| 51 | AFSLYKKLALHPDAQGKNIFFSPVGISMALSMLAVGAKGSTLSQIYSSLG | |
| 101 | YSGLKAQQVNEGYEHLIHMLGHSQDTMQLEAGAGVAIREGFKVVDQFLKD | |
| 151 | VQHYYNSEAFSVDFSKPEIAAEEINQFIAKKTNDKITDMVKDLDSDMVMM | |
| 201 | LINYMYFRGKWDKPFEAQLTHKAEFKVDKDTTVQVDMMKRTGRYDIYQDP | |
| 251 | VNQTTVMMVPYKGNTSMMIVLPDEGKMKDVEESICRRHLKNWHDKLFRSS | |
| 301 | VDLFMPKFSISATSKLNDILTEMGVTDAFSDTADFSGMTEELKVKVSQVV | |
| 351 | HKAVLSVDEKGTEAAAATTIEIMPMSLPGTVMLNRPFLVLIVEDTTKSIL | |
| 401 | FMGKITNPTV | |
| Protein | Pig serpin | |
| PIR | Name: S38962 | |
| NCBI | Seq ID: 481621 | |
| Citation | W. F. Teschauer, R. Mentele & C. P. Sommerhoff (1993). | |
| | Primary structure of a porcine leukocyte serpin. Eur. J. | |
| | Biochem. 217, 519-526. MEDLINE identifier: 94039085 | |
| Sequence | 378 aa | |
| (SEQ ID NO:15) | | |
| 1 | MEQLSAANTRFALDLFRALNESNPAGNIFISPFSISSALAMILLGTRGNT | |
| 51 | EAQMSKALHFDTVKDIHSRFQSLNADINKCGASYILKLANRLFGEKTYHF | |
| 101 | LPEFLASTQKTYGAELASVDFLRASEEARKAINEWVKEQTEGKIPELLAS | |
| 151 | GVVDSATKLVLVNAIYFKGSWQEKFMTEATKDAPFRLNKKDSKTVKMMYQ | |
| 201 | KKKFPFGYIKELKCRVLELPYQGKDLSMVILLPDSIEDESTGLRKIEQHL | |
| 251 | TLEKLREWTKPDNLELLEVNVHLPRFRLEESYDLNAPLARLGVQDLFGSR | |
| 301 | ADLTGMSEARDLFISKVVHKSFVEVNEEGTEAAAATXGIAVFAMLMPEED | |
| 351 | FIADHPFIFFIRHNPSSNILFLGRLSSP | |
| Protein | Horse serpin | |
| PIR | Name: S25828 | |
| NCBI | Seq ID: 108207 | |
| Citation | J. Potempa, J. K. Wunderlich & J. Travis (1991). Compara- | |
| | tive properties of three functionally different but | |
| | structurally related serpin variants from horse plasma. | |
| | Biochem. J. 274, 465-471. MEDLINE identifier: 91174757 | |
| Sequence | 54 aa | |
| (SEQ ID NO:16) | | |
| 1 | EDLQGDAVPERHATKDDNEHPQEPAEHKKAPNEAIRTLLHTNVEFNRPFV | |
| 51 | LIIY | |
| Protein | Horse serpin | |
| PIR | Name: S25829 | |
| NCBI | Seq ID: 108206 | |
| Citation | J. Potempa, J. K. Wunderlich & J. Travis (1991). Compara- | |
| | tive properties of three functionally different but | |
| | structurally related serpin variants from horse plasma. | |
| | Biochem. J. 274, 465-471. MEDLINE identifier: 91174757 | |
| Sequence | 49 aa | |
| (SEQ ID NO:17) | | |
| 1 | EDLQGDAVPERHATKDDNEHPQEPAEHKKAPNERPATLLLDNVEFNRPF | |
| Protein | Horse serpin | |
| PIR | Name: S14338 | |
| NCBI | Seq ID: 108205 | |
| Citation | J. Potempa, J. K. Wunderlich & J. Travis (1991). Compara- | |
| | tive properties of three functionally different but | |
| | structurally related serpin variants from horse plasma. | |
| | Biochem. J. 274, 465-471. MEDLINE identifier: 91174757 | |
| Sequence | 54 aa | |
| (SEQ ID NO:18) | | |
| 1 | EDLQGDAVPERHATKDDNEHPQEPAEHKKAPNEMIPMSLPPELEFNRPFI | |
| 51 | LIIY | |
| Protein | human leupin fragment | |
| PIR | Name: S57522 | |
| NCBI | Seq ID: 1362853 | |
| Citation | R. C. Barnes & D. M. Worrall (1995). Identification of a | |
| | novel human serpin gene; cloning sequencing and ex- | |
| | pression of leupin. FEBS Lett. 373, 61-65. MEDLINE | |
| | identifier: 96013887 | |
| Sequence | 390 aa | |
| (SEQ ID NO:19) | | |
| 1 | MNSLSEANTKFMFDLFQQFRKSKENNIFYSPISITSALGMVLLGAKDNTA | |
| 51 | QQISKVLHFDQVTENTTEKAATYHVDRSGNVHHQFQKLLTEFNKSTDAYE | |
| 101 | LKIANKLFGEKTYQFLQEYLDAIKKFYQTSVESTDFANAPEESRKKINSW | |
| 151 | VESQTNEKIKNLFPDGTIGNDTTLVLVNAIYFKGQWENKFKKENTKEEKF | |
| 201 | WPNKNTYKSVQMMRQYNSFNFALLEDVQAKVLEIPYKGKDLSMIVLLPNE | |
| 251 | IDGLQKLEEKLTAEKLMEWTSLQNMRETCVDLHLPRFKMEESYDLKDTLR | |
| 301 | TMGMVNIFNGDADLSGMTWSHGLSVSKVLHKAFVEVTEEGVEAAAATAVV | |
| 351 | VVELSSPSTNEEFCCNHPFLFFIRQNKTNSILFYGRFSSP | |
| Protein | human alpha-1-antitrypsin precursor; α-1-AT; α-1-pro- | |
| | teinase inhibitor | |
| PIR | Name: ITHU | |
| NCBI | Seq ID: 68741 | |
| Citation | G. L. Long, T. Chandra, S. L. Woo, E. W. Davie & K. | |
| | Kurachi (1984). Complete sequence of the cDNA for human | |
| | α1-antitrypsin and the gene for the S variant. Bio- | |
| | chemistry 23, 4828-4837. MEDLINE identifier: 85047190 | |
| domain | signal sequence | 68741: 1 . . . 24 |
| product | alpha-1-antitrypsin | 68741: 25 . . . 418 |
| Sequence | 418 aa | |
| (SEQ ID NO:20) | | |
| 1 | MPSSVSWGILLLAGLCCLVPVSLAEDPQGDAAQKTDTSHHDQDHPTFNKI | |
| 51 | TPNLAEFAFSLYRQLAHQSNSTNIFFSPVSIATAFAMLSLGTKADTHDEI | |
| 101 | LEGLNFNLTEIPEAQIHEGFQELLRTLNQPDSQLQLTTGNGLFLSEGLKL | |
| 151 | VDKFLEDVKKLYHSEAFTVNFGDTEEAKKQINDYVEKGTQGKIVDLVKEL | |
| 201 | DRDTVFALVNYIFFKGKWERPFEVKDTEEEDFHVDQVTTVKVPMMKRLGM | |
| 251 | FNIQHCKKLSSWVLLMKYLGNATAIFFLPDEGKLQHLENELTHDIITKFL | |
| 301 | ENEDRRSASLHLPKLSITGTYDLKSVLGQLGITKVFSNGADLSGVTEEAP | |
| 351 | LKLSKAVHKAVLTIDEKGTEAAGAMFLEAIPMSIPPEVKFNKPFVFLMIE | |
| 401 | QNTKSPLFMGKVVNPTQK | |
| Protein | human antithrombin III precursor | |
| PIR | Name: XHHU3 | |
| NCBI | Seq ID: 68734 | |
| Citation | R. J. Olds, D. A. Lane, V. Chowdhury, V. De Stefano, G. | |
| | Leone & S. L. Thein (1993). Complete nucleotide sequence | |
| | of the antithrombin gene: evidence for homologous | |
| | recombination causing thrombophilia. Biochemistry 32, | |
| | 4216-4224. MEDLINE identifier: 93237227 | |
| domain | signal sequence | 68734: 1 . . . 32 |
| product | antithrombin III | 68734: 33 . . . 464 |
| Sequence | 464 aa | |
| (SEQ ID NO:21) | | |
| 1 | MYSNVIGTVTSGKRKVYLLSLLLIGFWDCVTCHGSPVDICTAKPRDIPMN | |
| 51 | PMCIYRSPEKKATEDEGSEQKIPEATNRRVWELSKANSRFATTFYQHLAD | |
| 101 | SKNDNDNIFLSPLSISTAFAMTKLGACNDTLQQLMEVFKFDTISEKTSDQ | |
| 151 | IHFFFAKLNCRLYRKANKSSKLVSANRLFGDKSLTFNETYQDISELVYGA | |
| 201 | KLQPLDFKENAEQSRAAINKWVSNKTEGRITDVIPSEAINELTVLVLVNT | |
| 251 | IYFKGLWKSKFSPENTRKELFYKADGESCSASMMYQEGKPRYRRVAEGTQ | |
| 301 | VLELPFKGDDITMVLILPKPEKSLAKVEKELTPEVLQEWLDELEEMMLVV | |
| 351 | HMPRFRIEDGFSLKEQLQDMGLVDLFSPEKSKLPGIVAEGRDDLYVSDAF | |
| 401 | HKAFLEVNEEGSEAAASTAVVIAGRSLNPNRVTFKANRPFLVFIREVPLN | |
| 451 | TIIFMGRVANPCVK | |
| Protein | chicken serpin precursor | |
| PIR | Name: S70647 | |
| NCBI | Seq ID: 2134403 | |
| Citation | T. Osterwalder, J. Contartese, E. T. Stoeckli, T. B. | |
| | Kuhn & P. Sonderegger (1996). Neuroserpin, an axonally | |
| | secreted serine protease inhibitor. EMBO J. 15, 2944- | |
| | 2953. MEDLINE identifier: 96272154 | |
| Sequence | 410 aa | |
| (SEQ ID NO:22) | | |
| 1 | MYFLGLLSLLVLPSKAFKTNFPDETIAELSVNVYNQLRAAREDENILFCP | |
| 51 | LSIAIAMGMIELGAHGTTLKEIRHSLGFDSLKNGEEFTFLKDLSDMATTE | |
| 101 | ESHYVLNMANSLYVQNGFHVSEKFLQLVKKYFKAEVENIDFSQSAAVATH | |
| 151 | INKWVENHTNNMIKDFVSSRDFSALTHLVLINAIYFKGNWKSQFRPENTR | |
| 201 | TFSFTKDDETEVQIPMMYQQGEFYYGEFSDGSNEAGGIYQVLEIPYEGDE | |
| 251 | ISMMIVLSRQEVPLVTLEPLVKASLINEWANSVKKQKVEVYLPRFTVEQE | |
| 301 | IDLKDVLKGLGITEVFSRSADLTAMSDNKELYLAKAFHKAFLEVNEEGSE | |
| 351 | AAAASGMIAISRMAVLYPQVIVDHPFFFLVRNRRTGTVLFMGRVMHPEAM | |
| 401 | NTSGHDFEEL | |
| Protein | murine beta-glucuronidase precursor; beta-D-glucuronoside | |
| | glucuronosohydrolase | |
| PIR | Name: A29977 | |
| NCBI | Seq ID: 90328 | |
| Citation | M. A. D'Amore, P. M. Gallagher, T. R. Korfhagen & R. E. | |
| | Ganschow (1988). Complete sequence and organization of | |
| | the murine beta-glucuronidase gene. Biochemistry 27, | |
| | 7131-7140. MEDLINE identifier: 89062453 | |
| domain | signal sequence | 90328: 1 . . . 22 |
| product | beta-glucuronidase | 90328: 23 . . . 648 |
| Sequence | 648 aa | |
| (SEQ ID NO:23) | | |
| 1 | MSLKWSACWVALGQLLCSCALALKGGMLFPKESPSRELKALDGLWHFRAD | |
| 51 | LSNNRLQGFEQQWYRQPLRESGPVLDMPVPSSFNDITQEAALRDFIGWVW | |
| 101 | YEREAILPRRWTQDTDMRVVLRINSAHYYAVVWVNGIHVVEHEGGHLPFE | |
| 151 | ADISKLVQSGPLTTCRITIAINNTLTPHTLPPGTIVYKTDTSMYPKGYFV | |
| 201 | QDTSFDFFNYAGLHRSVVLYTTPTTYIDDITVITNVEQDIGLVTYWISVQ | |
| 251 | GSEHFQLEVQLLDEDGKVVAHGTGNQGQLQVPSANLWWPYLMHEHPAYMY | |
| 301 | SLEVKVTTTESVTDYYTLPVGIRTVAVTKSKFLINGKPFYFQGVNKHEDS | |
| 351 | DIRGKGFDWPLLVKDFNLLRWLGANSFRTSHYPYSEEVLQLCDRYGIVVI | |
| 401 | DECPGVGIVLPQSFGNESLRHHLEVMEELVRRDKNHPAVVMWSVANEPSS | |
| 451 | ALKPAAYYFKTLITHTKALDLTRPVTFVSNAKYDADLGAPYVDVICVNSY | |
| 501 | FSWYHDYGHLEVIQPQLNSQFENWYKTHQKPIIQSEYGADAIPGIHEDPP | |
| 551 | RMFSEEYQKAVLENYHSVLDQKRKEYVVGELIWNFADFMTNQSPLRVIGN | |
| 601 | KKGIFTRQRQPKTSAFILRERYWRIANETGGHGSGPRTQCFGSRPFTF | |
| Protein | plasminogen activator inhibitor 2 precursor; urokinase | |
| | inhibitor; human | |
| PIR | Name: A32853 | |
| NCBI | Seq ID: 107324 | |
| Citation | R. D. Ye, S. M. Ahern, M. M. Le Beau, R. V. Lebo & J. E. | |
| | Sadler (1989). Structure of the gene for human plasmino- | |
| | gen activator inhibitor-2. The nearest mammalian homo- | |
| | logue of chicken ovalbumin. J. Biol. Chem. 264, 5495- | |
| | 5502. MEDLINE identifier: 89174589 | |
| domain | signal sequence | 107324: 1 . . . 22 |
| product | plasminogen activator inhibitor 2 | 107324: 23 . . . 415 |
| Sequence | 415 aa | |
| (SEQ ID NO:24) | | |
| 1 | MEDLCVANTLFALNLFKHLAKASPTQNLFLSPWSISSTMAMVYMGSRGST | |
| 51 | EDQMAKVLQFNEVGANAVTPMTPENFTSCGFMQQIQKGSYPDAILQAQAA | |
| 101 | DKIHSSFRSLSSAINASTGNYLLESVNKLFGEKSASFREEYIRLCQKYYS | |
| 151 | SEPQAVDFLECAEEARKKINSWVKTQTKGKIPNLLPEGSVDGDTRMVLVN | |
| 201 | AVYFKGKWKTPFEKKLNGLYPFRVNSAQRTPVQMMYLREKLNIGYIEDLK | |
| 251 | AQILELPYAGDVSMFLLLPDEIADVSTGLELLESEITYDKLNKWTSKDKM | |
| 301 | AEDEVEVYIPQFKLEEHYELRSILRSMGMEDAFNKGRANFSGMSERNDLF | |
| 351 | LSEVFHQAMVDVNEEGTEAAAGTGGVMTGRTGHGGPQFVADHPFLFLIMH | |
| 401 | KITNCILFFGRFSSP | |
| Protein | alpha-1-antiproteinase isoform E precursor; rabbit | |
| PIR | Name: S54981 | |
| NCBI | Seq ID: 2118396 | |
| Citation | A. Saito & H. Sinohara (1995). Rabbit alpha-1-anti- | |
| | proteinase E: a novel recombinant serpin which does not | |
| | inhibit proteinases. Biochem. J. 307, 369-375. MEDLINE | |
| | identifier: 95251597 | |
| domain | signal sequence | 2118396: 1 . . . 24 |
| product | α-1-antiproteinase E | 2118396: 25 . . . 413 |
| Sequence | 413 aa | |
| (SEQ ID NO:25) | | |
| 1 | MPPSVSRALLLLAGLGCLLPGFLADEAQETAVSSHEQDHPACHRIAPSLA | |
| 51 | EFALSLYREVAHESNTTNIFFSPVSIALAFAMLSLGAKGDTHTQVLEGLK | |
| 101 | FNLTETAEAQIHDGFRHLLHTVNRPDSELQLAAGNALVVHENLKLQHKFL | |
| 151 | EDAKNLYQSEAFLVDFRDPEQAKTKINSHVEKGTRGKIVDLVQELDARTL | |
| 201 | LALVNYVFFKGKWEKPFEPENTKEEDFHVDATTTVRVPMMSRLGMYVMFH | |
| 251 | CSTLASTVLRMDYKGNATALFLLPDEGKLQHLEDTLTTELIAKFLAKSSL | |
| 301 | RSVTVRFPKLSISGTYDLKPLLGKLGITQVFSNNADLSGITEQEPLKVSQ | |
| 351 | ALHKAVLTIDERGTEAAGASFVELIPESVPDSITLDRPFLFVIYSHEIKS | |
| 401 | PLFVGKVVDPTQH | |
| Protein | Rat glia-derived nexin precursor92273: [Whole] | |
| PIR | Name: B27496 | |
| NCBI | Seq ID: 92273 | |
| Citation | J. Sommer, S. M. Gloor, G. F. Rovelli, J. Hofsteenge, H. | |
| | Nick, R. Meier & D. Monard (1987). cDNA sequence coding | |
| | for a rat glia-derived nexin and its homology to members | |
| | of the serpin superfamily. Biochemistry 26, 6407-6410. | |
| | MEDLINE identifier: 88107544 | |
| domain | signal sequence | 92273: 1 . . . 19 |
| product | glia-derived nexin | 92273: 20 . . . 397 |
| Sequence | 397 aa | |
| (SEQ ID NO:26) | | |
| 1 | MNWHFPFFILTTVTLSSVYSQLNSLSLEELGSDTGIQVFNQIIKSQPHEN | |
| 51 | VVISPHGIASILGMLQLGADGRTKKQLSTVMRYNVNGVGKVLKKINKAIV | |
| 101 | SKKNKDIVTVANAVFVRNGFKVEVPFAARNKEVFQCEVQSVNFQDPASAC | |
| 151 | DAINFWVKNETRGMIDNLLSPNLIDSALTKLVLVNAVYFKGLWKSRFQPE | |
| 201 | NTKKRTFVAGDGKSYQVPMLAQLSVFRSGSTKTPNGLWYNFIELPYHGES | |
| 251 | ISMLIALPTESSTPLSAIIPHISTKTINSWMNTMVPKRMQLVLPKFTALA | |
| 301 | QTDLKEPLKALGITEMFEPSKANFAKITRSESLHVSHILQKAKIEVSEDG | |
| 351 | TKAAVVTTAILIARSSPPWFIVDRPFLFCIRHNPTGAILFLGQVNKP | |
| Protein | Pig uteroferrin-associated protein precursor | |
| PIR | Name: A34722 | |
| NCBI | Seq ID: 89313 | |
| Citation | P. V. Malathy, K. Imakawa, R. C. Simmen & R. M. Roberts | |
| | (1990). Molecular cloning of the uteroferrin-associated | |
| | protein, a major progesterone-induced serpin secreted by | |
| | the porcine uterus, and the expression of its mRNA during | |
| | pregnancy. Mol. Endocrinol. 4, 428-440. MEDLINE identi- | |
| | fier: 90258936 | |
| domain | signal sequence | 89313: 1 . . . 25 |
| product | uteroferrin-associated protein | 89313: 26 . . . 417 |
| Sequence | 417 aa | |
| (SEQ ID NO:27) | | |
| 1 | MSHGKMPLVLSLVLILCGLFNSISCEKQQTSPKTITPVSFKRIAALSQKM | |
| 51 | EANYKAFAQELFKTLLIEDPRKNMIFSPVSISISLATLSLGLRSATRTNA | |
| 101 | IDVLDVALKNLAVMLMAQAPTALLEIVHELVNRTAKHQDILIDRTEMNQM | |
| 151 | FLKEIDRYIKMDIQMIDFKDKEKTKKAINQFVADKIDKKAKNLITHLDPQ | |
| 201 | TLLCLVNYIFFKGILERAFQTNLTKKEDFFVNEKTIVQVDMMRKTERMIY | |
| 251 | SRSEELLATMVKIPCKENASIILVLPDTGKFNFALKEMAAKRARLQKTND | |
| 301 | FRLVRLVVPKIKDNLQDRFKHLLPKIGINDIFTTKAVTWNTTGTSTILEA | |
| 351 | VHHAVIEVKEDGLTKNAAKDKDFWKVPVDKKEVPVVVKFDRPFFLFVEDE | |
| 401 | ITRRDLFVAKVFNPKTE | |
| Protein | plasminogen activator inhibitor-1 precursor; PAI-1; | |
| | plasminogen activator inhibitor, endothelial-cell type; | |
| | human | |
| PIR | Name: ITHUP1 | |
| NCBI | Seq ID: 68735 | |
| domain | signal sequence | 68735: 1 . . . 23 |
| product | plasminogen activator inhibitor-1 | 68735: 24 . . . 402 |
| Sequence | 402 aa | |
| (SEQ ID NO:28) | | |
| 1 | MQMSPALTCLVLGLALVFGEGSAVHHPPSYVAHLASDFGVRVFQQVAQAS | |
| 51 | KDRNVVFSPYGVASVLAMLQLTTGGETQQQIQAAMGFKIDDKGMAPALRH | |
| 101 | LYKELMGPWNKDEISTTDAIFVQRDLKLVQGFMPHFFRLFRSTVKQVDFS | |
| 151 | EVERARFIINDWVKTHTKGMISNLLGKGAVDQLTRLVLVNALYFNGQWKT | |
| 201 | PFPDSSTHRRLFHKSDGSTVSVPMMAQTNKFNYTEFTTPDGHYYDILELP | |
| 251 | YHGDTLSMFIAAPYEKEVPLSALTNILSAQLISHWKGNMTRLPRLLVLPK | |
| 301 | FSLETEVDLRKPLENLGMTDMFRQFQADFTSLSDQEPLHVAQALQKVKIE | |
| 351 | VNESGTVASSSTAVIVSARMAPEEIIMDRPFLFVVRHNPTGTVLFMGQVM | |
| 401 | EP | |
| Protein | Horse leukocyte elastase inhibitor; plasminogen activator | |
| | inhibitor-2 homolog | |
| PIR | Name: A42421 | |
| NCBI | Seq ID: 284550 | |
| Citation | A. Dubin, J. Travis, J. J. Enghild & J. Potempa (1992). | |
| | Equine leukocyte elastase inhibitor. Primary structure | |
| | and identification as a thymosin-binding protein. J. | |
| | Biol. Chem. 267, 6576-6583. MEDLINE identifier: 92202200 | |
| Sequence | 379 aa | |
| (SEQ ID NO:29) | | |
| 1 | MEQLSTANTHFAVDLFRALNESDPTGNIFISPLSISSALAMIFLGTRGNT | |
| 51 | AAQVSKALYFDTVEDIHSRFQSLNADINKPGAPYILKLANRLYGEKTYNF | |
| 101 | LADFLASTQKMYGAELASVDFQQAPEDARKEINEWVKGQTEGKIPELLVK | |
| 151 | GMVDNMTKLVLVNAIYFKGNWQEKFMKEATRDAPFRLNKKDTKTVKMMYQ | |
| 201 | KKKFPYNYIEDLKCRVLELPYQGKELSMIILLPDDIEDESTGLEKIEKQL | |
| 251 | TLEKLREWTKPENLYLAEVNVHLPRFKLEESYDLTSHLARLGVQDLFNRG | |
| 301 | KADLSGMSGARDLFVSKIIHKSFVDLNEEGTEAAAATAGTIMLAMLMPEE | |
| 351 | NFNADHPFIFFIRHNPSANILFLGRFSSP | |
| Protein | heat shock protein precursor; chaperonin; collagen- | |
| | binding protein; colligin; human | |
| PIR | Name: S20608 | |
| NCBI | Seq ID: 105724 | |
| Citation | E. P. Clarke & B. D. Sanwal (1992). Cloning of a human | |
| | collagen-binding protein, and its homology with rat gp46, | |
| | chick hsp47 and mouse J6 proteins. Biochim. Biophys. Acta | |
| | 1129, 246-248. MEDLINE identifier: 92110393 | |
| domain | signal sequence | 105724: 1 . . . 17 |
| product | heat shock protein Hsp47 | 105724: 18 . . . 417 |
| Sequence | 417 aa | |
| (SEQ ID NO:30) | | |
| 1 | MRSLLLGTLCLLAVALAAEVKKPVEAAAPGTAEKLSSKATTLAEPSTGLA | |
| 51 | FSLYQAMAKDQAVENILVSPVVVASSLGLVSLGGKATTASQAKAVLSAEQ | |
| 101 | LRDEEVHAGLGELLRSLSNSTARNVTWKLGSRLYGPSSVSFADDFVRSSK | |
| 151 | QHYNCEHSKINFPDKRSALQSINEWAAQTTDGKLPEVTKDVERTDGALLV | |
| 201 | NAMFFKPHWDEKFHHKMVDNRGFMVTRSYTVGVTMMHRTGLYNYYDDEKE | |
| 251 | KLQLVEMPLAHKLSSLIILMPHHVEPLERLEKLLTKEQLKIWMGKMQKKA | |
| 301 | VAISLPKGVVEVTHDLQKHLAGLGLTEAIDKNKADLSRMSGKKDLYLASV | |
| 351 | FHATAFELDTDGNPFDQDIYGREELRSPKLFYADHPFIFLVRDTQSGSLL | |
| 401 | FIGRLVRLKGDKMRDEL | |
| Protein | COLLAGEN-BINDING PROTEIN 2 PRECURSOR; Human | |
| SWISS-PROT | Name: CBP2_HUMAN, Accession: P50454 | |
| NCBI | Seq ID: 1705664 | |
| Cross-ref | GenBank Accession: S79209 | |
| Citation | S. Ikegawa, K. Sudo, K. Okui & Y. Nakamura (1995). Isola- | |
| | tion, characterization and chromosomal assignment of | |
| | human colligin-2 gene (CBP2). Cytogenet. Cell Genet. 71, | |
| | 182-186. MEDLINE identifier: 95385381 | |
| Signal | | 1705664: 1 . . . 18 |
| Mature | COLLAGEN-BINDING PROTEIN 2. | 1705664: 19 . . . 418 |
| chain | | |
| Sequence | 418 aa | |
| (SEQ ID NO:31) | | |
| 1 | MRSLLLLSAFCLLEAALAAEVKKPAAAAAPGTAEKLSPKAATLAERSAGL | |
| 51 | AFSLYQAMAKDQAVENILVSPVVVASSLGLVSLGGKATTASQAKAVLSAE | |
| 101 | QLRDEEVHAGLGELLRSLSNSTARNVTWKLGSRLYGPSSVSFADDFVRSS | |
| 151 | KQHYNCEHSKINFRDKRRPLQSINEWAAQTTDGKLPEVTKDVERTDGALL | |
| 201 | VNAMFFKPHWDEKFHHKMVDNRGFMVTRSYTVGVMMMHRTGLYNYYDDEK | |
| 251 | EKLQIVEMPLAHKLSSLIILMPHHVEPLERLEKLLTKEQLKIWMGKMQKK | |
| 301 | AVAISLPKGVVEVTHDLQKHLAGLGLTEAIDKNKADLSRMSGKKDLYLAS | |
| 351 | VFHATAFELDTDGNPFDQDIYGREELRSPKLFYADHPFIFLVRDTQSGSL | |
| 401 | LFIGRLVRPKGDKMRDEL | |
| Protein | SHEEP ANGIOTENSINOGEN PRECURSOR 1703309: 1 . . . 476 | |
| SWISS-PROT | Name: ANGT_SHEEP, Accession: P20757 | |
| NCBI | Seq ID: 1703309 | |
| Citation | M. Nagase, F. Suzuki, A. Fukamizu, N. Takeda, K. | |
| | Takeuchi, K. Murakami & Y. Nakamura (1994). Sequencing | |
| | and expression of sheep angiotensinogen cDNA. Biosci. | |
| | Biotechnol. Biochem. 58, 1884-1885. MEDLINE identifier: | |
| | 95072318 | |
| Signal | (experimentally determined) | 1703309: 1 . . . 24 |
| Mature | ANGIOTENSINOGEN. | 1703309: 25 . . . 476 |
| chain | | |
| Processed | ANGIOTENSIN I. | 1703309: 25 . . . 34 |
| Processed | ANGIOTENSIN II. | 1703309: 25 . . . 32 |
| Sequence | 476 aa | |
| (SEQ ID NO:32) | | |
| 1 | MAPAGLSLGATILCLLAWAGLAAGDRVYIHPFHLLVHSKSNCDQLEKPSV | |
| 51 | ETPADPTLTPVPIQTKSSPVDEEALWEQLVRATEKLEAEDRLRASEVGLL | |
| 101 | LNFMGFHVYKTLSETWSVASGLVFSPVALFSTLTSFYTGALDPTASRLQA | |
| 151 | FLGVPGEGQGCTSRLDGRKVLSSLQTIQGLLVAPGGASSQARLLLSTVVG | |
| 201 | LFTAPGLHLKQPFVQGLSSFAPITLPRSLDLSTDPNLAAEKINRFMHSAT | |
| 251 | GWNMGRPLAAASPDSTLLFNAYVHFQGKMKGFSLLPGLTEFWVDNTTSVP | |
| 301 | VPMLSGSGTFHYWSDNQNHLSMTRVPLSANGYLLLIQPHHTLDLRKVEAL | |
| 351 | IFQHNFLTRMKNLSPRAIHLTVPQLTLKASYDLQDLLAQAKLPTLLGAEA | |
| 401 | NLGKISDANLRVGKVLNSVLFELKADGEQAPESVPQPAGPEALEVTLNSP | |
| 451 | FLLAVLERSSGALHFLGRVSRPLSAE | |
| Protein | MURINE GLIA DERIVED NEXIN PRECURSOR1346127: 1 . . . 397 | |
| SWISS-PROT | Name: GDN_MOUSE, Accession: Q07235 | |
| NCBI | Seq ID: 1346127 | |
| Citation | J. D. Vassalli, J. Huarte, D. Bosco, A. P. Sappino, N. | |
| | Sappino, A. Velardi, A. Wohlwend, H. Erno, D. Monard & D. | |
| | Belin (1993). Protease-nexin I as an androgen-dependent | |
| | secretory product of the murine seminal vesicle. EMBO J. | |
| | 12, 1871-1878. MEDLINE identifier: 93259128 | |
| Signal | | 1346127: 1 . . . 19 |
| Mature | GLIA DERIVED NEXIN. | 1346127: 20 . . . 397 |
| chain | | |
| Sequence | 397 aa | |
| (SEQ ID NO:33) | | |
| 1 | MNWHFPFFILTTVTLYSVHSQFNSLSLEELGSNTGIQVFNQIIKSRPHEN | |
| 51 | VVVSPHGIASILGMLQLGADGKTKKQLSTVMRYNVNGVGKVLKKINKAIV | |
| 101 | SKKNKDIVTVANAVFLRNGFKMEVPFAVRNKDVFQCEVQNVNFQDPASAS | |
| 151 | ESINFWVKNETRGMIDNLLSPNLIDGALTRLVLVNAVYFKGLWKSRFQPE | |
| 201 | STKKRTFVAGDGKSYQVPMLAQLSVFRSGSTRTPNGLWYNFIELPYHGES | |
| 251 | ISMLIALPTESSTPLSAIIPHITTKTIDSWMNTMVPKRMQLVLPKFTAVA | |
| 301 | QTDLKEPLKALGITEMFEPSKANFTKITRSESLHVSHILQKAKIEVSEDG | |
| 351 | TKASAATTAILIARSSPPWFIVDRPFLFSIRHNPTGAILFLGQVNKP | |
| Protein | HUMAN BOMAPIN 1345616: 1 . . . 397 | |
| SWISS-PROT | Name: BOMA_HUMAN, Accession: P48595 | |
| NCBI | Seq ID: 1345616 | |
| Citation | M. Riewald & R. R. Schleef (1995). Molecular cloning of | |
| | bomapin (protease inhibitor 10), a novel human serpin | |
| | that is expressed specifically in the bone marrow. J. | |
| | Biol. Chem. 270, 26754-26757. MEDLINE identifier: | |
| | 96070759 | |
| Sequence | 397 aa | |
| (SEQ ID NO:34) | | |
| 1 | MDSLATSINQFALELSKKLAESAQGKNIFFSSWSISTSLTIVYLGAKGTT | |
| 51 | AAQMAQVLQFNRDQGVKCDPESEKKRKMEFNLSNSEEIHSDFQTLISEIL | |
| 101 | KPNDDYLLKTANAIYGEKTYAFHNKYLEDMKTYFGAEPQPVNFVEASDQI | |
| 151 | RKDINSWVERQTEGKIQNLLPDDSVDSTTRMILVNALYFKGIWEHQFLVQ | |
| 201 | NTTEKPFRINETTSKPVQMMFMKKKLHIFHIEKPKAVGLQLYYKSRDLSL | |
| 251 | LILLPEDINGLEQLEKAITYEKLNEWTSADMMELYEVQLHLPKFKLEDSY | |
| 301 | DLKSTLSSMGMSDAFSQSKADFSGMSSARNLFLSNVFHKAFVEINEQGTE | |
| 351 | AAAGSGSEIDIRIRVPSIEFNANHPFLFFIRHNKTNTILFYGRLCSP | |
| Protein | PLACENTAL THROMBIN INHIBITOR HUMAN | |
| SWISS-PROT | Name: PTI6_HUMAN, Accession: P35237 | |
| NCBI | Seq ID: 464490 | |
| Cross-ref | EMBL Accession: Z22658 | |
| Citation | P. Coughlin, J. Sun, L. Cerruti, H. H. Salem & P. Bird | |
| | (1993). Cloning and molecular characterization of a human | |
| | intracellular serine proteinase inhibitor. Proc. Natl. | |
| | Acad. Sci. U.S.A. 90, 9417-9421. MEDLINE identifier: | |
| | 94022386 | |
| Sequence | 376 aa | |
| (SEQ ID NO:35) | | |
| 1 | MDVLAEANGTFALNLLKTLGKDNSKNVFFSPMSMSCALAMVYMGAKGNTA | |
| 51 | AQMAQILSFNKSGGGGDIHQGFQSLLTEVNKTGTQYLLRVANRLFGEKSC | |
| 101 | DFLSSFRDSCQKFYQAEMEELDFISAVEKSRKHINTWVAEKTEGKIAELL | |
| 151 | SPGSVDPLTRLVLVNAVYFRGNWDGQFDKENTEERLFKVSKNEEKPVQMM | |
| 201 | FKQSTFKKTYIGEIFTQILVLPYVGKELNMIIMLPDETTDLRTVEKELTY | |
| 251 | EKFVEWTRLDMMDEEEVEVSLPRFKLEESYDMESVLRNLGMTDAFELGKA | |
| 301 | DFSGMSQTDLSLSKVVHKSFVEVNEEGTEAAAATAAIMMMRCARFVPRFC | |
| 351 | ADHPFLFFIQHRKTNGILFCGRFSSP | |
| Protein | PLASMA SERINE PROTEASE INHIBITOR PRECURSOR; HUMAN | |
| SWISS-PROT | Name: IPSP_HUMAN, Accession: P05154 | |
| NCBI | Seq ID: 400068 | |
| Citation | K. Suzuki, Y. Deyashiki, J. Nishioka, K. Kurachi, M. | |
| | Akira, S. Yamamoto & S. Hashimoto (1987). Characteriza- | |
| | tion of a cDNA for human protein C inhibitor. A new | |
| | member of the plasma serine protease inhibitor super- | |
| | family. J. Biol. Chem. 262, 611-616. MEDLINE identifier: | |
| | 87109153 | |
| Signal | (experimentally determined) | 400068: 1 . . . 19 |
| Mature | PLASMA SERINE PROTEASE INHIBITOR. | 400068: 20 . . . 406 |
| chain | | |
| Sequence | 406 aa | |
| (SEQ ID NO:36) | | |
| 1 | MQLFLLLCLVLLSPQGASLHRHHPREMKKRVEDLHVGATVAPSSRRDFTF | |
| 51 | DLYRALASAAPSQNIFFSPVSISMSLAMLSLGAGSSTKMQILEGLGLNLQ | |
| 101 | KSSEKELHRGFQQLLQELNQPRDGFQLSLGNALFTDLVVDLQDTFVSAMK | |
| 151 | TLYLADTFPTNFRDSAGAMKQINDYVAKQTKGKIVDLLKNLDSNAVVIMV | |
| 201 | NYIFFKAKWETSFNHKGTQEQDFYVTSETVVRVPMMSREDQYHYLLDRNL | |
| 251 | SCRVVGVPYQGNATALFILPSEGKMQQVENGLSEKTLRKWLKMFKKRQLE | |
| 301 | LYLPKFSIEGSYQLEKVLPSLGISNVFTSHADLSGISNHSNTQVSEMVHK | |
| 351 | AVVEVDESGTRAAAATGTIFTFRSARLNSQRLVFNRPFLMFIVDNNILFL | |
| 401 | GKVNRP | |
| Protein | alpha-2-antiplasmin precursor; alpha-2-PI; alpha-2- | |
| | plasmin inhibitor precursor; Human | |
| PIR | Name: ITHUA2 | |
| NCBI | Seq ID: 2144573 | |
| Citation | S. Hirosawa, Y. Nakamura, O. Miura, Y. Sumi & N. Aoki | |
| | (1988). Organization of the human alpha 2-plasmin inhibi- | |
| | tor gene. Proc. Natl. Acad. Sci. U.S.A. 85, 6836-6840. | |
| | MEDLINE identifier: 88320531 | |
| domain | signal sequence | 2144573: 1 . . . 27 |
| domain | propeptide | 2144573: 28 . . . 39 |
| product | alpha-2-antiplasmin | 2144573: 40 . . . 491 |
| Sequence | 491 aa | |
| (SEQ ID NO:37) | | |
| 1 | MALLWGLLVLSWSCLQGPCSVFSPVSAMEPLGRQLTSGPNQEQVSPLTLL | |
| 51 | KLGNQEPGGQTALKSPPGVCSRDPTPEQTHRLARAMMAFTADLFSLVAQT | |
| 101 | STCPNLILSPLSVALALSHLALGAQNHTLQRLQQVLHAGSGPCLPHLLSR | |
| 151 | LCQDLGPGAFRLAARMYLQKGFPIKEDFLEQSEQLFGAKPVSLTGKQEDD | |
| 201 | LANINQWVKEATEGKIQEFLSGLPEDTVLLLLNAIHFQGFWRNKFDPSLT | |
| 251 | QRDSFHLDEQFTVPVEMMQARTYPLRWFLLEQPEIQVAHFPFKNNMSFVV | |
| 301 | LVPTHFEWNVSQVLANLSWDTLHPPLVWERPTKVRLPKLYLKHQMDLVAT | |
| 351 | LSQLGLQELFQAPDLRGISEQSLVVSGVQHQSTLELSEVGVEAAAATSIA | |
| 401 | MSRMSLSSFSVNRPFLFFIFEDTTGLPLFVGSVRNPNPSAPRELKEQQDS | |
| 451 | PGNKDFLQSLKGFPRGDKLFGPDLKLVPPMEEDYPQFGSPK | |
| Protein | Human maspin; protease inhibitor 5 | |
| PIR | Name: A36898 | |
| NCBI | Seq ID: 2135604 | |
| Citation | Z. Zou, A. Anisowicz, M. J. Hendrix, A. Thor, M. Neveu, | |
| | S. Sheng, K. Rafidi, E. Seftor & R. Sager (1994). Maspin, | |
| | a serpin with tumor-suppressing activity in human mammary | |
| | epithelial cells. Science 263, 526-529. MEDLINE | |
| | identifier: 94120413 | |
| Sequence | 375 aa | |
| (SEQ ID NO:38) | | |
| 1 | MDALQLANSAFAVDLFKQLCEKEPLGNVLFSPICLSTSLSLAQVGAKGDT | |
| 51 | ANEIGQVLHFENVKDIPFGFQTVTSDVNKLSSFYSLKLIKRLYVDKSLNL | |
| 101 | STEFISSTKRPYAKELETVDFKDKLEETKGQINNSIKDLTDGHFENILAD | |
| 151 | NSVNDQTKILVVNAAYFVGKWMKKFPESETKECPFRLNKTDTKPVQMMNM | |
| 201 | EATFCMGNIDSINCKIIELPFQNKHLSMFILLPKDVEDESTGLEKIEKQL | |
| 251 | NSESLSQWTNPSTMANAKVKLSIPKFKVEKMIDPKACLENLGLKHIFSED | |
| 301 | TSDFSGMSETKGVALSNVIHKVCLEITEDGGDSIEVPGARILQHKDELNA | |
| 351 | DHPFIYIIRHNKTRNIIFFGKFCSP | |
| Protein | kallikrein-binding protein precursor; contrapsin-like | |
| | protease inhibitor; growth hormone-induced proteinase | |
| | inhibitor; serine proteinase inhibitor; rat | |
| PIR | Name: B29131 | |
| NCBI | Seq ID: 92335 | |
| Citation | J. B. Yoon, H. C. Towle & S. Seelig (1987). Growth hor- | |
| | mone induces two mRNA species of the serine protease | |
| | inhibitor gene family in rat liver. J. Biol. Chem. 262, | |
| | 4284-4289. MEDLINE identifier: 87166046 | |
| Sequence | 416 aa | |
| (SEQ ID NO:39) | | |
| 1 | MAFIAALGLLMAGICPAVLCDGILGRDTLPHEDQGKGRQLHSLTLASINT | |
| 51 | DFTLSLYKKLALRNPDKNVVFSPLSISAALAILSLGAKDSTMEEILEGLK | |
| 101 | FNLTEITEEEIHQGFGHLLQRLSQPEDQAEINTGSALFIDKEQPILSEFQ | |
| 151 | EKTRALYQAEAFVADFKQCNEAKKFINDYVSNQTQGKIAELFSELDERTS | |
| 201 | MVLVNYLLFKGKWKVPFNPNDTFESEFYLDEKRSVKVPMMKIKDLTTPYI | |
| 251 | RDEELSCSVLELKYTGNASALFILPDQGKMQQVESSLQPETLKKWKDSLR | |
| 301 | PRIISELRMPKFSISTDYNLEEVLPELGIRKIFSQQADLSRITGTKNLHV | |
| 351 | SQVVHKAVLDVDETGTEGAAATAVTAALKSLPQTIPLLNFNRPFMLVITD | |
| 401 | NNGQSVFFMGKVTNPM | |
| Protein | elastase inhibitor, Leukocyte; Horse | |
| PIR | Name: A28060 | |
| NCBI | Seq ID: 89125 | |
| Citation | J. Potempa, A. Dubin, W. Watorek & J. Travis (1988). An | |
| | elastase inhibitor from equine leukocyte cytosol belongs | |
| | to the serpin superfamily. Further characterization and | |
| | amino acid sequence of the reactive center. J. Biol. | |
| | Chem. 263, 7364-7369. MEDLINE identifier: 88213423 | |
| Sequence | 18 aa | |
| (SEQ ID NO:40) | | |
| 1 | LAMLMPEENF NADHPFIF | |
| Protein | PEDF; secreted glycoprotein; neurotrophic region, | |
| | homologous serpin reactive site. | |
| NCBI | Seq ID: 1655809 | |
| Citation | L. Perez-Mediavilla, C. Chew, P. Campochiaro, D. J. | |
| | Zack & S. P. Becerra. Expression of bovine PEDF. Unpub- | |
| | lished Coding region function: neurotrophic factor, | |
| | 1655808: 12 . . . 1262 serpin. | |
| Sequence | 416 aa | |
| (SEQ ID NO:41) | | |
| 1 | MQALVLLLWTGALLGFGRCQNAGQEAGSLTPESTGAPVEEEDPFFKVPVN | |
| 51 | KLAAAVSNFGYDLYRVRSGESPTANVLLSPLSVATALSALSLGAEQRTES | |
| 101 | NIHRALYYDLISNPDIHGTYKDLLASVTAPQKNLKSASRIIFERKLRIKA | |
| 151 | SFIPPLEKSYGTRPRILTGNSRVDLQEINNWVQAQMKGKVARSTREMPSE | |
| 201 | ISIFLLGVAYFKGQWVTKFDSRKTSLEDFYLDEERTVKVPMMSDPQAVLR | |
| 251 | YGLDSDLNCKIAQLPLTGSTSIIFFLPQKVTQNLTLIEESLTSEFIHDID | |
| 301 | RELKTVQAVLTIPKLKLSYEGELTKSVQELKLQSLFDAPDFSKITGKPIK | |
| 351 | LTQVEHRVGFEWNEDGAGTNSSPGVQPARLTFPLDYHLNQPFIFVLRDTD | |
| 401 | TGALLFIGKILDPRGT | |
| Protein | PIGMENT EPITHELIUM-DERIVED FACTOR PRECURSOR; HUMAN | |
| SWISS-PROT | Name: PEDF_HUMAN, Accession: P36955 | |
| NCBI | Seq ID: 1352735 | |
| Citation | R. J. Pignolo, V. J. Cristofalo & M. O. Rotenberg (1993). | |
| | Senescent WI-38 cells fail to express EPC-1, a gene | |
| | induced in young cells upon entry into the GO state. J. | |
| | Biol. Chem. 268, 8949-8957. MEDLINE identifier: 93232057 | |
| Signal | | 1352735: 1 . . . 17 |
| Mature | PIGMENT EPITHELIUM-DERIVED FACTOR. | 1352735: 18 . . . 418 |
| chain | | |
| Sequence | 418 aa | |
| (SEQ ID NO:42) | | |
| 1 | MQALVLLLCIGALLGHSSCQNPASPPEEGSPDPDSTGALVEEEDPFFKVP | |
| 51 | VNKLAAAVSNFGYDLYRVRSSMSPTTNVLLSPLSVATALSALSLGADERT | |
| 101 | ESIIHRALYYDLISSPDIHGTYKELLDTVTAPQKNLKSASRIVFEKKLRI | |
| 151 | KSSFVAPLEKSYGTRPRVLTGNPRLDLQEINNWVQAQMKGKLARSTKEIP | |
| 201 | DEISILLLGVAHFKGQWVTKFDSRKTSLEDFYLDEERTVRVPMMSDPKAV | |
| 251 | LRYGLDSDLSCKIAQLPLTGSMSIIFFLPLKVTQNLTLIEESLTSEFIHD | |
| 301 | IDRELKTVQAVLTVPKLKLSYEGEVTKSLQEMKLQSLFDSPDFSKITGKP | |
| 351 | IKLTQVEHRAGFEWNEDGAGTTPSPGLQPAHLTFPLDYHLNQPFIFVLRD | |
| 401 | TDTGALLFIGKILDPRGP | |
| Protein | PLASMINOGEN ACTIVATOR INHIBITOR-2, PLACENTAL; HUMAN | |
| SWISS-PROT | Name: PAI2_HUMAN, Accession: P05120 | |
| NCBI | Seq ID: 1352712 | |
| Cross-ref | GenBank Accession: J02685 | |
| Citation | R. D. Ye, T. C. Wun & J. E. Sadler (1987). cDNA cloning | |
| | and expression in Escherichia coli of a plasminogen | |
| | activator inhibitor from human placenta. J. Biol. Chem; | |
| | 262, 3718-3725. MEDLINE identifier: 87137674 | |
| Sequence | 415 aa | |
| (SEQ ID NO:43) | | |
| 1 | MEDLCVANTLFALNLFKHLAKASPTQNLFLSPWSISSTMAMVYMGSRGST | |
| 51 | EDQMAKVLQFNEVGANAVTPMTPENFTSCGFMQQIQKGSYPDAILQAQAA | |
| 101 | DKIHSSFRSLSSAINASTGNYLLESVNKLFGEKSASFREEYIRLCQKYYS | |
| 151 | SEPQAVDFLECAEEARKKINSWVKTQTKGKIPNLLPEGSVDGDTRMVLVN | |
| 201 | AVYFKGKWKTPFEKKLNGLYPFRVNSAQRTPVQMMYLREKLNIGYIEDLK | |
| 251 | AQILELPYAGDVSMFLLLPDEIADVSTGLELLESEITYDKLNKWTSKDKM | |
| 301 | AEDEVEVYIPQFKLEEHYELRSILRSMGMEDAFNKGRANFSGMSERNDLF | |
| 351 | LSEVFHQAMVDVNEEGTEAAAGTGGVMTGRTGHGGPQFVADHPFLFLIMH | |
| 401 | KITNCILFFGRFSSP | |
| Protein | THYROXINE-BINDING GLOBULIN PRECURSOR; HUMAN | |
| SWISS-PROT | Name: THBG_HUMAN, Accession: P05543 | |
| NCBI | Seq ID: 1351236 | |
| Cross-ref | NCBI Seq ID: 338697 | |
| Cross-ref | GenBank Accession: L13470 | |
| Citation | I. L. Flink, T. J. Bailey, T. A. Gustafson, B. E. | |
| | Markham & E. Morkin (1986). Complete amino acid sequence | |
| | of human thyroxine-binding globulin deduced from cloned | |
| | DNA: close homology to the serine antiproteases. Proc. | |
| | Natl. Acad. Sci. U.S.A. 83, 7708-7712. MEDLINE | |
| | identifier: 87017018 | |
| Signal | (experimentally determined) | 1351236: 1 . . . 20 |
| Mature | THYROXINE-BINDING GLOBULIN. | 1351236: 21 . . . 415 |
| chain | (experimentally determined) | |
| Sequence | 415 aa | |
| (SEQ ID NO:44) | | |
| 1 | MSPFLYLVLLVLGLHATIHCASPEGKVTACHSSQPNATLYKMSSINADFA | |
| 51 | FNLYRRFTVETPDKNIFFSPVSISAALVMLSFGACCSTQTEIVETLGFNL | |
| 101 | TDTPMVEIQHGFQHLICSLNFPKKELELQIGNALFIGKHLKPLAKFLNDV | |
| 151 | KTLYETEVFSTDFSNISAAKQEINSHVEMQTKGKVVGLIQDLKPNTIMVL | |
| 201 | VNYIHFKAQWANPFDPSKTEDSSSFLIDKTTTVQVPMMHQMEQYYHLVDM | |
| 251 | ELNCTVLQMDYSKNALALFVLPKEGQMESVEAAMSSKTLKKWNRLLQKGW | |
| 301 | VDLFVPKFSISATYDLGATLLKMGIQHAYSENADFSGLTEDNGLKLSNAA | |
| 351 | HKAVLHIGEKGTEAAAVPEVELSDQPENTFLHPIIQIDRSFMLLILERST | |
| 401 | RSILFLGKVVNPTEA | |
| Protein | HUMAN HEPARIN COFACTOR II PRECURSOR | |
| SWISS-PROT | Name: HEP2_RABIT, Accession: P47776 | |
| NCBI | Seq ID: 1346272 | |
| Cross-ref | NCBI Seq ID: 688191 | |
| Citation | W. P. Sheffield, P. D. Schuyler & M. A. Blajchman (1994). | |
| | Molecular cloning and expression of rabbit heparin co- | |
| | factor II: a plasma thrombin inhibitor highly conserved | |
| | between species. Thromb. Haemost. 71, 778-782. MEDLINE | |
| | identifier: 95064663 | |
| Signal | | 1346272: 1 . . . 19 |
| Mature | HEPARIN COFACTOR II. | 1346272: 20 . . . 480 |
| chain | | |
| Sequence | 480 aa | |
| (SEQ ID NO:45) | | |
| 1 | MQHRPHLLLISLTIMSVCGGSNGLTDQLNNKNLTMPLLPIEFHKENTVTN | |
| 51 | DWIPEGEEDDDYLDLEKLLSEDDDYIDIIDAVSPTDSEASAGNILQLFQG | |
| 101 | KSRIQRLNILNAKFAFSLYRALKDQANAFDNIFIAPVGISTAMGMISLGL | |
| 151 | KGETHEQVHSVLHFRDFVNASSKYEILTIHNLFRKLTHRLFRRNFGYTLR | |
| 201 | SVNDLYVQKQFPIREDFKAKVREYYFAEAQAADFSDPAFISKANNHILKV | |
| 251 | TKGLIKEALENVDPATQMMILNCIYFKGTWVNKFPVEMTHNHNFRLNERE | |
| 301 | VVKVSMMQTKGNFLAANDQELACDVLQLEYVGGISMLIVVPHKLSGMKTL | |
| 351 | EAQLTPQVVERWQKSMTNRTREVLLPKFKLEKNYNLVEALKSMGVTELFD | |
| 401 | KNGNMSGISDQGITMDLFKHQGTITVNEEGTQAAAVTTVGFMPLSTQVRF | |
| 451 | TVDRPFLFLVYEHRTSCLLFMGKVANPVRS | |
| Protein | HEPARIN COFACTOR II PRECURSOR; HUMAN | |
| SWISS-PROT | Name: HEP2_HUMAN, Accession: P05546 | |
| NCBI | Seq ID: 123055 | |
| Cross-ref | NCBI Seq ID: 183910 | |
| Citation | R. Herzog, S. Lutz, N. Bun, J. C. Marasa, M. A. Blinder & | |
| | D. M. Tollefsen (1991). Complete nucleotide sequence of | |
| | the gene for human heparin cofactor II and mapping to | |
| | chromosomal band 22q11. Biochemistry 30, 1350-1357. MED- | |
| | LINE identifier: 91120782 | |
| Signal | (experimentally determined) | 123055: 1 . . . 19 |
| Mature | HEPARIN COFACTOR II. | 123055: 20 . . . 499 |
| chain | | |
| Sequence | 499 aa | |
| (SEQ ID NO:46) | | |
| 1 | MKHSLNALLIFLIITSAWGGSKGPLDQLEKGGETAQSADPQWEQLNNKNL | |
| 51 | SMPLLPADFHKENTVTNDWIPEGEEDDDYLDLEKIFSEDDDYIDIVDSLS | |
| 101 | VSPTDSDVSAGNILQLFHGKSRIQRLNILNAKFAFNLYRVLKDQVNTFDN | |
| 151 | IFIAPVGISTAMGMISLGLKGETHEQVHSILHFKDFVNASSKYEITTIHN | |
| 201 | LFRKLTHRLFRRNFGYTLRSVNDLYIQKQFPILLDFKTKVREYYFAEAQI | |
| 251 | ADFSDPAFISKTNNHIMKLTKGLIKDALENIDPATQMMILNCIYFKGSWV | |
| 301 | NKFPVEMTHNHNFRLNEREVVKVSMMQTKGNFLAANDQELDCDILQLEYV | |
| 351 | GGISMLIVVPHKMSGMKTLEAQLTPRVVERWQKSMTNRTREVLLPKFKLE | |
| 401 | KNYNLVESLKLMGIRMLFDKNGNMAGISDQRIAIDLFKHQGTITVNEEGT | |
| 451 | QATTVTTVGFMPLSTQVRFTVDRPFLFLIYEHRTSCLLFMGRVANPSRS | |
| Protein | HUMAN ANTITHROMBIN-III PRECURSOR | |
| SWISS-PROT | Name: ANT3_HUMAN, Accession: P01008 | |
| NCBI | Seq ID: 113936 | |
| Cross-ref | GenBank Accession: M21642 | |
| Citation | S. C. Bock, K. L. Wion, G. A. Vehar & R. M. Lawn (1982). | |
| | Cloning and expression of the cDNA for human antithrombin | |
| | III. Nucleic Acids Res. 10, 8113-8125. MEDLINE identi- | |
| | fier: 83143280 | |
| Signal | (experimentally determined) | 113936: 1 . . . 32 |
| Mature | ANTITHROMBIN-III. | 113936: 33 . . . 464 |
| chain | | |
| Sequence | 464 aa | |

| (SEQ ID NO:47) | |
|---|---|
| 1 | MYSNVIGTVTSGKRKVYLLSLLLIGFWDCVTCHGSPVDICTAKPRDIPMN |
| 51 | PMCIYRSPEKKATEDEGSEQKIPEATNRRVWELSKANSRFATTFYQHLAD |
| 101 | SKNDNDNIFLSPLSISTAFAMTKLGACNDTLQQLMEVFKFDTISEKTSDQ |
| 151 | IHFFFAKLNCRLYRKANKSSKLVSAKRLFGDKSLTFNETYQDISELVYGA |
| 201 | KLQPLDFKENAEQSRAAINKWVSNKTEGRITDVIPSEAINELTVLVLVNT |
| 251 | IYFKGLWKSKFSPENTRKELFYKADGESCSASMMYQEGKFRYRRVAEGTQ |
| 301 | VLELPFKGDDITMVLILPKPEKSLAKVEKELTPEVLQEWLDELEEMMLVV |
| 351 | HMPRFRIEDGFSLKEQLQDMGLVDLFSPEKSKLPGIVAEGRDDLYVSDAF |
| 401 | HKAFLEVNEEGSEAAASTAVVIAGRSLNPNRVTFKANRPFLVFIREVPLN |
| 451 | TIIFMGRVANPCVK |
| Protein | human pigment epithelium-derived factor |
| NCBI | Seq ID: 1144299 |
| Citation | J Tombran-Tink, K Mazuruk, I. Rodriguez, R. E. Kouri, D. |
| | Chung, T. Linker & G. J. Chader. Cloning and molecular |
| | characterization of the human gene for the neurotrophic |
| | serpin PEDF: conservation, polymorphism and hereditary |
| | studies. |
| Sequence | 362 aa |
| (SEQ ID NO:48) | |
| 1 | MQALVLLLCIGALLGHSSCQNPASPPEEGSPDPDSTGALVEEEDPFFKVP |
| 51 | VNKLAAAVSNFGYDLYRVRSSMSPTTNVLLSPLSVATALSALSLGAEQRT |
| 101 | ESIIHRALYYDLISSPDIHGTYKELLDTVTAPQKNLKSASRIVFEKKLRI |
| 151 | KSSFVAPLEKSYGTRPRVLTGNPRLDLQEINNWVQAQMKGKLARSTKEIP |
| 201 | DEISILLLGVAHFKGQWVTKFDSRKTSLEDFYLDEERTVRVPMMSDPKAV |
| 251 | LRYGLDSDLSCKIAQLPLTGSMSIIFFLPLKVTQNLTLIEESLTSEFIHD |
| 301 | IDRELKTVQAVLTVPKLKLSYEGEVTKSLQEMKLQSLFDSPDFSKITGKP |
| 351 | IKLTQGGTPGWL |
| Protein | Name: serine protease inhibitor |
| NCBI | Seq ID: 439153 |
| Citation | C Huang, M Lee, F Huang & G Chang (1995). A protease |
| | inhibitor of the serpin family is a major protein in carp |
| | perimeningeal fluid: II. CDNA cloning, sequence analysis, |
| | and Escherichia coli expression. J. Neurochem. 64, 1721- |
| | 1727. MEDLINE identifier: 95198028 Coding region |
| | function: protease inhibitor. 439152: 34 . . . 1266 |
| Sequence | 410 aa |
| (SEQ ID NO:49) | |
| 1 | MAWAAPHEGHDHDGHPADHYHHLHHGKDEAHPSHSGEDACHLLSPHNADF |
| 51 | AFSLYKKLALHPDAQGKNIFFSPVGISMALSMLAVGAKGSTLSQIYSSLG |
| 101 | YSGLKAQQVNEGYEHLIHMLGHSQDTMQLEAGAGVAIREGFKVVDQFLKD |
| 151 | VQHYYNSEAFSVDFSKPEIAAEEINQFIAKKTNDKITDMVKDLDSDMVMM |
| 201 | LINYMYFRGKWDKPFEAQLTHKAEFKVDKDTTVQVDMMKRTGRYDIYQDP |
| 251 | VNQTTVMMVPYKGNTSMMIVLPDEGKMKDVEESICRHHLKNWHDKLFRSS |
| 301 | VDLFMPKFSISATSKLNDILTEMGVTDAFSDTADFSGMTEELKVKVSQVV |
| 351 | HKAVLSVDEKGTEAAAATTIEIMPMSLPGTVMLNRPFLVLIVEDTTKSIL |
| 401 | FMGKITNPTV |
| Protein | Carp alpha-1 antitrypsin |
| NCBI | Seq ID: 213046 |
| Citation | C Huang, M Lee, F Ruang & G Chang (1995). A protease |
| | inhibitor of the serpin family is a major protein in carp |
| | perimeningeal fluid: II. CDNA cloning, sequence analysis, |
| | and Escherichia coli expression. J. Neurochem. 64, 1721- |
| | 1727. MEDLINE identifier: 95198028 |
| Sequence | 372 aa |
| (SEQ ID NO:50) | |
| 1 | MPATCLLHTMLTLPSPSTRNLRSIQMPRARTFSSPSRYRNGFEHAGCRCQ |
| 51 | GSTLSQIYSSLGYSGLQASQVNEGYEHLIHMLGHSREAMQLEAGAGVAIR |
| 101 | EGFKVVDQFLKDVQHYYNSEAFSVDFSKPEIAAEEINQFIAKKTNDKITN |
| 151 | MVKDLDSDTVMMLINYMYFRGKWDKPFDAQLTHKADFKVDEDTTVQVDMM |
| 201 | KRTGRYDIYQDPVNQTTVMMVPYKGNTSMMIIFPDDGKMKELEESISRHH |
| 251 | LKNWHDKLFRSSVDLFMPKFSITATSKLKGILEDMGVTDAFGDTADLSGL |
| 301 | TEEVKVKVSQVVHKAVLSVDEKGTEAAAATTIEIMPMSLPDTVILNRPFL |
| 351 | VLIVEDTTKSILFMGKITNPTE |

### 6. REFERENCES

The following references, to the extent that they provide exemplary procedural or other details supplementary to those set forth herein, are specifically incorporated herein by reference in whole or in part:
U.S. Pat. No. 4,216,209.
U.S. Pat. No. 4,683,195.
U.S. Pat. No. 4,683,202.
U.S. Pat. No. 4,800,159.
U.S. Pat. No. 4,883,750.
U.S. Pat. No. 4,987,071.
U.S. Pat. No. 5,037,746.
U.S. Pat. No. 5,093,246.
U.S. Pat. No. 5,098,887.
U.S. Pat. No. 5,116,742.
U.S. Pat. No. 5,145,684.
U.S. Pat. No. 5,219,727.
U.S. Pat. No. 5,238,921.
U.S. Pat. No. 5,297,721.
U.S. Pat. No. 5,334,711.
U.S. Pat. No. 5,348,978.
U.S. Pat. No. 5,354,855.
U.S. Pat. No. 5,399,346.
U.S. Pat. No. 5,399,363.
U.S. Pat. No. 5,449,661.
U.S. Pat. No. 5,455,166.
U.S. Pat. No. 5,466,468.
U.S. Pat. No. 5,543,158.
U.S. Pat. No. 5,552,157.
U.S. Pat. No. 5,552,397.
U.S. Pat. No. 5,565,213.
U.S. Pat. No. 5,567,434.
U.S. Pat. No. 5,631,359.
U.S. Pat. No. 5,639,655.
U.S. Pat. No. 5,641,515.
U.S. Pat. No. 5,646,020.
U.S. Pat. No. 5,646,031.
U.S. Pat. No. 5,648,211.
U.S. Pat. No. 5,712,124.
U.S. Pat. No. 5,738,868.
U.S. Pat. No. 5,741,516.
U.S. Pat. No. 5,744,311.
U.S. Pat. No. 5,795,587.
U.S. Pat. No. 5,863,736.
Int. Pat. Appl. No. PCT/US87/00880.
Int. Pat. Appl. No. PCT/US88/10315.
Int. Pat. Appl. No. PCT/US89/01025.
Int. Pat. Appl. Publ. No. WO89/06700.
Int. Pat. Appl. Publ. No. WO90/07641.
Int. Pat. Appl. Publ. No. WO91/03162.
Int. Pat. Appl. Publ. No. WO92/07065.
Int. Pat. Appl. Publ. No. WO93/15187.
Int. Pat. Appl. Publ. No. WO93/23569.
Int. Pat. Appl. Publ. No. WO94/02595.
Int. Pat. Appl. Publ. No. WO94/13688.
Eur. Pat. Appl. Publ. No. EP0329822.
Eur. Pat. Appl. Publ. No. EP0360257.
Eur. Pat. Appl. Publ. No. EP320308.
Eur. Pat. Appl. Publ. No. EP92110298.4.
Eur. Pat. Appl. Publ. No. EP0273085.
Great Britain Pat. Appl. No. 2202328.
Afione, Conrad, Kearns, Chunduru, Adams, Reynolds, Guggino, Cutting, Carter and Flotte, “In vivo model of adeno-associated virus vector persistence and rescue,” J. Virology, 70:3235-41, 1996.
Alejandro, Lehmann, Ricordi, Kenyon, Angelico, Burke, Esquenazi, Nery, Betancourt, Kong, Miller and Mintz, “Long-term function (6 years) of islet allografts in type 1 Type I diabetes,” Diabetes, 46:1983-89, 1997.
Arreaza, Cameron, Jaramillo, Gill, Hardy, Laupland, Rapoport, Zucker, Chakrabarti, Chensue, Qin, Singh and Delovitch, “Neonatal activation of CD28 signaling overcomes T cell energy and prevents autoimmune diabetes by an IL-4-dependent mechanism,” J. Clin. Invest., 100:2243-53, 1997.
Atkinson and Leiter, “The NOD mouse model of type 1 Type I diabetes: as good as it gets?,” Nat. Med., 5:601-04, 1999.
Atkinson and Maclaren, “The pathogenesis of insulin-dependent diabetes mellitus,” N. Engl. J. Med., 331:1428-36, 1994.
Bach, “Insulin-dependent diabetes mellitus as an autoimmune disease,” Endocr. Rev., 15:516-42, 1994.
Bach, “Insulin dependent diabetes mellitus as a beta-cell targeted disease of immunoregulation,” J. Autoimmun., 8:439-463, 1995.
Bach and Chatenoud, “Tolerance to islet autoantigens in Type 1 diabetes,” Annu. Rev. Immunol., 19:131-61, 2001.
Balasa and Sarvetnick, “The paradoxical effects of interleukin 10 in the immunoregulation of autoimmune diabetes,” J. Autoimmun., 9:283-86, 1996.
Balasa, La Cava, Van Gunst, Mocnik, Balakrishna, Nguyen, Tucker and Sarvetnick, “A mechanism for IL-10-mediated diabetes in the nonobese diabetic (NOD) mouse: ICAM-1 deficiency blocks accelerated diabetes,” J. Immunol., 165:7330-37, 2000a.
Balasa, Van Gunst, Jung, Balakrishna, Santamaria, Hanafusa, Itoh and Sarvetnick, “Islet-specific expression of IL-10 promotes diabetes in nonobese diabetic mice independent of Fas, perforin, TNF receptor-1, and TNF receptor-2 molecules,” J. Immunol., 165:2841-47, 2000b.
Bendelac, Carnaud, Boitard and Bach, “Syngeneic transfer of autoimmune diabetes from diabetic NOD mice to healthy neonates. Requirement for both L3T4+ and Lyt-2+T cells,” J. Exp. Med., 166:823-32, 1987.
Benhamou, Mullen, Sshaaked, Bahmiller and Csete, “Decreased alloreactivity to human islets secreting recombinant viral interleukin 10,” Transplantation, 62:1306-12, 1996.
Berns, in FIELDS VIROLOGY, (Fields, (ed.), Raven Press, Philadelphia, Pa., pp. 2173-97, 1996.
Borriello and Krauter, “Multiple murine alpha 1-protease inhibitor genes show unusual evolutionary divergence,” Proc. Nat'l Acad. Sci. USA, 88:9417-21, 1991.
Boskovic and Twining, “Local control of α1-proteinase inhibitor levels: regulation of α1-proteinase inhibitor in the human cornea by growth factors and cytokines,” Biochim. Biophys. Acta, 1403:37-46, 1998.
Bottino, Fernandez, Ricordi, Lehmann, Tsan, Oliver and Inverardi, “Transplantation of allogenic islets of Langerhans in the rat liver: effects of macrophage depletion on graft survival and microenvironment activation,” Diabetes, 47:316-23, 1998.
Bowman, Campbell, Darrow, Ellis, Suresh and Atkinson, “Immunological and metabolic effects of prophylactic insulin therapy in the NOD-scid/scid adoptive transfer model of IDDM,” Diabetes, 45:205-08, 1996.
Brantly, M. L., Wittes, J. T., Vogelmeier, C. F., Hubbard, R. C., Fells, G. A., Crystal, R. G. (1991) Chest 100:703-708.
Brass, Crawford, Narciso and Gollan, “Evaluation of University of Wisconsin cold-storage solution in warm hypoxic perfusion of rat liver: the addition of fructose reduces injury,” Gastroenterology, 105:1455-63, 1993.
Briggs, M. R., Kadonga, J. T., Bell, S. P., Tjian, R. (1986) Science 234:47-52.
Cameron, Areaza, Zucker, Chensue, Strieter, Chaaakrabaarti and Delovitch, “IL-4 prevents insulitis and insulin-dependent diabetes mellitus in nonobese diabetic mice by potentiation of regulatory T helper-2 cell function,” J. Immunol., 159:4686-92, 1997.
Cameron, Strathdee, Holmes, Arreaza, Dekaban and Delovitch, “Biolistic-mediated interleukin 4 gene transfer prevents the onset of type 1Type I diabetes,” Hum. Gene Ther., 11: 1647-56, 2000.
Carrell et al., “Structure and variation of human alpha 1-antitrypsin,” Nature, 298:329-34, 1982.
Carter et al., Virology, 126:505-15, 1983.
Carter, Mendelson and Trempe, Handbook of Parvoviruses, CRC Press, Boca Raton, pp. 169-226, 1990.
Chang and Prud'homme, “Intramuscular administration of expression plasmids encoding interferon-gamma receptor/IgG1 or IL-4/IgG1 chimeric proteins protects from autoimmunity,” J. Gene Med., 1:415-23, 1999.
Chao, Liu, Rabinowitz, Li, Samulski and Walsh, “Several log increase in therapeutic transgene delivery by distinct adeno-associated viral serotype vectors,” Mol. Ther., 2:619-23, 2000.
Churg, Dai, Zay, Karsan, Hendricks, Yee, Martin, MacKenzie, Xie, Zhang, Shapiro and Wright, “α-1-antitrypsin and a broad spectrum metalloprotease inhibitor, RS113456, have similar acute anti-inflammatory effects,” Lab. Invest., 81:1119-31, 2001.
Clark, Sferra and Johnson, “Recombinant adeno-associated viral vectors mediate long-term transgene expression in muscle,” Hum. Gene Ther., 8:659-69, 1997.
Cowan, Baron, Crack, Coulber, Wilson and Rabinovitch, “Elafin, a serine elastase inhibitor, attenuates post-cardiac transplant coronary arteriopathy and reduces myocardial necrosis in rabbits after heterotopic cardiac transplantation,” J. Clin. Invest., 97:2452-68, 1996.
Cretin, Buhler, Fournier, Caulfield, Oberholzer, Mentha and Morel, “Human islet allotransplantation: world experience and current status,” Dig. Surg., 15:656-62, 1998.
Cukor, Blacklow, Hoggan and Berns, in THE PARVOVIRUSES, (Berns (ed.), Plenum Press, New York, N.Y., pp. 33-66, 1983.
Davies, Mueller, Minson, Oconner, Krahl and Sarvetnick, “Interleukin-4 secretion by the allograft fails to affect the allograft-specific interleukin-4 response in vitro,” Transplantation, 67:1583-89, 1999.
Delovitch and Singh, “The nonobese diabetic mouse as a model of autoimmune diabetes: immune dysregulation gets the NOD,” Immunity, 7:727-38, 1997.
Deng, Ketchum, Yang, Kucher, Weber, Shaked, Naji and Brayman, “IL-10 and TGF-β gene transfer to rodent islets: effect on xenogeneic islet graft survival in naive and B-cell-deficient mice,” Trans. Proc., 29:2207-68, 1997.
Deshpande, Chopra, Rangarajan, Shashidhara, Rodrigues and Krishna, J. Biol. Chem., 272:10664-68, 1997.
Dhami, Gilks, Xie, Zay, Wright and Churg, “Acute cigarette smoke-induced connective tissue breakdown is mediated by neutrophils and prevented by α1-antitrypsin,” Am. J. Respir. Cell Mol. Biol., 22:244-52, 2000.
Ding, Qin, Kotenko, Pestka and Bromberg, “A single amino acid determines the immunostimulatory activity of interleukin 10,” J. Exp. Med., 191:213-23, 2000.
Duan, Yue, Yan and Engelhardt, “A new dual-vector approach to enhance recombinant adeno-associated virus-mediated gene expression through intermolecular cis activation,” Nat. Med., 6:595-98, 2000.
Dunn, “Problems related to immunosuppression. Infection and malignancy occurring after solid organ transplantation,” Crit. Care Clin., 6:955-77, 1990.
Fellowes, Etheridge, Coade, Cooper, Stewart, Miller and Woo, “Amelioration of established collagen induced arthritis by systemic IL-10 gene delivery,” Gene Ther., 7:967-77, 2000.
Ferrari, Samulski, Shenk and Samulski, “Second strand synthesis is a rate-limiting step for efficient transduction by recombinant adeno-associated virus vectors,” J. Virol., 70:3227-34, 1996.
Ferrari, Xiao, McCarty and Samulski, Nature Med., 3:1295-97, 1997.
Fischer et al., “Induction of alpha1-antitrypsin synthesis in human articular chondrocytes by interleukin-6-type cytokines: evidence for a local acute-phase response in the joint,” Arthritis Rheum., 42:1936-45, 1999.
Fisher, Gao, Weitzman, DeMatteo, Burda and Wilson, “Transduction with recombinant adeno-associated virus for gene therapy is limited by leading-strand synthesis,” J. Virol., 70:520-32, 1996.
Fisher, Jooss, Alston, Yang, Haecker, High, Pathak, Raper and Wilson, “Recombinant adeno-associated virus for muscle directed gene therapy,” Nat. Med., 3:306-12, 1997.
Flannery, Zolotukhin, Vaquero, LaVail, Muzyczka and Hauswirth, “Efficient photoreceptor-targeted gene expression in vivo by recombinant adeno-associated virus,” Proc. Natl. Acad. Sci. USA, 94:6916-21, 1997.
Flotte and Carter, “Adeno-associated virus vectors for gene therapy,” Gene Ther., 2:357-62, 1995.
Flotte and Ferkol, “Genetic therapy. Past, present, and future,” Pediatr. Clin. North Am., 44:153-78, 1997.
Flotte, Afione, Conrad, McGrath, Solow, Oka, Zeitlin, Guggino and Carter, “Stable in vivo expression of the cystic fibrosis transmembrane conductance regulator with an adeno-associated virus vector,” Proc. Natl. Acad. Sci. USA, 90:10613-67, 1993.
Flotte, Agarwal, Wang, Song, Fenjves, Inverardi, Chestnut, Afione, Loiler, Wasserfall, Kapturczak, Ellis, Nick and Atkinson, “Efficient ex vivo transduction of pancreatic islet cells with recombinant adeno-associated virus vectors,” Diabetes, 50:515-20, 2001.
Fujita, Yui, Kusumota, Serizawa, Makino and Tochino, “Lymphocytic insulitis in a nonobese diabetic (NOD) strain of mice: an immunohistochemical and electron microscope investigation,” Biomed. Res., 3:429, 1982.
Gallichan, Balasa, Davies and Sarvetnick, “Pancreatic IL-4 expression results in islet-reactive Th2 cells that inhibit diabetogenic lymphocytes in the nonobese diabetic mouse,” J. Immunol., 1163:1696-703, 1999.
Gallichan, Kafri, Krahl, Verma and Sarvetnick, “Lentivirus-mediated transduction of islet grafts with interleukin 4 results in sustained gene expression and protection from insulitis,” Hum. Gene Ther., 9:2717-26, 1998.
Garver, Jr., Chytil, Courtney and Crystal, Science, 237:762-64, 1987.
Geboes, Ray, Rutgeerts, Callea, Desmet and Vantrappen, “Morphological identification of α-I-antitrypsin in the human small intestine,” Histopathology, 6:55-60, 1982.
Giannoukakis, Rundert, Robbins and Trucco, “Targeting autoimmune diabetes with gene therapy, Diabetes, 48:2107-21, 1999.
Goudy, Song, Wasserfall, Zhang, Kapturczak, Muir, Powers, Scott-Jorgensen, Campbell-Thompson, Crawford, Ellis, Flotte and Atkinson, “Adeno-associated virus vector-mediated IL-10 gene delivery prevents Type 1Type I diabetes in NOD mice,” Proc. Natl. Acad. Sci. USA, 98:13913-18, 2001.
Goudy et al., “Elucidation of time and dose dependencies using AAV-IL-10 gene therapy for prevention of type 1 diabetes in the NOD mouse,” Mol. Ther., 5:S17 (Abstr. 46), 2002.
Graser, DiLorenzo, Wang, Christianson, Chapman, Roopenian, Nathenson and Serreze, “Identification of a CD8 T cell that can independently mediate autoimmune diabetes development in the complete absence of CD4 T cell helper functions,” J. Immunol, 164:3913-18, 2000.
Guo, Chong, Shen, Foster, Sankary, McChesney, Mital, Jensik, Gebel and Williams, “In vivo effects of leflunomide on normal pancreatic islet and syngeneic islet graft function,” Transplantation, 63:716-21, 1997.
Hahn, Laube, Lucke, Kloting, Kohnert and Warzock, “Toxic effects of cyclosporine on the endocrine pancreas of Wistar rats,” Transplantation, 41:44-47, 1986.
Haskins, Portas, Bradley, Wegmann and Lafferty, “T-lymphocyte clone specific for pancreatic islet antigen,” Diabetes, 37:1444-48, 1988.
Hauswirth, Lewin, Zolotukhin and Muzyczka, “Production and purification of recombinant adeno-associated virus,” Methods Enzymol., 316:743-61, 2000.
Hering, Browatzki, Schultz, Bretzel and Federlin, “Clinical islet transplantation—registry report, accomplishments in the past and future research needs,” Cell Transplant, 2:269-82, discussion 283-305, 1993.
Hermonat, Labow, Wright, Berns and Muzyczka, “Genetics of adeno-associated virus: isolation and preliminary characterization of adeno-associated virus type 2 mutants,” J. Virol., 51:329-39, 1984.
Hirano, Fujihira, Ohara, Katsuki and Noguchi, “Morphological and functional changes of islets of Langerhans in FK506-treated rats,” Transplantation, 53:889-94, 1992.
Janciauskiene, “Conformational properties of serine proteinase inhibitors (serpins) confer multiple pathophysiological roles,” Biochim. Biophys. Acta, 1535:221-35, 2001.
Jindal, “Post-transplant diabetes mellitus—a review,” Transplantation, 58:1289-98, 1994.
Johansson et al., “Alpha-1-antitrypsin is present in the specific granules of human eosinophilic granulocytes,” Clin. Exp. Allergy, 31:379-86, 2001.
Jooss, Yang, Fisher and Wilson, “Transduction of dendritic cells by DNA viral vectors directs the immune response to transgene products in muscle fibers,” J. Virol., 72:4212-23, 1998.
Joslin et al., “The SEC receptor recognizes a pentapeptide neodomain of alpha 1-antitrypsin-protease complexes,” J. Biol. Chem., 266:11282-88, 1991.
Kaplitt, Leone, Samulski, Xiao, Pfaff, O'Malley and During, “Long-term gene expression and phenotypic correction using adeno-associated virus vectors in the mammalian brain,” Nat. Genet., 8:148-54, 1994.
Kapturczak, Flotte and Atkinson, Curr. Mol. Med., 1:245-58, 2001.
Kaufman, Platt, Rabe, Dunn, Bach and Sutherland, “Differential roles of Mac-1+ cells, and CD4+ and CD8+ T lymphocytes in primary nonfunction and classic rejection of islet allografts,” J. Exp. Med., 172:291-302, 1990.
Kearns, Afione, Fulmer, Caruso, Flotte and Cutting, “Recombinant adeno-associated virus (AAV-CFTR) vectors,” Gene Therapy, 3:748-755, 1996.
Kenyon, Alejandro, Mintz and Ricordi, “Islet cell transplantation: beyond the paradigms,” Diabetes Metab. Rev., 12:361-72, 1996.
Keppler, Markert, Carnal, Berdoz, Bamat and Sordat, “Human colon carcinoma cells synthesize and secrete α1-proteinase inhibitor,” Biol. Chem. Hoppe-Seyler, 377:301-11, 1996.
Kessler, Podsakoff, Chen, McQuiston, Colosi, Matelis, Kurtzman and Byrne, “Gene delivery to skeletal muscle results in sustained expression and systemic delivery of a therapeutic protein,” Proc. Natl. Acad. Sci. USA, 93:14082-87, 1996.
Klein et al., Exp. Neurol., 150:183-94, 1998.
Knoell, Ralston, Coulter and Wewers, “Alpha 1-antitrypsin and protease complexation is induced by lipopolysaccharide, interleukin-1β, and tumor necrosis factor-alpha in monocytes,” Am. J. Respir. Crit. Care Med., 157:246-55, 1998.
Kotin, Linden and Berns, “Characterization of a preferred site on human chromosome 19q for integration of adeno-associated virus DNA by non-homolgous recombination,” EMBO Journal, 11:5071-78, 1992.
Kotin, Siniscalco, Samulski, Zhu, Hunter, Laughlin, McLaughlin, Muzyczka, Rocchi and Berns, “Site-specific integration by adeno-associated virus,” Proc. Natl. Acad. Sci. USA, 87:2211-15, 1990.
Kroemer, Hirsch, Gonzalez-Garcia and Martinez, “Differential involvement of Th1 and Th2 cytokines in autoimmune diseases,” Autoimmunity, 24:25-33, 1996.
Li et al., J. Virol., 71:5236-43, 1997.
Li, Eastman, Schwartz and Draghia-Akli, Nat. Biotechnol., 17:241-45, 1999.
Liblau, Singer and McDevitt, “Th1 and Th2CD4+ T-cells in the pathogenesis of organ specific autoimmune diseases,” Immunology Today, 16:34-38, 1995.
Like and Rossini, “Streptozotocin-induced pancreatic insulitis: new model of diabetes mellitus,” Science, 193:415-17, 1976.
Like, Biron, Weringer, Byman, Sroczynski and Guberski, “Prevention of diabetes in BioBreeding/Worcester rats with monoclonal antibodies that recognize T lymphocytes or natural killer cells,” J. Exp. Med., 164:1145-59, 1986.
Linetsky, Bottino, Lehmann, Alejandro, Inverardi and Ricordi, “Improved human islet isolation using a new enzyme blend, liberase,” Diabetes, 46:1120-23, 1997.
Linetsky, Inverardi, Kenyon, Alejandro and Ricordi, “Endotoxin contamination of reagents used during isolation and purification of human pancreatic islets,” Transplant Proc., 30:345-46, 1998.
Lusby, Fife and Berns, “Nucleotide sequence of the inverted terminal repetition in adeno-associated virus DNA,” J. Virol., 34:402-09, 1980.
Macen, Upton, Nation and McFadden, “SERP1, a serine proteinase inhibitor encoded by myxoma virus, is a secreted glycoprotein that interferes with inflammation,” Virology, 195:348-63, 1993.
Massetti, Inverardi, Ranuncoli, Iaria, Lupo, Vizzardelli, Kenyon, Alejandro and Ricordi, “Current indications and limits of pancreatic islet transplantation in diabetic nephropathy,” J. Nephrol., 10:245-2521, 1997.
McAuthor and Raulet, “CD28-induced costimulation of T helper type 2 cells mediated by induction of responsiveness to interleukin 4,” J. Exp. Med., 178:1645, 1993.
Miller, Appel, O'Neil and Wicker, “Both the Lyt-2+ and L3T4+ T cell subsets are required for the transfer of diabetes in nonobese diabetic mice,” J. Immunol., 140:52-58, 1988.
Mitchell and Tjian, Science, 245:371-78, 1989.
Miyamoto, Akaike, Alam, Inoue, Hamamoto, Ikebe, Yoshitake, Okamoto and Maeda, “Novel functions of human α(1)-protease inhibitor after S-nitrosylation: inhibition of cysteine protease and antibacterial activity,” Biochem. Biophys. Res. Commun., 267:918-23, 2000.
Moritani, Yoshimoto, Tashiro, Hashimoto, Miyazaki, Li, Kudo, Iwahana, Hayashi, Sano et al., “Transgenic expression of IL-10 in pancreatic islet A cells accelerates autoimmune insulitis and diabetes in non-obese diabetic mice,” Int. Immunol., 6:1927-36, 1994.
Mueller, Krahl and Sarvetnick, “Pancreatic expression of interleukin-4 abrogates insulitis and autoimmune diabetes I nonobese diabetic (NOD) mice,” J. Exp. Med., 184:1093-99, 1996.
Murphy, Zhou, Giese, Williams, Escobedo and Dwarki, “Long-term correction of obesity and diabetes in genetically obese mice by a single intramuscular injection of recombinant adeno-associated virus encoding mouse leptin,” Proc. Natl. Acad. Sci. USA, 94:13921-26, 1997.
Muzyczka, “Use of adeno-associated virus as a general transduction vector for mammalian cells,” Curr. Top. Microbiol. Immunol., 158:97-129, 1992.
Nettelbeck, Jerome and Muller, Gene Ther., 5:1656-64, 1998.
Ni, Zhou, McCarty, Zolotukhin and Muzyczka, “In vitro replication of adeno-associated virus DNA,” J. Virol., 68:1128-38, 1994.
Nickerson, Steurer, Steiger, Zheng, Steele and Strom, “Cytokines and the Th1/Th2 paradigm in transplantation,” Curr. Opin. Immunol., 6:757-64, 1994.
Niemann, Baggott and Miller, “Binding of SPAAT, the 44-residue C-terminal peptide of alpha 1-antitrypsin, to proteins of the extracellular matrix,” J. Cell Biochem., 66:346-57, 1997.
Nitta, Tashiro, Tokui, Shimada, Takei, Tabayashi and Miyazaki, “Systemic delivery of interleukin 10 by intramuscular injection of expression plasmid DNA prevents autoimmune diabetes in nonobese diabetic mice,” Hum. Gene Ther., 9:1701-07, 1998.
Nussler, Carroll, Di Silvio, Rilo, Simmons, Starzl and Ricordi, “Hepatic nitric oxide generation as a putative mechanism for failure of intrahepatic islet cell grafts,” Transplant Proc., 24:2997, 1992.
O'Blenes, Zaidi, Cheah, McIntyre, Kaneda and Rabinovitch, “Gene transfer of the serine elastase inhibitor elafin protects against vein graft degeneration,” Circulation, 102:III289-95, 2000.
Oldstone, “Prevention of Type I diabetes in Nonobese Diabetic Mice by Virus Infection,” Science, 23:500, 1988.
Olsen et al., “Alpha-1-antitrypsin content in the serum, alveolar macrophages, and alveolar lavage fluid of smoking and nonsmoking normal subjects,” J. Clin. Invest., 55:427-430, 1975.
Parish, Chandler, Quartey-Papafio, Simpson and Cooke, “The effect of bone marrow and thymus chimerism between non-obese diabetic (NOD) and NOD-E transgenic mice, on the expression and prevention of diabetes,” Eur. J. Immunol., 23:2667, 1993.
Peltier and Hansen, “Immunoregulatory activity, biochemistry, and phylogeny of ovine uterine serpin,” Am. J. Reprod. Immunol., 45:266-72, 2001.
Penn, “Why do immunosuppressed patients develop cancer?,” Crit. Rev. Onogen., 1:27-52, 1989.
Pennline, Roque-Gaffney and Monahan, “Recombinant human IL-10 prevents the onset of diabetes in the nonobese diabetic mouse,” Clin. Immunol. Immunopathol., 71:169-75, 1994.
Perlino, Cortese and Ciliberto, “The human alpha 1-antitrypsin gene is transcribed from two different promoters in macrophages and hepatocytes,” Embo. J, 6:2767-71, 1987.
Perlmutter and Punsal, “Distinct and additive effects of elastase and endotoxin on expression of al proteinase inhibitor in mononuclear phagocytes,” J. Biol. Chem., 263:16499-503, 1988.
Perlmutter et al., “Expression of the alpha 1-proteinase inhibitor gene in human monocytes and macrophages,” Proc. Nat'l Acad. Sci. USA, 82:795-799, 1985.
Perlmutter et al., “Identification of a serpin-enzyme complex receptor on human hepatoma cells and human monocytes,” Proc. Nat'l Acad. Sci. USA, 87:3753-57, 1990.
Perlmutter, May and Sehgal, “Interferon beta 2/interleukin 6 modulates synthesis of alpha 1-antitrypsin in human mononuclear phagocytes and in human hepatoma cells,” J. Clin. Invest., 84:138-144, 1989.
Phillips, Parish, Drage and Cooke, “Cutting edge: interactions through the IL-10 receptor regulate autoimmune diabetes,” J. Immunol., 167:6087-91, 2001.
Pitluk and Ward, J. Virol., 65:6661-70, 1991.
Ponnazhagan, Erikson, Kearns, Zhou, Nahreini, Wang and Srivastava, “Lack of site-specific integration of the recombinant adeno-associated virus 2 genomes in human cells,” Hum. Gene Ther., 8:275-84, 1997.
Prasad, Yang, Bleich and Nadler, “Adeno-associated virus vector mediated gene transfer to pancreatic β cells,” Gene Ther., 7:1553-61, 2000.
Qing, Mah, Hansen, Zhou, Dwarki and Srivastava, “Human fibroblast growth factor receptor 1 is a co-receptor for infection by adeno-associated virus 2,” Nat. Med., 5:71-77, 1999.
Rabinovitch, “An update on cytokines in the pathogenesis of insulin-dependent diabetes mellitus,” Diabetes Metab. Rev., 14:129-51, 1998.
Rabinovitch, “Immunoregulatory and cytokine imbalances in the pathogenesis of IDDM: therapeutic intervention by immunostimulation?,” Diabetes, 44:613-621, 1994.
Rabinovitch, Suarez-Pinzon, Sorensen, Bleackley, Power and Rajotte, “Combined therapy with interleukin-4 and interleukin-10 inhibits autoimmune diabetes recurrence in syngeneic islet-transplanted nonobese diabetic mice. Analysis of cytokine mRNA expression in the graft,” Transplantation, 60:368-74, 1995.
Rabinowitz and Samulski, “Adeno-associated virus expression systems for gene transfer,” Curr. Opin. Biotechnol., 9:470-75, 1998.
Rapoport, Jaramillo, Zipris, Lazarus, Serreze, Leiter, Cyopick, Danska and Delovitch, “Interleukin 4 reverses T cell proliferative unresponsiveness and prevents the onset of diabetes in nonobese diabetic mice,” J. Exp. Med., 178:87-99, 1993.
Ray, Desmet and Gepts, “α-1-Antitrypsin immunoreactivity in islet cells of adult human pancreas,” Cell Tissue Res., 185:63-68, 1977.
Rendahl, Leff, Otten, Spratt, Bohl, Roey, Donahue, Cohen, Mandel, Danos and Smyder, “Regulation of gene expression in vivo following transduction by two separate rAAV vectors,” Nature Biotech. 16:757-62, 1998.
Ricordi, Lacy, Finke, Olack and Scharp, “Automated method for isolation of human pancreatic islets,” Diabetes, 37:413-20, 1988.
Robbins and Evans, “Prospects for treating autoimmune and inflammatory diseases by gene therapy,” Gene Therapy, 3:187-89, 1996.
Robertson, “Pancreatic islet cell transplantation: likely impact on current therapeutics for Type 1 diabetes mellitus,” Drugs, 61:2017-20, 2001.
Rosenberg, “Clinical islet cell transplantation. Are we there yet?,” Int. J. Pancreatol., 24:145-68, 1998.
Rossini, Like, Chick, Appel and Cahill. “Studies of streptozotocin-induced insulitis and diabetes,” Proc. Natl. Acad. Sci. USA, 74:2485-89, 1977.
Sallenave and Ryle, “Purification and characterization of elastase-specific inhibitor. Sequence homology with mucus proteinase inhibitor,” Biol. Chem. Hoppe-Seyler, 372:13-21, 1991.
Samulski, Chang and Shenk, “Helper-free stocks of recombinant adeno-associated viruses: normal integration does not require viral gene expression,” J. Virol., 63:3822-28, 1989.
Sandelain, Qin, Lauzon and Singh, “Prevention of type 1Type I diabetes in NOD mice by adjuvant immunotherapy,” Diabetes, 39:583, 1990.
Scharp, Lacy, Santiago, McCullough, Weide, Boyle, Falqui, Marchetti, Ricordi, Gingerich et al., “Results of our first nine intraportal islet allografts in Type 1Type I, insulin-dependent diabetic patients,” Transplantation, 51:76-85, 1991.
Schmidt-Wolf and Schmidt-Wolf, “Cytokines and gene therapy,” Immunology Today, 16:173-75, 1995.
Serreze, “Autoimmune diabetes results from genetic defects manifest by antigen presenting cells,” FASEB J, 7:1092-96, 1993.
Sharp, “The current status of alpha-1-antityrpsin, a protease inhibitor, in gastrointestinal disease,” Gastroenterology, 70:611-21, 1976.
She, Ellis, Wilson, Wasserfall, Marron, Reimsneider, Kent, Hafler, Neuberg, Muir, Strominger and Atkinson, “Heterophile antibodies segregate in families and are associated with protection from type 1 Type I diabetes,” Proc. Natl. Acad. Sci. USA, 96:8116-19, 1999.
Shehadeh, Clacinaro, Bradley, Bruchlim, Vardi and Lafferty, “Effect of adjuvant therapy on the development of diabetes in mouse and man,” The Lancet, 343:706, 1994.
Shelburne and Ryan, “The role of Th2 cytokines in mast cell homeostasis,” Immunol. Rev., 179:82-93, 2001.
Sibley and Sutherland, “Pancreas transplantation. An immunohistologic and histopathologic examination of 100 grafts,” Am. J. Pathol., 128:151-70, 1987.
Smith, Korbutt, Suarez-Pinzon, Kao, Rajotte and Elliott, “Interleukin-4 or interleukin-10 expressed from adenovirus-transduced syngeneic islet grafts fails to prevent β cell destruction in diabetic NOD mice,” Transplantation, 64:1040-49, 1997.
Snyder, Miao, Patijn, Spratt, Danos, Nagy, Gown, Winther, Meuse, Cohen, Thompson and Kay, “Persistent and therapeutic concentrations of human factor IX in mice after hepatic gene transfer of recombinant AAV vectors,” Nat. Genet., 16:270-76, 1997b.
Snyder, Spratt, Lagarde, Bohl, Kaspar, Sloan, Cohen and Danos, “Efficient and stable adeno-associated virus-mediated transduction in the skeletal muscle of adult immunocompetent mice,” Hum. Gene Ther., 8:1891-900, 1997a.
Socci, Falqui, Davalli, Ricordi, Braghi, Bertuzzi, Maffi, Secchi, Gavazzi, Freschi et al., “Fresh human islet transplantation to replace pancreatic endocrine function in Type 1Type I diabetic patients. Report of six cases,” Acta Diabetol., 28:151-57, 1991.
Song, Embury, Laipis, Berns, Crawford and Flotte, “Stable therapeutic serum levels of human alpha-1 antitrypsin (AAT) after portal vein injection of recombinant adeno-associated virus (rAAV) vectors,” Gene Ther., 8:1299-306, 2001b2001a.
Song, Laipis, Berns and Flotte, “Effect of DNA-dependent protein kinase on the molecular fate of the rAAV2 genome in skeletal muscle,” Proc. Natl. Acad. Sci. USA, 98:4084-88, 2001b.
Song, Morgan, Ellis, Poirier, Chestnut, Wang, Brantly, Muzyczka, Byrne, Atkinson and Flotte, “Sustained secretion of human α-1-antitrypsin from murine muscle transduced with adeno-associated virus vectors,” Proc. Natl. Acad. Sci. USA, 95:14384-88, 1998.
Srivastava, Lusby and Berns, “Nucleotide sequence and organization of the adeno-associated virus 2 genome,” J. Virol., 45:555-64, 1983.
Stein and Carrell, “What do dysfunctional serpins tell us about molecular mobility and disease?,” Nat. Struct. Biol., 2:96-113, 1995.
Stevens, Lokeh, Ansite, Field, Gores and Sutherland, “Role of nitric oxide in the pathogenesis of early pancreatic islet dysfunction during rat and human intraportal islet transplantation,” Transplant Proc., 26:692, 1994.
Stewart, A. F., Richard, III, C. W., Suzow, J., Stephan D., Weremowicz, S., Morton, C. C., Andra, C. N. (1996) Genomics 37(1):68-76.
Summerford and Samulski, “Membrane-associated heparan sulfate proteoglycan is a receptor for adeno-associated virus type 2 virions,” J. Virol., 72:1438-45, 1998.
Summerford, Bartlett and Samulski, “αᵥβ₅ integrin: a co-receptor for adeno-associated virus type 2 infection,” Nat. Med., 5:78-82, 1999.
Taylor-Robinson and Phillips, “Expression of IL-1 receptor discriminates Th2 from Th1 cloned CD4+ T cells specific for Plasmodium chabaudi,” Immunology, 81:216, 1994.
Tian, Olcott, Hanssen, Zekzer, Middleton and Kaufman, “Infectious Th1 and Th2 autoimmunity in diabetes-prone mice,” Immunol. Rev., 164:119-27, 1998.
Tremblay, Sallenave, Israel-Assayag, Cormier and Gauldie, “Elafin/elastase-specific inhibitor in bronchoalveolar lavage of normal subjects and farmer's lung,” Am. J. Respir. Crit. Care Med., 154:1092-98, 1996.
Trudeau, Dutz, Arany, Hill, Fieldus and Finegood, “Neonatal β-cell apoptosis: a trigger for autoimmune diabetes?,” Diabetes, 49:1-7, 2000.
Venkatesan, Davidson and Hutchinson, “Possible role for the glucose-fatty acid cycle in dexamethasone-induced insulin antagonism in rats,” Metabolism, 36:883-91, 1987.
Wang, Hao, Gill and Lafferty, “Autoimmune diabetes in NOD mouse is L3T4 T-lymphocyte dependent,” Diabetes, 36:535-38, 1987.
Warnock, Kneteman, Ryan, Seelis, Rabinovitch and Rajotte, “Normoglycaemia after transplantation of freshly isolated and cryopreserved pancreatic islets in Type 1Type I (insulin-dependent) diabetes mellitus,” Diabetologia, 34:55-58, 1991.
Waugh, Li-Hawkins, Yuksel, Cifra, Amabile, Hilfiker, Geske, Kuo, Thomas, Dake and Woo, “Therapeutic elastase inhibition by α-1-antitrypsin gene transfer limits neointima formation in normal rabbits,” J. Vasc. Interv. Radiol., 12:1203-09, 2001.
Wegmann and Eisenbarth, “It's insulin,” J. Autoimmun., 15:286-91, 2000.
Wei et al., J. Biol. Chem., 258:13506-512, 1993.
Weir, Bonner-Weir and Leahy, “Islet mass and function in diabetes and transplantation,” Diabetes, 39:401-05, 1990.
Wiedow, Schroder, Gregory, Young and Christophers, “Elafin: an elastase-specific inhibitor of human skin. Purification, characterization, and complete amino acid sequence,” J. Biol. Chem., 265:14791-95, 1990.
Wogensen, Huang and Sarvetnick, “Leukocyte extravasation into the pancreatic tissue in transgenic mice expressing interleukin 10 in the islets of Langerhans,” J. Exp. Med., 178:175-85, 1993.
Wogensen, Lee and Sarvetnick, “Production of interleukin 10 by islet cells accelerates immune-mediated destruction of β cells in nonobese diabetic mice,” J. Exp. Med., 179:1379-84, 1994.
Wong and Janeway, “The role of CD4 vs. CD8 T cells in IDDM,” J. Autoimmun., 13:290-95, 1999.
Xiao, Berta, Lu, Moscioni, Tazelaar and Wilson, “Adeno-associated virus as a vector for liver-directed gene therapy,” J. Virol., 72:10222-26, 1998.
Xiao, Li and Samulski, “Efficient long-term gene transfer into muscle tissue of immuno-competent mice by adeno-associated virus vector,” J. Virol., 70:8098-108, 1996.
Xu, Daly, Gao, Flotte, Song, Byrne, Sands and, Parker-Ponder, “CMV-β-actin promoter directs higher expression from an adeno-associated viral vector in the liver than the cytomegalovirus or elongation factor 1 α promoter and results in therapeutic levels of human factor X in mice,” Hum. Gene Ther., 12:563-73, 2001.
Yan, Zhang, Duan and Engelhardt, “From the cover: trans-splicing vectors expand the utility of adeno-associated virus for gene therapy,” Proc. Natl. Acad. Sci. USA, 97:6716-21, 2000.
Yoon, Jun and Santamaria, “Cellular and molecular mechanisms for the initiation and progression of β cell destruction resulting from the collaboration between macrophages and T cells,” Autoimmunity, 27:109-22, 1998.
Yu, Robles, Abiru, Kaur, Rewers, Kelemen and Eisenbarth, “Early expression of antiinsulin autoantibodies of humans and the NOD mouse: evidence for early determination of subsequent diabetes,” Proc. Natl. Acad. Sci. USA, 97:1701-06, 2000.
Zaidi, Hui, Cheah, You, Husain and Rabinovitch, “Targeted overexpression of elafin protects mice against cardiac dysfunction and mortality following viral myocarditis,” J. Clin. Invest., 103:1211-19, 1999.
Zhang et al., “Adeno-associated virus transduction of islets with interleukin-4 results in impaired metabolic function in syngeneic marginal islet mass transplantation,” Transplantation, 74: in press, 2002b.
Zhang et al., “Genetic predisposition to autoimmunity specifically imparts responsiveness to transgenes delivered by recombinant adeno-associated virus,” Mol. Ther., 5:S430 (Abstr. 1317), 2002a.
Ziady et al., “Chain length of the polylysine in receptor-targeted gene transfer complexes affects duration of reporter gene expression both in vitro and in vivo,” J. Biol. Chem., 274:4908-16, 1999.
Zolotukhin, Potter, Hauswirth, Guy and Muzyczka, J. Virol., 70:4646-54, 1996.
Zolotukhin, Zolotukhin, Byrne, Mason, Zolotukhin, Potter, Chesnut, Summerford, Samulski and Muzyczka, “Recombinant adeno-associated virus purification using novel methods improves infectious titer and yield,” Gene Ther., 6:973-985, 1999.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An adeno-associated viral vector comprising at least a first polynucleotide that comprises a mammalian β-actin promoter and a woodchuck hepatitis virus post-transcriptional regulatory sequence, each operably linked to an isolated nucleic acid segment that encodes a biologically-active mammalian interleukin polypeptide selected from the group consisting of IL-10 and IL-10(I87A), wherein said promoter expresses said nucleic acid segment in a mammalian pancreatic islet cell that comprises said vector to produce said encoded mammalian interleukin polypeptide.

2-42. (canceled)

43. A method for preventing, treating or ameliorating the symptoms of an interleukin polypeptide deficiency in a mammal, said method comprising administering to said mammal a composition comprising an adeno-associated viral vector that comprises at least a first polynucleotide that comprises a promoter operably positioned upstream of an isolated nucleic acid segment encoding a biologically-active mammalian interleukin polypeptide selected from the group consisting of IL-10 and IL-10(I87A), wherein said promoter expresses said nucleic acid segment in a mammalian pancreatic islet cell that comprises said vector to produce said encoded mammalian interleukin polypeptide, in an amount and for a time sufficient to treat or ameliorate the symptoms of said deficiency in said mammal.

44. The method of claim 43, wherein said mammal is a human.

45. The method of claim 44, wherein said mammal has, is diagnosed with, or is at risk for developing Type I diabetes.

46. The method of claim 43, wherein said virion or said plurality of viral particles is administered to said mammal intramuscularly, intravenously, subcutaneously, intrathecally, intraperitoneally, or by direct injection into an organ or a tissue.

47. The method of claim 46, wherein said organ or tissue is selected from the group consisting of pancreas, liver, heart, lung, brain, kidney, joint, and muscle.

48. A method for treating diabetes in a mammal suspected of having, or at risk for developing diabetes, said method comprising providing to said mammal a composition comprising an adeno-associated viral vector that comprises at least a first polynucleotide that comprises a promoter operably positioned upstream of an isolated nucleic acid segment encoding a biologically-active mammalian interleukin polypeptide selected from the group consisting of IL-10 and IL-10(I87A), wherein said promoter expresses said nucleic acid segment in a mammalian pancreatic islet cell that comprises said vector to produce said encoded mammalian interleukin polypeptide, in an amount and for a time sufficient to treat said diabetes in said mammal.

49. The method of claim 48, wherein said mammal is human.

50. The method of claim 49, wherein said mammal is human with a familial history of diabetes.

51. A method for preventing Type I diabetes in a human suspected of having, or at risk for developing Type I diabetes, said method comprising prophylactically administering to said human a composition comprising an adeno-associated viral vector that comprises at least a first polynucleotide that comprises a promoter operably positioned upstream of an isolated nucleic acid segment encoding a biologically-active mammalian interleukin polypeptide selected from the group consisting of IL-10 and IL-10(I87A), wherein said promoter expresses said nucleic acid segment in a mammalian pancreatic islet cell that comprises said vector to produce said encoded mammalian interleukin polypeptide, in an amount and for a time sufficient to prevent said Type I diabetes from developing in said human.

52. A method for reducing the rate of disease progression of Type I diabetes in a human diagnosed with Type I diabetes, said method comprising administering to said human a composition comprising an adeno-associated viral vector that comprises at least a first polynucleotide that comprises a promoter operably positioned upstream of an isolated nucleic acid segment encoding a biologically-active mammalian cytokine polypeptide, wherein said promoter expresses said nucleic acid segment in a mammalian pancreatic islet cell that comprises said vector to produce said encoded mammalian cytokine polypeptide, in an amount and for a time sufficient to reduce the rate of disease progression of said Type I diabetes in said human.

53. The method of claim 52, wherein said cytokine is an interleukin.

54. The method of claim 53, wherein said interleukin is an IL-4 or an IL-10 polypeptide.

55. The method of claim 54, wherein said IL-10 polypeptide comprises an isoleucine to alanine mutation at amino acid 87 (I87A).

56. A method for treating Type I diabetes in a mammal, said method comprising providing to said mammal a composition comprising an adeno-associated viral vector that comprises at least a first polynucleotide that comprises a mammalian β-actin promoter operably linked to an isolated nucleic acid segment encoding a biologically-active mammalian interleukin polypeptide selected from the group consisting of IL-10 and IL-10(I87A), wherein said mammalian β-actin promoter expresses said nucleic acid segment in a mammalian pancreatic islet cell that comprises said vector to produce said encoded mammalian interleukin polypeptide, in an amount and for a time sufficient to treat said Type I diabetes in said mammal.

57. The method of claim 56, wherein said interleukin polypeptide comprises the amino acid sequence of SEQ ID NO:1 or SEQ ID NO:3.

58. The method of claim 56, wherein said vector further comprises a woodchuck hepatitis virus post-transcriptional regulatory sequence operably linked to said nucleic acid segment.


SEQUENCE LISTING (54 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - protein, 153 aa, Homo sapiens [sequence omitted]

SEQ ID NO: 4 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 5 - protein, 107 aa, Homo sapiens
MTQPGVLRSAAARKPGYCPEFDLDCPFTLLPMRWRDKSCRGSRSVATTTVGISVWSPGGLWIEVRSYPLCKSFEERSYPFCESFKDQQTSEHPACREEPPSPGPPLC

SEQ ID NO: 6 - protein, 89 aa, Maccus mullatus
VVVFLIAGMLVVEAAVTGVPVKGQDTVKGRVPFNGQDPVKGQVSVKGQDRVKGRGPVKGPVSTKPGSCPNILIRCAMLNPPNRCLKDTD

SEQ ID NO: 7 - protein, 143 aa, Sus scrofa
MRSRSFLVLVVVFLICGTLVVQAAGRIRRPKGKGTKKTLALVKGQGPVRGKDQVKGQGPVKGQDLGKSQDPVKAQLPDKGQDPVKAQPAIKRLILLTKPGSCPRILIRCLMVNPPNRCLSDAQCPGVKKCCEGFCGKDCMDPK

SEQ ID NO: 8 - protein, 167 aa, Sus scrofa
MRSRSFLVLVVVFLICGTLVAQAAGRIRRPKGKGTKKILALVKGQGPVRGKDQVKGQGPVKGQDLGKSQDPVKAQLPDKGQDLGKGEDSVKGQDPFKAQLPDKLQDPVKAQPAIKRLILLTKPGSCPRILIRCLMVNPPNRCLSDAQCPGLKKCCEGFCGKACMDPK

SEQ ID NO: 9 - protein, 96 aa, Bos taurus
SPKGQGNVVFNGKGPVNGQSPDKGQDPVKGQDPVKGQDVVVAQDRAGLPFKRGLCPRVRIHCNLWNPPNQCWRDAHCPGAKKCCEGFCGKTCMNPR

SEQ ID NO: 10 - protein, 131 aa, Rattus norvegicus
MKSCGLFPLMVLLALGVLAPWSVEGGKNDAIKIGACPARKPAQCLKLEKPECGTDWECPGKQRCCQDTCGFKCLNPVPIRGPVKKKPGRCVKFQGKCLMLNPPNKCQNDGQCDGKYKCCEGMCGKVCLPPV

SEQ ID NO: 11 - protein, 131 aa, Mus musculus
MKSCGLLPFTVLLALGILAPWTVEGGKNDAIKIGACPAKKPAQCLKLEKPQCRTDWECPGKQRCCQDACGSKCVNPVPIRKPVWRKPGRCVKTQARCMMLNPPNVCQRDGQCDGKYKCCEGICGKVCLPPM

SEQ ID NO: 12 - protein, 397 aa, Rattus norvegicus
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITRSENLHVSHILQKAKIEVSEDGTKASAATTAILIARSSPPWFIVDRPFLFFIRHNPTGAVLFMGQINKP

SEQ ID NO: 13 - protein, 374 aa, Homo sapiens
MNTLSEGNGTFAIHLLKMLCQSNPSKNVCYSPASISSALAMVLLGAKGQTAVQISQALGLNKEEGIHQGFQLLLRKLNKPDRKYSLRVANRLFADKTCEVLQTFKESSLHFYDSEMEQLSFAEEAEVSRQHINTWVSKQTEGKIPELLSGGSVDSETRLVLINALYFKGKWHQPFNKEYTMDMPFKINKDEKRPVQMMCREDTYNLAYVKEVQAQVLVMPYEGMELSLVVLLPDEGVDLSKVENNLTFEKLTAWMEADFMKSTDVEVFLPKFKLQEDYDMESLFQRLGVVDVFQEDKADLSGMSPERNLCVSKFVHQSVVEINEEGTEAAAASAIIEFCCASSVPTFCADHPFLFFIRHNKANSILFCGRFSSP

SEQ ID NO: 14 - protein, 410 aa, Cyprinus carpio
MAWAAPHEGHDHDGHPADHYHHLHHGKDEAHPSHSGEDACHLLSPHNADFAFSLYKKLALHPDAQGKNIFFSPVGISMALSMLAVGAKGSTLSQIYSSLGYSGLKAQQVNEGYEHLIHMLGHSQDTMQLEAGAGVAIREGFKVVDQFLKDVQHYYNSEAFSVDFSKPEIAAEEINQFIAKKTNDKITDMVKDLDSDMVMMLINYMYFRGKWDKPFEAQLTHKAEFKVDKDTTVQVDMMKRTGRYDIYQDPVNQTTVMMVPYKGNTSMMIVLPDEGKMKDVEESICRHHLKNWHDKLFRSSVDLFMPKFSISATSKLNDILTEMGVTDAFSDTADFSGMTEELKVKVSQVVHKAVLSVDEKGTEAAAATTIEIMPMSLPGTVMLNRPFLVLIVEDTTKSILFMGKITNPTV

SEQ ID NO: 15 - protein, 378 aa, Sus scrofa
  MISC_FEATURE (337)..(337): X = ANY AMINO ACID
MEQLSAANTRFALDLFRALNESNPAGNIFISPFSISSALAMILLGTRGNTEAQMSKALHFDTVKDIHSRFQSLNADINKCGASYILKLANRLFGEKTYHFLPEFLASTQKTYGAELASVDFLRASEEARKAINEWVKEQTEGKIPELLASGVVDSATKLVLVNAIYFKGSWQEKFMTEATKDAPFRLNKKDSKTVKMMYQKKKFPFGYIKELKCRVLELPYQGKDLSMVILLPDSIEDESTGLRKIEQHLTLEKLREWTKPDNLELLEVNVHLPRFRLEESYDLNAPLARLGVQDLFGSRADLTGMSEARDLFISKVVHKSFVEVNEEGTEAAAATXGIAVFAMLMPEEDFIADHPFIFFIRHNPSSNILFLGRLSSP

SEQ ID NO: 16 - protein, 54 aa, Equus caballus
EDLQGDAVPERHATKDDNEHPQEPAEHKKAPNEAIRTLLHTNVEFNRPFVLIIY

SEQ ID NO: 17 - protein, 49 aa, Equus caballus
EDLQGDAVPERHATKDDNEHPQEPAEHKKAPNERPATLLLDNVEFNRPF

SEQ ID NO: 18 - protein, 54 aa, Equus caballus
EDLQGDAVPERHATKDDNEHPQEPAEHKKAPNEMIPMSLPPELEFNRPFILIIY

SEQ ID NO: 19 - protein, 390 aa, Homo sapiens
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

SEQ ID NO: 20 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 21 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 22 - protein, 410 aa, Gallus gallus
MYFLGLLSLLVLPSKAFKTNFPDETIAELSVNVYNQLRAAREDENILFCPLSIAIAMGMIELGAHGTTLKEIRHSLGFDSLKNGEEFTFLKDLSDMATTEESHYVLNMANSLYVQNGFHVSEKFLQLVKKYFKAEVENIDFSQSAAVATHINKWVENHTNNMIKDFVSSRDFSALTHLVLINAIYFKGNWKSQFRPENTRTFSFTKDDETEVQIPMMYQQGEFYYGEFSDGSNEAGGIYQVLEIPYEGDEISMMIVLSRQEVPLVTLEPLVKASLINEWANSVKKQKVEVYLPRFTVEQEIDLKDVLKGLGITEVFSRSADLTAMSDNKELYLAKAFHKAFLEVNEEGSEAAAASGMIAISRMAVLYPQVIVDHPFFFLVRNRRTGTVLFMGRVMHPEAMNTSGHDFEEL

SEQ ID NO: 23 - protein, 648 aa, Mus musculus
MSLKWSACWVALGQLLCSCALALKGGMLFPKESPSRELKALDGLWHFRADLSNNRLQGFEQQWYRQPLRESGPVLDMPVPSSFNDITQEAALRDFIGWVWYEREAILPRRWTQDTDMRVVLRINSAHYYAVVWVNGIHVVEHEGGHLPFEADISKLVQSGPLTTCRITIAINNTLTPHTLPPGTIVYKTDTSMYPKGYFVQDTSFDFFNYAGLHRSVVLYTTPTTYIDDITVITNVEQDIGLVTYWISVQGSEHFQLEVQLLDEDGKVVAHGTGNQGQLQVPSANLWWPYLMHEHPAYMYSLEVKVTTTESVTDYYTLPVGIRTVAVTKSKFLINGKPFYFQGVNKHEDSDIRGKGFDWPLLVKDFNLLRWLGANSFRTSHYPYSEEVLQLCDRYGIVVIDECPGVGIVLPQSFGNESLRHHLEVMEELVRRDKNHPAVVMWSVANEPSSALKPAAYYFKTLITHTKALDLTRPVTFVSNAKYDADLGAPYVDVICVNSYFSWYHDYGHLEVIQPQLNSQFENWYKTHQKPIIQSEYGADAIPGIHEDPPRMFSEEYQKAVLENYHSVLDQKRKEYVVGELIWNFADFMTNQSPLRVIGNKKGIFTRQRQPKTSAFILRERYWRIANETGGHGSGPRTQCFGSRPFTF

SEQ ID NO: 24 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 25 - protein, 413 aa, Oryctolagus cuniculus
MPPSVSRALLLLAGLGCLLPGFLADEAQETAVSSHEQDHPACHRIAPSLAEFALSLYREVAHESNTTNIFFSPVSIALAFAMLSLGAKGDTHTQVLEGLKFNLTETAEAQIHDGFRHLLHTVNRPDSELQLAAGNALVVHENLKLQHKFLEDAKNLYQSEAFLVDFRDPEQAKTKINSHVEKGTRGKIVDLVQELDARTLLALVNYVFFKGKWEKPFEPENTKEEDFHVDATTTVRVPMMSRLGMYVMFHCSTLASTVLRMDYKGNATALFLLPDEGKLQHLEDTLTTELIAKFLAKSSLRSVTVRFPKLSISGTYDLKPLLGKLGITQVFSNNADLSGITEQEPLKVSQALHKAVLTIDERGTEAAGASFVELIPESVPDSITLDRPFLFVIYSHEIKSPLFVGKVVDPTQH

SEQ ID NO: 26 - protein, 397 aa, Ratttus norvegicus
MNWHFPFFILTTVTLSSVYSQLNSLSLEELGSDTGIQVFNQIIKSQPHENVVISPHGIASILGMLQLGADGRTKKQLSTVMRYNVNGVGKVLKKINKAIVSKKNKDIVTVANAVFVRNGFKVEVPFAARNKEVFQCEVQSVNFQDPASACDAINFWVKNETRGMIDNLLSPNLIDSALTKLVLVNAVYFKGLWKSRFQPENTKKRTFVAGDGKSYQVPMLAQLSVFRSGSTKTPNGLWYNFIELPYHGESISMLIALPTESSTPLSAIIPHISTKTINSWMNTMVPKRMQLVLPKFTALAQTDLKEPLKALGITEMFEPSKANFAKITRSESLHVSHILQKAKIEVSEDGTKAAVVTTAILIARSSPPWFIVDRPFLFCIRHNPTGAILFLGQVNKP

SEQ ID NO: 27 - protein, 417 aa, Sus scrofa
MSHGKMPLVLSLVLILCGLFNSISCEKQQTSPKTITPVSFKRIAALSQKMEANYKAFAQELFKTLLIEDPRKNMIFSPVSISISLATLSLGLRSATRTNAIDVLDVALKNLAVMLMAQAPTALLEIVHELVNRTAKHQDILIDRTEMNQMFLKEIDRYIKMDIQMIDFKDKEKTKKAINQFVADKIDKKAKNLITHLDPQTLLCLVNYIFFKGILERAFQTNLTKKEDFFVNEKTIVQVDMMRKTERMIYSRSEELLATMVKIPCKENASIILVLPDTGKFNFALKEMAAKRARLQKTNDFRLVHLVVPKIKDNLQDRFKHLLPKIGINDIFTTKAVTWNTTGTSTILEAVHHAVIEVKEDGLTKNAAKDKDFWKVPVDKKEVPVVVKFDRPFFLFVEDEITRRDLFVAKVFNPKTE

SEQ ID NO: 28 - protein, 402 aa, Homo sapiens [sequence omitted]

SEQ ID NO: 29 - protein, 379 aa, Equus caballus
MEQLSTANTHFAVDLFRALNESDPTGNIFISPLSISSALAMIFLGTRGNTAAQVSKALYFDTVEDIHSRFQSLNADINKPGAPYILKLANRLYGEKTYNFLADFLASTQKMYGAELASVDFQQAPEDARKEINEWVKGQTEGKIPELLVKGMVDNMTKLVLVNAIYFKGNWQEKFMKEATRDAPFRLNKKDTKTVKMMYQKKKFPYNYIEDLKCRVLELPYQGKELSMIILLPDDIEDESTGLEKIEKQLTLEKLREWTKPENLYLAEVNVHLPRFKLEESYDLTSHLARLGVQDLFNRGKADLSGMSGARDLFVSKIIHKSFVDLNEEGTEAAAATAGTIMLAMLMPEENFNADHPFIFFIRHNPSANILFLGRFSSP

SEQ ID NO: 30 - protein, 417 aa, Homo sapiens
MRSLLLGTLCLLAVALAAEVKKPVEAAAPGTAEKLSSKATTLAEPSTGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFPDKRSALQSINEWAAQTTDGKLPEVTKDVERTDGALLVNAMFFKPHWDEKFHHKMVDNRGFMVTRSYTVGVTMMHRTGLYNYYDDEKEKLQLVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRLKGDKMRDEL

SEQ ID NO: 31 - protein, 418 aa, Homo sapiens [sequence omitted]

SEQ ID NO: 32 - protein, 476 aa, Ovis aries
MAPAGLSLGATILCLLAWAGLAAGDRVYIHPFHLLVHSKSNCDQLEKPSVETPADPTLTPVPIQTKSSPVDEEALWEQLVRATEKLEAEDRLRASEVGLLLNFMGFHVYKTLSETWSVASGLVFSPVALFSTLTSFYTGALDPTASRLQAFLGVPGEGQGCTSRLDGRKVLSSLQTIQGLLVAPGGASSQARLLLSTVVGLFTAPGLHLKQPFVQGLSSFAPITLPRSLDLSTDPNLAAEKINRFMHSATGWNMGRPLAAASPDSTLLFNAYVHFQGKMKGFSLLPGLTEFWVDNTTSVPVPMLSGSGTFHYWSDNQNHLSMTRVPLSANGYLLLIQPHHTLDLRKVEALIFQHNFLTRMKNLSPRAIHLTVPQLTLKASYDLQDLLAQAKLPTLLGAEANLGKISDANLRVGKVLNSVLFELKADGEQAPESVPQPAGPEALEVTLNSPFLLAVLERSSGALHFLGRVSRPLSAE

SEQ ID NO: 33 - protein, 397 aa, Mus musculus
MNWHFPFFILTTVTLYSVHSQFNSLSLEELGSNTGIQVFNQIIKSRPHENVVVSPHGIASILGMLQLGADGKTKKQLSTVMRYNVNGVGKVLKKINKAIVSKKNKDIVTVANAVFLRNGFKMEVPFAVRNKDVFQCEVQNVNFQDPASASESINFWVKNETRGMIDNLLSPNLIDGALTRLVLVNAVYFKGLWKSRFQPESTKKRTFVAGDGKSYQVPMLAQLSVFRSGSTRTPNGLWYNFIELPYHGESISMLIALPTESSTPLSAIIPHITTKTIDSWMNTMVPKRMQLVLPKFTAVAQTDLKEPLKALGITEMFEPSKANFTKITRSESLHVSHILQKAKIEVSEDGTKASAATTAILIARSSPPWFIVDRPFLFSIRHNPTGAILFLGQVNKP

SEQ ID NO: 34 - protein, 397 aa, Homo sapiens
MDSLATSINQFALELSKKLAESAQGKNIFFSSWSISTSLTIVYLGAKGTTAAQMAQVLQFNRDQGVKCDPESEKKRKMEFNLSNSEEIHSDFQTLISEILKPNDDYLLKTANAIYGEKTYAFHNKYLEDMKTYFGAEPQPVNFVEASDQIRKDINSWVERQTEGKIQNLLPDDSVDSTTRMILVNALYFKGIWEHQFLVQNTTEKPFRINETTSKPVQMMFMKKKLHIFHIEKPKAVGLQLYYKSRDLSLLILLPEDINGLEQLEKAITYEKLNEWTSADMMELYEVQLHLPKFKLEDSYDLKSTLSSMGMSDAFSQSKADFSGMSSARNLFLSNVFHKAFVEINEQGTEAAAGSGSEIDIRIRVPSIEFNANHPFLFFIRHNKTNTILFYGRLCSP

SEQ ID NO: 35 - protein, 376 aa, Homo sapiens
MDVLAEANGTFALNLLKTLGKDNSKNVFFSPMSMSCALAMVYMGAKGNTAAQMAQILSFNKSGGGGDIHQGFQSLLTEVNKTGTQYLLRVANRLFGEKSCDFLSSFRDSCQKFYQAEMEELDFISAVEKSRKHINTWVAEKTEGKIAELLSPGSVDPLTRLVLVNAVYFRGNWDGQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHRKTNGILFCGRFSSP

SEQ ID NO: 36 - protein, 406 aa, Homo sapiens
MQLFLLLCLVLLSPQGASLHRHHPREMKKRVEDLHVGATVAPSSRRDFTFDLYRALASAAPSQNIFFSPVSISMSLAMLSLGAGSSTKMQILEGLGLNLQKSSEKELHRGFQQLLQELNQPRDGFQLSLGNALFTDLVVDLQDTFVSAMKTLYLADTFPTNFRDSAGAMKQINDYVAKQTKGKIVDLLKNLDSNAVVIMVNYIFFKAKWETSFNHKGTQEQDFYVTSETVVRVPMMSREDQYHYLLDRNLSCRVVGVPYQGNATALFILPSEGKMQQVENGLSEKTLRKWLKMFKKRQLELYLPKFSIEGSYQLEKVLPSLGISNVFTSHADLSGISNHSNIQVSEMVHKAVVEVDESGTRAAAATGTIFTFRSARLNSQRLVFNRPFLMFIVDNNILFLGKVNRP

SEQ ID NO: 37 - protein, 491 aa, Homo sapiens
MALLWGLLVLSWSCLQGPCSVFSPVSAMEPLGRQLTSGPNQEQVSPLTLLKLGNQEPGGQTALKSPPGVCSRDPTPEQTHRLARAMMAFTADLFSLVAQTSTCPNLILSPLSVALALSHLALGAQNHTLQRLQQVLHAGSGPCLPHLLSRLCQDLGPGAFRLAARMYLQKGFPIKEDFLEQSEQLFGAKPVSLTGKQEDDLANINQWVKEATEGKIQEFLSGLPEDTVLLLLNAIHFQGFWRNKFDPSLTQRDSFHLDEQFTVPVEMMQARTYPLRWFLLEQPEIQVAHFPFKNNMSFVVLVPTHFEWNVSQVLANLSWDTLHPPLVWERPTKVRLPKLYLKHQMDLVATLSQLGLQELFQAPDLRGISEQSLVVSGVQHQSTLELSEVGVEAAAATSIAMSRMSLSSFSVNRPFLFFIFEDTTGLPLFVGSVRNPNPSAPRELKEQQDSPGNKDFLQSLKGFPRGDKLFGPDLKLVPPMEEDYPQFGSPK

SEQ ID NO: 38 - protein, 375 aa, Homo sapiens
MDALQLANSAFAVDLFKQLCEKEPLGNVLFSPICLSTSLSLAQVGAKGDTANEIGQVLHFENVKDIPFGFQTVTSDVNKLSSFYSLKLIKRLYVDKSLNLSTEFISSTKRPYAKELETVDFKDKLEETKGQINNSIKDLTDGHFENILADNSVNDQTKILVVNAAYFVGKWMKKFPESETKECPFRLNKTDTKPVQMMNMEATFCMGNIDSINCKIIELPFQNKHLSMFILLPKDVEDESTGLEKIEKQLNSESLSQWTNPSTMANAKVKLSIPKFKVEKMIDPKACLENLGLKHIFSEDTSDFSGMSETKGVALSNVIHKVCLEITEDGGDSIEVPGARILQHKDELNADHPFIYIIRHNKTRNIIFFGKFCSP

SEQ ID NO: 39 - protein, 416 aa, Rattus norvegicus
MAFIAALGLLMAGICPAVLCDGILGRDTLPHEDQGKGRQLHSLTLASINTDFTLSLYKKLALRNPDKNVVFSPLSISAALAILSLGAKDSTMEEILEGLKFNLTEITEEEIHQGFGHLLQRLSQPEDQAEINTGSALFIDKEQPILSEFQEKTRALYQAEAFVADFKQCNEAKKFINDYVSNQTQGKIAELFSELDERTSMVLVNYLLFKGKWKVPFNPNDTFESEFYLDEKRSVKVPMMKIKDLTTPYIRDEELSCSVLELKYTGNASALFILPDQGKMQQVESSLQPETLKKWKDSLRPRIISELRMPKFSISTDYNLEEVLPELGIRKIFSQQADLSRITGTKNLHVSQVVHKAVLDVDETGTEGAAATAVTAALKSLPQTIPLLNFNRPFMLVITDNNGQSVFFMGKVTNPM

SEQ ID NO: 40 - protein, 18 aa, Equus caballus
LAMLMPEENFNADHPFIF

SEQ ID NO: 41 - protein, 416 aa, Homo sapiens
MQALVLLLWTGALLGFGRCQNAGQEAGSLTPESTGAPVEEEDPFFKVPVNKLAAAVSNFGYDLYRVRSGESPTANVLLSPLSVATALSALSLGAEQRTESNIHRALYYDLISNPDIHGTYKDLLASVTAPQKNLKSASRIIFERKLRIKASFIPPLEKSYGTRPRILTGNSRVDLQEINNWVQAQMKGKVARSTREMPSEISIFLLGVAYFKGQWVTKFDSRKTSLEDFYLDEERTVKVPMMSDPQAVLRYGLDSDLNCKIAQLPLTGSTSIIFFLPQKVTQNLTLIEESLTSEFIHDIDRELKTVQAVLTIPKLKLSYEGELTKSVQELKLQSLFDAPDFSKITGKPIKLTQVEHRVGFEWNEDGAGTNSSPGVQPARLTFPLDYHLNQPFIFVLRDTDTGALLFIGKILDPRGT

SEQ ID NO: 42 - protein, 418 aa, Homo sapiens [sequence omitted]

SEQ ID NO: 43 - protein, 415 aa, Homo sapiens; identical to SEQ ID NO: 24

SEQ ID NO: 44 - protein, 415 aa, Homo sapiens
MSPFLYLVLLVLGLHATIHCASPEGKVTACHSSQPNATLYKMSSINADFAFNLYRRFTVETPDKNIFFSPVSISAALVMLSFGACCSTQTEIVETLGFNLTDTPMVEIQHGFQHLICSLNFPKKELELQIGNALFIGKHLKPLAKFLNDVKTLYETEVFSTDFSNISAAKQEINSHVEMQTKGKVVGLIQDLKPNTIMVLVNYIHFKAQWANPFDPSKTEDSSSFLIDKTTTVQVPMMHQMEQYYHLVDMELNCTVLQMDYSKNALALFVLPKEGQMESVEAAMSSKTLKKWNRLLQKGWVDLFVPKFSISATYDLGATLLKMGIQHAYSENADFSGLTEDNGLKLSNAAHKAVLHIGEKGTEAAAVPEVELSDQPENTFLHPIIQIDRSFMLLILERSTRSILFLGKVVNPTEA

SEQ ID NO: 45 - protein, 480 aa, Homo sapiens
MQHRPHLLLISLTIMSVCGGSNGLTDQLNNKNLTMPLLPIEFHKENTVTNDWIPEGEEDDDYLDLEKLLSEDDDYIDIIDAVSPTDSEASAGNILQLFQGKSRIQRLNILNAKFAFSLYRALKDQANAFDNIFIAPVGISTAMGMISLGLKGETHEQVHSVLHFRDFVNASSKYEILTIHNLFRKLTHRLFRRNFGYTLRSVNDLYVQKQFPIREDFKAKVREYYFAEAQAADFSDPAFISKANNHILKVTKGLIKEALENVDPATQMMILNCIYFKGTWVNKFPVEMTHNHNFRLNEREVVKVSMMQTKGNFLAANDQELACDVLQLEYVGGISMLIVVPHKLSGMKTLEAQLTPQVVERWQKSMTNRTREVLLPKFKLEKNYNLVEALKSMGVTELFDKNGNMSGISDQGITMDLFKHQGTITVNEEGTQAAAVTTVGFMPLSTQVRFTVDRPFLFLVYEHRTSCLLFMGKVANPVRS

SEQ ID NO: 46 - protein, 499 aa, Homo sapiens
MKHSLNALLIFLIITSAWGGSKGPLDQLEKGGETAQSADPQWEQLNNKNLSMPLLPADFHKENTVTNDWIPEGEEDDDYLDLEKIFSEDDDYIDIVDSLSVSPTDSDVSAGNILQLFHGKSRIQRLNILNAKFAFNLYRVLKDQVNTFDNIFIAPVGISTAMGMISLGLKGETHEQVHSILHFKDFVNASSKYEITTIHNLFRKLTHRLFRRNFGYTLRSVNDLYIQKQFPILLDFKTKVREYYFAEAQIADFSDPAFISKTNNHIMKLTKGLIKDALENIDPATQMMILNCIYFKGSWVNKFPVEMTHNHNFRLNEREVVKVSMMQTKGNFLAANDQELDCDILQLEYVGGISMLIVVPHKMSGMKTLEAQLTPRVVERWQKSMTNRTREVLLPKFKLEKNYNLVESLKLMGIRMLFDKNGNMAGISDQRIAIDLFKHQGTITVNEEGTQATTVTTVGFMPLSTQVRFTVDRPFLFLIYEHRTSCLLFMGRVANPSRS

SEQ ID NO: 47 - protein, 464 aa, Homo sapiens; identical to SEQ ID NO: 21

SEQ ID NO: 48 - protein, 362 aa, Homo sapiens [sequence omitted]

SEQ ID NO: 49 - protein, 410 aa, Homo sapiens; identical to SEQ ID NO: 14

SEQ ID NO: 50 - protein, 372 aa, Cyprinus carpio
MPATCLLHTMLTLPSPSTRNLRSIQMPRARTFSSPSRYRNGFEHAGCRCQGSTLSQIYSSLGYSGLQASQVNEGYEHLIHMLGHSREAMQLEAGAGVAIREGFKVVDQFLKDVQHYYNSEAFSVDFSKPEIAAEEINQFIAKKTNDKITNMVKDLDSDTVMMLINYMYFRGKWDKPFDAQLTHKADFKVDEDTTVQVDMMKRTGRYDIYQDPVNQTTVMMVPYKGNTSMMIIFPDDGKMKELEESISRHHLKNWHDKLFRSSVDLFMPKFSITATSKLKGILEDMGVTDAFGDTADLSGLTEEVKVKVSQVVHKAVLSVDEKGTEAAAATTIEIMPMSLPDTVILNRPFLVLIVEDTTKSILFMGKITNPTE

SEQ ID NO: 51 - DNA, 20 bp, Artificial Sequence
  Synthetic Oligonucleotide
cagtctcgaacttaagctgc

SEQ ID NO: 52 - DNA, 20 bp, Artificial Sequence
  Synthetic Oligonucleotide
ggacttggactcattcatgg

SEQ ID NO: 53 - DNA, 20 bp, Artificial Sequence
  Synthetic Oligonucleotide
cagaagttggtcgtgaggca

SEQ ID NO: 54 - DNA, 20 bp, Artificial Sequence
  Synthetic Oligonucleotide
gcagctccatgagaacacta